(19) United States
(12) Patent Application Publication
(10) Pub. No.: US 2024/0149064 A1
(43) Pub. Date: May 9, 2024

(54) SYSTEMS AND METHODS FOR TREATING BLADDER AND/OR BOWEL DYSFUNCTION

(71) Applicant: INSPIRE MEDICAL SYSTEMS, INC., Golden Valley, MN (US)

(72) Inventors: John Rondoni, Plymouth, MN (US); Stephen Lorne Bolea, Excelsior, MN (US); Christopher Poletto, North Oaks, MN (US); Lee Erik Swanson, Minneapolis, MN (US)

(73) Assignee: INSPIRE MEDICAL SYSTEMS, INC., Golden Valley, MN (US)

(21) Appl. No.: 18/281,087

(22) PCT Filed: Mar. 11, 2022
(86) PCT No.: PCT/US22/20034

§ 371 Date: Sep. 8, 2023

Related U.S. Application Data
(60) Provisional application No. 63/159,745, filed on Mar. 11, 2021.
(60) Provisional application No. 63/208,300, filed on Jun. 8, 2021.

(51) Int. Cl. A61N 1/36

(52) U.S. Cl. CPC A61N 1/3606; A61N 1/36135

(57) ABSTRACT

Systems and methods for treating bladder and/or bowel dysfunction of a patient includes a stimulation element implanted to stimulate one or more target sites, and a sensor to sense sensing at least one parameter of the patient indicative of a potential bladder or bowel dysfunction event. In some examples, stimulation energy is applied to an anatomical structure of the patient as a function of the sensed parameter, for example to address the potential bladder or bowel dysfunction event.

A portion of the population suffers from incontinence, such as one or both of urinary incontinence (or bladder incontinence) and fecal incontinence (or bowel incontinence). Diet, training, slings, and drug therapies may fail to treat incontinence.

### BRIEF DESCRIPTION OF DRAWINGS

FIG. 1 is a schematic illustration of anatomy of a human pelvic region.

FIG. 2 is a schematic illustration of the pelvic region of FIG. 1 and various nerves.

FIG. 3 is a block diagram of a treatment system in accordance with principles of the present disclosure.

FIGS. 4A-4C illustrate anatomy of a human pelvic region.

FIG. 4D is a simplified representation of a pudendal nerve relative to other anatomy of the pelvic region along with identifications of example stimulation target sites in accordance with principles of the present disclosure.

FIG. 4E is a representation of portions of the anatomy of the human pelvic region along with a lead assembly located in accordance with principles of the present disclosure relative to the perineal nerve branches of the pudendal nerve.

FIG. 4F is a representation of portions of the anatomy of the human pelvic region along with a portions of a treatment system, including an IPG implanted at an ischiorectal fossa in accordance with principles of the present disclosure.

FIG. 4G is another representation of the arrangement of FIG. 4F.

FIG. 5A is a diagram including a sectional view schematically representing a cuff electrode including circumferentially-arranged electrode contacts.

FIG. 5B is a diagram including a side view schematically representing a cuff electrode including circumferentially-arranged electrode contacts.

FIG. 6A is a simplified perspective view of a portion of a lead assembly useful with systems and methods of the present disclosure being applied to a nerve.

FIG. 6B is a simplified perspective view of the lead assembly of FIG. 6A upon final implant.

FIG. 7A is a simplified side view of a portion of a lead assembly useful with systems and methods of the present disclosure.

FIGS. 7B-7D are simplified cross-sectional views of electrode configurations useful with the lead assembly of FIG. 7A.

FIG. 8A is a simplified side view of a portion of a lead assembly useful with systems and methods of the present disclosure.

FIG. 8B is a simplified side view of a delivery tool useful with the lead assembly of FIG. 8A.

FIG. 9A is a simplified, side perspective view of a portion of lead assembly useful with the systems and methods of the present disclosure in a deployed state, along with a delivery sheath.

FIG. 9B is a simplified, side perspective view of the lead assembly and sheath of FIG. 9A arranged in a delivery state.

FIG. 10A is a simplified side view of a portion of a lead assembly useful with systems and methods of the present disclosure.

FIG. 10B is a simplified side view of a portion of a lead assembly useful with systems and methods of the present disclosure.

FIG. 11A is a simplified, side perspective view of a portion of lead assembly useful with the systems and methods of the present disclosure in a delivery state, along with a stylet.

FIG. 11B is a simplified, side perspective view of the lead assembly and stylet of FIG. 11A arranged in a deployed state.

FIG. 12A is a simplified, side perspective view of a portion of lead assembly useful with the systems and methods of the present disclosure in a delivery state, along with a sheath and a stylet.

FIG. 12B is a simplified, side perspective view of the lead assembly, sheath and stylet of FIG. 12A arranged in a deployed state.

FIG. 13A is a simplified, side perspective view of a portion of lead assembly useful with the systems and methods of the present disclosure in a delivery state, along with a stylet.

FIG. 13B is a simplified, side perspective view of the lead assembly and stylet of FIG. 13A arranged in a deployed state.

FIG. 14A is a simplified, side perspective view of a portion of lead assembly useful with the systems and methods of the present disclosure in a delivery state, along with a stylet.

FIG. 14B is a simplified, side perspective view of the lead assembly and stylet of FIG. 14A arranged in a deployed state.

FIG. 15 illustrates portions of the anatomy of the human pelvic region, including an obturator membrane.

FIG. 16 is a simplified perspective view of a portion of a lead assembly useful with systems and methods of the present disclosure.

FIG. 17A schematically illustrates a microstimulator useful with the systems and methods of the present disclosure.

FIG. 17B schematically illustrates a garment wearable by a patient in accordance with principles of the present disclosure and useful, for example, with the microstimulator of FIG. 17A.

FIG. 18 is a diagram identifying sensor types useful with the systems and methods of the present disclosure.

FIG. 19 is a diagram of an IPG, a stimulation lead, and a sensor lead in accordance with principles of the present disclosure.

FIG. 20 is a diagram of portions of the human anatomy including the bladder, and identifying possible implant locations of system components of the present disclosure.

FIG. 21 is a simplified side perspective view of a sensor assembly useful with systems and methods of the present disclosure implanted to a bladder.

FIG. 22 is a simplified perspective view of a portion of a lead assembly useful with systems and methods of the present disclosure implanted to a nerve.

FIGS. 23A and 23B are graphs illustrating algorithms useful with systems and methods of the present disclosure for estimating a potential leakage event.

FIGS. 24A-24E are graphs illustrating algorithms useful with systems and methods of the present disclosure for applying stimulation energy in the treatment of incontinence.

FIGS. 25A-25C are graphs illustrating algorithms useful with systems and methods of the present disclosure for applying stimulation energy in the treatment of incontinence.

FIGS. 26A-26C are graphs illustrating algorithms useful with systems and methods of the present disclosure for applying stimulation energy in the treatment of incontinence.

FIGS. 27A-27C are graphs illustrating algorithms useful with systems and methods of the present disclosure for applying stimulation energy in the treatment of incontinence.

FIGS. 28A-28C are graphs illustrating algorithms useful with systems and methods of the present disclosure for applying stimulation energy in the treatment of incontinence.

FIGS. 29A-29D are graphs illustrating algorithms useful with systems and methods of the present disclosure for applying stimulation energy in the treatment of incontinence.

FIG. 30 illustrates portions of an example trialing system in accordance with principles of the present disclosure as applied to a patient.

FIG. 31 is a simplified perspective view of a treatment system in accordance with principles of the present disclosure applied to anatomy of a patient.

FIG. 32 is a simplified perspective view of a treatment system in accordance with principles of the present disclosure applied to anatomy of a patient.

FIG. 33 is a simplified perspective view of a treatment system in accordance with principles of the present disclosure applied to anatomy of a patient.

FIG. 34 is a diagram identifying sensor types useful with the systems and methods of the present disclosure.

FIG. 35 is a graph illustrating representative signals generated by various sensors useful in monitoring a patient over time for possible event occurrence.

FIG. 36 is a graph illustrating processing of a raw accelerometer sensor signal over the time period of FIG. 35.

FIG. 37 is a graph illustrating representative raw magnitude signals from for accelerometers applied to a patient over the time period of FIG. 35.

FIG. 38 is a graph illustrating processing of a raw EMG sensor signal over the time period of FIG. 35.

FIG. 39 is a graph illustrating algorithms useful with systems and methods of the present disclosure for applying stimulation energy as a function of monitored sensor information.

FIG. 40 is a graph illustrating algorithms useful with systems and methods of the present disclosure for applying stimulation energy as a function of monitored sensor information.

FIG. 41 is a graph illustrating algorithms useful with systems and methods of the present disclosure for applying stimulation energy as a function of monitored sensor information.

FIG. 42A is a simplified perspective view of a treatment system in accordance with principles of the present disclosure applied to anatomy of a patient and reflecting method of the present disclosure.

FIG. 42B is an enlarged view of the portion “42B” of FIG. 42A.

FIG. 43 is a simplified perspective view of a treatment system in accordance with principles of the present disclosure applied to anatomy of a patient and reflecting method of the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to implantable devices for diagnosis, therapy, and/or other care of medical conditions. At least some examples may comprise implantable devices and/or methods of implanting devices useful for treating incontinence, including one or both of urinary incontinence and fecal incontinence of a patient, or other pelvic disorders. At least some such examples comprise implanting an electrode to deliver a nerve-stimulation signal to one or more nerves or nerve branches to activate a corresponding external sphincter, such as a branch of the pudendal nerve that activates the external urethral sphincter and/or the external anal sphincter. In some embodiments, operation of the implantable device is controlled in response to sensed information of the patient.

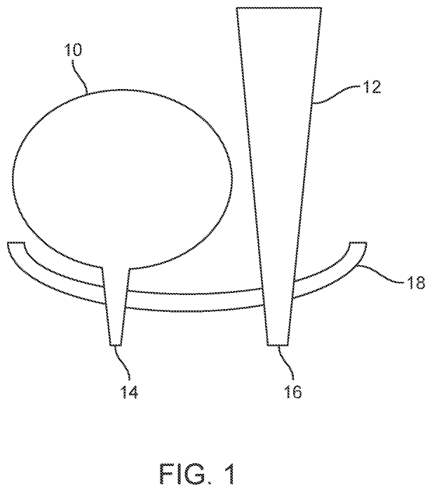

With reference to the greatly simplified view of FIG. 1, the human pelvic region includes a bladder 10 and a rectum 12. Contents of the bladder 10 are evacuated through a urethra 14, whereas contents of the rectum 12 are evacuated through anus 16. Pelvic floor muscles 18 support the pelvic organs and span the bottom of the pelvis. The pelvic floor muscle layer 18 has holes for passage of the urethra 14 and the anus 16, and normally wraps quite firmly around these holes to help keep the passages shut.

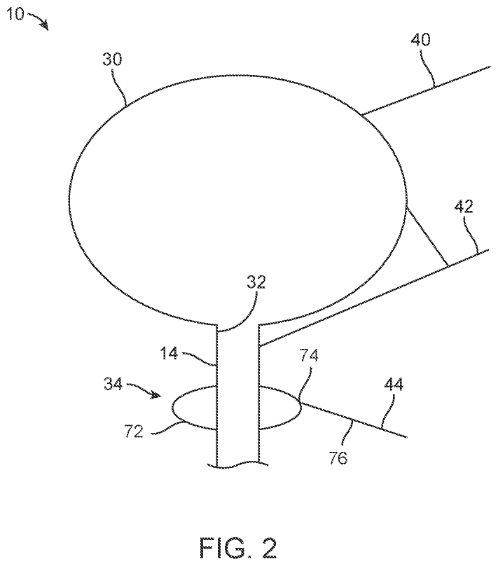

With additional references to the greatly simplified view of FIG. 2, the bladder 10 is a hollow muscular organ connected to the kidneys by the ureters. The detrusor 30 muscle (referenced generally) is smooth muscle found in the wall of the bladder 10. The urethra 14 is a tube or duct by which urine is conveyed out of the body from the bladder 10. Internal and external sphincters control flow of urine through the urethra 14; under normal conditions, when either of these muscles contracts, the urethra 14 is sealed shut. In particular, an internal urethral sphincter (IUS) 32 (referenced generally) is a smooth muscle that constricts the internal orifice of the urethra 14. The IUS 32 is located at the junction of the urethra 14 with the bladder 10 and is continuous with the detrusor muscle 30, but is anatomically and functionally fully independent from the detrusor muscle 30. An external urethral sphincter (EUS) 34 is located in the deep perineal pouch, at the bladder's 10 distal inferior end in females and inferior to the prostate in males. Urine is excreted from the kidneys and stored in the bladder 10 before elimination via the urethra 14 during what is known as the micturition reflex. During periods of bladder filling, the storage of urine is promoted by the actions of the internal and external urethral sphincters 32, 34 and the pelvic floor musculature 18. During micturition, these sphincters 32, 34 relax and the smooth muscle of the bladder (the detrusor muscle 30) contracts, resulting in the expulsion of urine.

The body of the bladder 10 is directly innervated by efferent fibers that arise from parasympathetic postganglionic neurons in the pelvic ganglia and intramural ganglia and by efferent fibers that arise from sympathetic postganglionic neurons in the lumbosacral sympathetic chain and hypogastric ganglia/pelvic ganglia. This is generally reflected in FIG. 2 by reference to a pelvic nerve 40 and a hypogastric nerve 42. The internal urethral sphincter 32 receives innervation from the hypogastric nerve 42. The external urethral sphincter 34 is directly innervated by motor neurons in the sacral segments of the spinal cord via the pudendal nerve 44.

Urinary continence is generally defined as the act of storing urine in the bladder 10 until the bladder 10 can be appropriately evacuated. Urinary continence requires control of the detrusor muscle 30 and is the result of complex coordination between multiple centers in the brain, brain stem, spinal cord, and peripheral nerves. As described above, micturition is a coordinated act of bladder elimination that involves relaxing the pelvic floor muscles 18, contracting the detrusor muscle 30, and simultaneously opening the urethral sphincters 32, 34 to achieve complete emptying of the bladder. Stress incontinence can be defined as the involuntary leakage of urine from the bladder 10 accompanying physical activity (e.g., laughing, coughing, sneezing, etc.) which places increased pressure on the abdomen. The leakage occurs even though the bladder muscles (detrusor muscle 30) is not contracting and an urge to urinate is not present. Stress incontinence can develop when the urethral sphincters 32, 34, the pelvic floor muscles 18, or all of these structures have been weakened or damaged and cannot dependably hold in urine. With urethral hypermobility, the bladder 10 and urethra 14 shift downward when abdominal pressure rises, and there is no hammock-like support for the urethra 14 to be compressed against to keep it closed. With urethral incompetence, problems in the urinary sphincter 32, 34 keep it from closing fully or allow it to pop open under pressure. Urinary urge incontinence (“UUI”) (sometimes referred to as overactive bladder (“OAB”) or detrusor overactivity) entails the involuntary leakage of urine from the bladder 10 when a sudden strong need to urinate is felt. There is a sudden involuntary contraction of the muscular wall (the detrusor 30) of the bladder that signals an immediate need to urinate, which can happen even when the bladder 10 is not full. Mixed incontinence is the term used to a combination of both overactive bladder and stress incontinence.

Internal and external sphincters are similarly provided with the anus 16 (i.e., the internal anal sphincter and the external anal sphincter), acting to keep the anal canal and orifice closed. Action of the internal anal sphincter (IAS) is entirely involuntary, and it is in a state of continuous maximal contraction. The external anal sphincter (EAS) is always in a state of contraction, but can be voluntarily put into a condition of greater contraction so as to more firmly occlude the anal orifice. Similar to urinary continence, bowel continence is the act of storing feces until an acceptable time and opportunity for elimination. Bowel continence requires competent internal and external sphincters, pelvic floor musculature, and intact neurological pathways. Neurological control of bowel continence is complex and requires coordinated reflex activities from the autonomic and enteric nervous systems. The colon can be visualized as a closed, pliant tube bounded by the ileocecal valve and the anal sphincter. The continuous, smooth muscle layer at the end of the rectum 12 thickens to form the internal anal sphincter (IAS); the external anal sphincter (EAS) is a circular band of striated muscle that contracts with the pelvic floor. Parasympathetic stimulation of the IAS from the pelvic plexus originates from the sacral cord (S1 to S2). Sympathetic stimulation of the IAS causes contraction. The EAS is composed of both smooth and striated muscle. The smooth muscle of the EAS is innervated by the enteric nervous system. The striated component of the EAS is innervated by the pudendal nerve that exits the cord at sacral levels S2, S3, and S4.

Fecal incontinence can be defined as the involuntary loss of rectal contents (feces, gas) through the anal canal and the inability to postpone an evacuation until socially convenient. For example, injuries to one or both of the EAS and IAS may make it difficult to hold stool back properly. Injury to the nerves that sense stool in the rectum or those that control the anal sphincter can also lead to fecal incontinence. A generalized weakness of the pelvic floor 18 can lead to an impaired barrier to stool in the rectum 12 entering the anal canal, and this is associated with incontinence to solids. The pelvic floor 18 is innervated by the pudendal nerve and the S3 and S4 branches of the pelvic plexus. If the pelvic floor muscles 18 lose their innervation, they cease to contract and their muscle fibers are in time replaced by fibrous tissue, which is associated with pelvic floor weakness and incontinence.

With the above in mind, some example systems and/or methods of the present disclosure relate to treating one or more of urinary incontinence, UUI and fecal incontinence by supplying stimulation signals to an electrode implanted to apply the stimulation signal to one or more nerves and/or muscles of the patient as described in greater detail below. In related systems and methods, monitoring, diagnosis and/or stimulation therapy can be implicated.

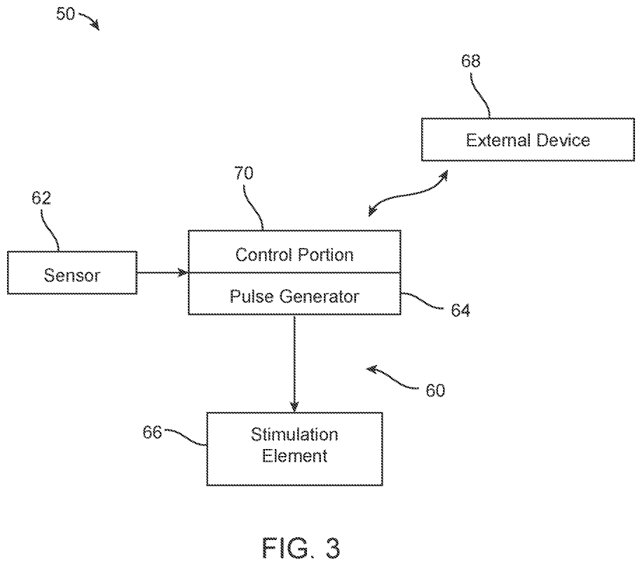

One example of a treatment system 50 in accordance with principles of the present disclosure is provided in FIG. 3 and includes an implantable medical device (IMD) 60 (referenced generally) and one or more sensors 62. Details on the various components are provided below. In general terms, the IMD 60 includes an implantable pulse generator (IPG) 64 and one or more stimulation elements (e.g., electrode or electrode assembly) 66. The IPG 64 is configured for implantation into a patient, and is configured to provide and/or assist in the performance of therapy to the patient. The stimulation element 66 is configured to be implanted proximate a selected segment or region of the patient's anatomy, and is electrically connected to the IPG 64. In other embodiments, the IPG 64 and the stimulation element 66 can be provided as components of a single or integral device, such as a microstimulator as are known in the art. The IPG 64 is programmed to deliver (or is prompted to deliver) stimulation signals to the stimulation element 66 that in turn apply the signal. In some embodiments, the IPG 64 is programmed (or is prompted) to initiate, cease and/or modulate (e.g., titrate) delivered stimulation signals based upon one or more physical parameters of the patient. In this regard, the sensor(s) 62 senses the physical parameter of interest, and signals the so-sensed parameter to the IPG 64 (or other component controlling operation of the IPG 64). The sensor 62 can be carried by the IPG 64, can be connected to the IPG 64, or can be a standalone component not physically connected to the IPG 64. The sensor 62 can be self-contained, and communicates with the IPG 64 in some optional embodiments. In some embodiments, the sensor 62, the IPG 64, and the stimulation element can be provided as components of a single or integral device. In some embodiments, the treatment system 50 can further include an optional external device 68. Where provided, the external device 68 can, in some non-limiting embodiments, wirelessly communicate with the IMD 60.

The IPG 64 can assume various forms known in the art for generating a nerve-stimulating signal for delivery to the stimulation element(s) 66. For example, the IPG 64 can include a sealed case or enclosure maintaining a power source (e.g., battery) and electrical/circuitry components appropriate for formatting energy from the power source as the desired stimulation signal (e.g., a nerve-stimulation signal). In some embodiments, the IPG 64 as provided as part of, or is electronically linked to, a control system that includes a control portion 70 providing one example implementation of a control portion forming a part of, implementing, and/or generally managing stimulation element(s), power/control elements (e.g. pulse generators, microstimulators), sensors, and related elements, devices, user interfaces, instructions, information, engines, elements, functions, actions, and/or methods, as described throughout examples of the present disclosure. In some examples, the control portion 70 includes a controller and a memory. In general terms, the controller comprises at least one processor and associated memories. The controller is electrically couplable to, and in communication with, memory to generate control signals to direct operation of at least some of the stimulation elements, power/control elements (e.g. pulse generators, microstimulators) sensors, and related elements, devices, user interfaces, instructions, information, engines, elements, functions, actions, and/or methods, as described throughout examples of the present disclosure. In some non-limiting examples, these generated control signals include, but are not limited to, employing instructions and/or information stored in the memory to at least direct and manage treatment of incontinence by stimulating nerve(s), nerve branch(es) and/or muscle(s) to activate one or more of the external urethral sphincter 34 and the external anal sphincter, and/or pelvic floor nerves (e.g., the pudendal nerve 44, the sacral nerve) to relax the detrusor muscle 30 and prevent or reduce urgency or frequency. In some instances, the controller or control portion 70 may sometimes be referred to as being programmed to perform the actions, functions, routines, etc. of the present disclosure. In some examples, at least some of the stored instructions are implemented as, or may be referred to as, a care engine, a sensing engine, monitoring engine, and/or treatment engine. In some examples, at least some of the stored instructions and/or information may form at least part of, and/or, may be referred to as a care engine, sensing engine, monitoring engine, and/or treatment engine.

In response to or based upon commands received via a user interface and/or via machine readable instructions, the controller generates control signals as described above in accordance with at least some of the examples of the present disclosure. In some examples, the controller is embodied in a general purpose computing device while in some examples, the controller is incorporated into or associated with at least some of the stimulation elements, power/control elements (e.g. pulse generators, microstimulators), sensors, and related elements, devices, user interfaces, instructions, information, engines, functions, actions, and/or method, etc. as described throughout examples of the present disclosure.

For purposes of the present disclosure, in reference to the controller, the term “processor” shall mean a presently developed or future developed processor (or processing resources) that executes machine readable instructions contained in a memory. In some examples, execution of the machine readable instructions, such as those provided via the memory of the control portion 70 cause the processor to perform the above-identified actions, such as operating the controller to implement the sensing, monitoring, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by the memory. In some examples, the machine readable instructions may comprise a sequence of instructions, a processor-executable machine learning model, or the like. In some examples, the memory comprises a computer readable tangible medium providing non-volatile storage of the machine readable instructions executable by a process of the controller. In some examples, the computer readable tangible medium may sometimes be referred to as, and/or comprise at least a portion of, a computer program product. In other examples, hard wired circuitry may be used in place of or in combination with machine readable instructions to implement the functions described. For example, the controller may be embodied as part of at least one application-specific integrated circuit (ASIC), at least one field-programmable gate array (FPGA), and/or the like. In at least some examples, the controller is not limited to any specific combination of hardware circuitry and machine readable instructions, nor limited to any particular source for the machine readable instructions executed by the controller.

In some examples, the control portion 70 may be entirely implemented within or by a stand-alone device.

In some examples, the control portion 70 may be partially implemented in the IPG 64 and partially implemented in a computing resource separate from, and independent of, the IPG 64. For instance, in some examples the control portion 70 may be implemented via a server accessible via the cloud and/or other network pathways. In some examples, the control portion 70 may be distributed or apportioned among multiple devices or resources such as among a server, a congestive heart failure treatment device (or portion thereof), and/or a user interface.

In some examples, the control portion 70 is entirely implemented within or by the IPG 64 (thereby defining an IPG assembly), which has at least some of substantially the same features and attributes as a pulse generator (e.g. power/control element, microstimulator) as previously described throughout the present disclosure. In some examples, the control portion 70 is entirely implemented within or by a remote control (e.g. a programmer) external to the patient's body, such as a patient control and/or a physician control (e.g., the external device 68). In some examples, the control portion 70 is partially implemented in the IPG 64 assembly and partially implemented in the remote control (at least one of the patient control and the physician control).

### Stimulation Target Sites

With reference between FIGS. 1-3, as described in greater detail below, the system 50 can be configured and implanted to provide stimulation therapy to one or more nerves and/or muscles that, for example, influence the behavior of musculature of the pelvic region of the patient, for example musculature relating to one or both of urinary incontinence and fecal incontinence (e.g., the external urethral sphincter 34, the internal urethral sphincter 32, pelvic floor muscles 18, the external anal sphincter, the internal anal sphincter, etc.). For example, stimulation can be provided to one or more of the pudendal nerve 44, the pelvic nerve 40, the sacral nerve, or branches thereof. Alternatively or in addition, the system 50 can apply electrical stimulation to tissue sites proximate a nerve or nerve branch of interest. In yet other embodiments, stimulation can be applied directly to a muscle.

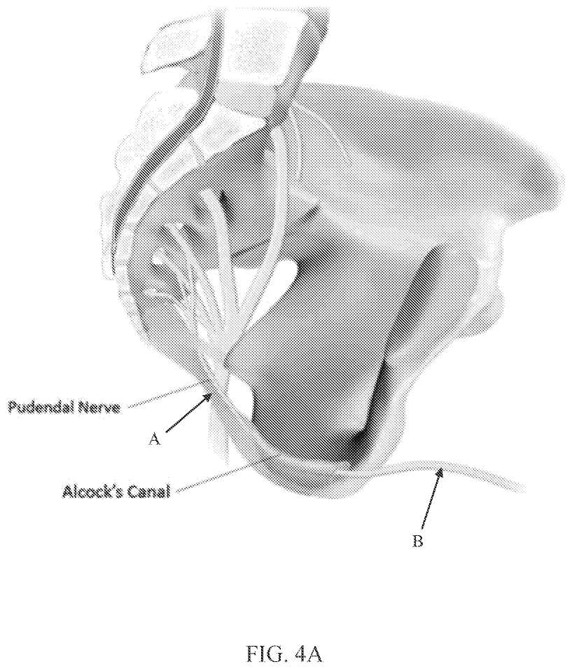

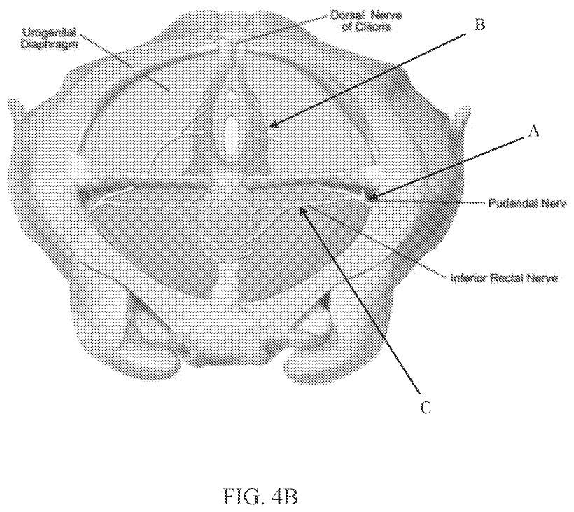

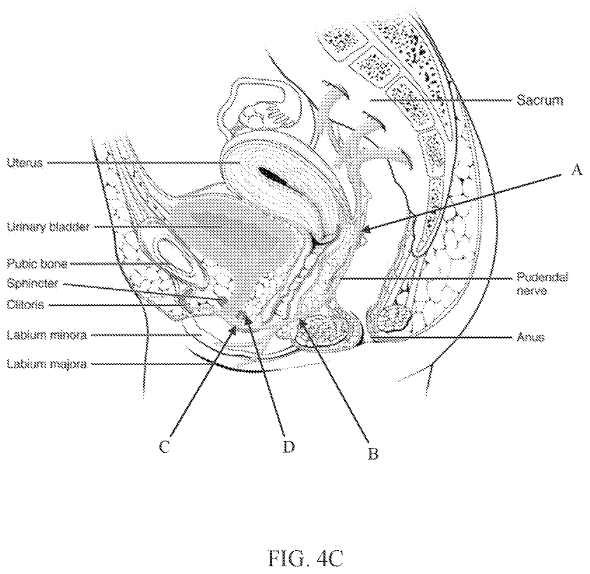

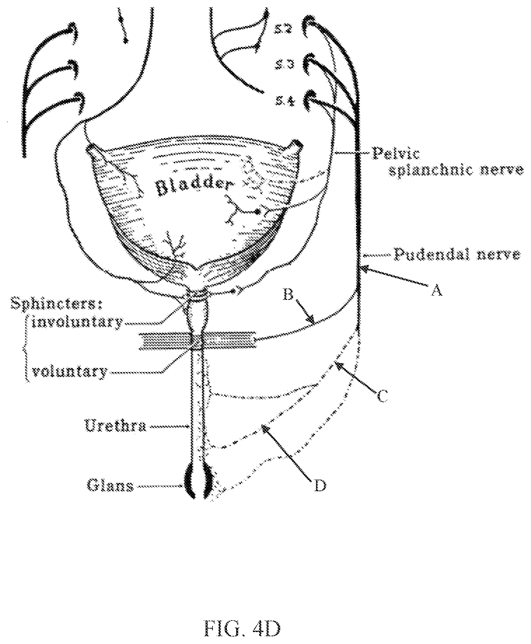

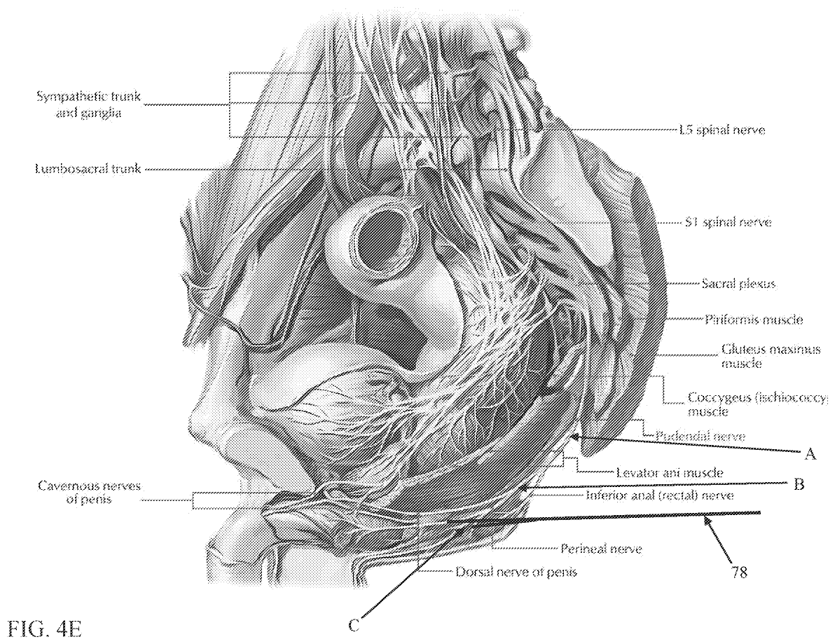

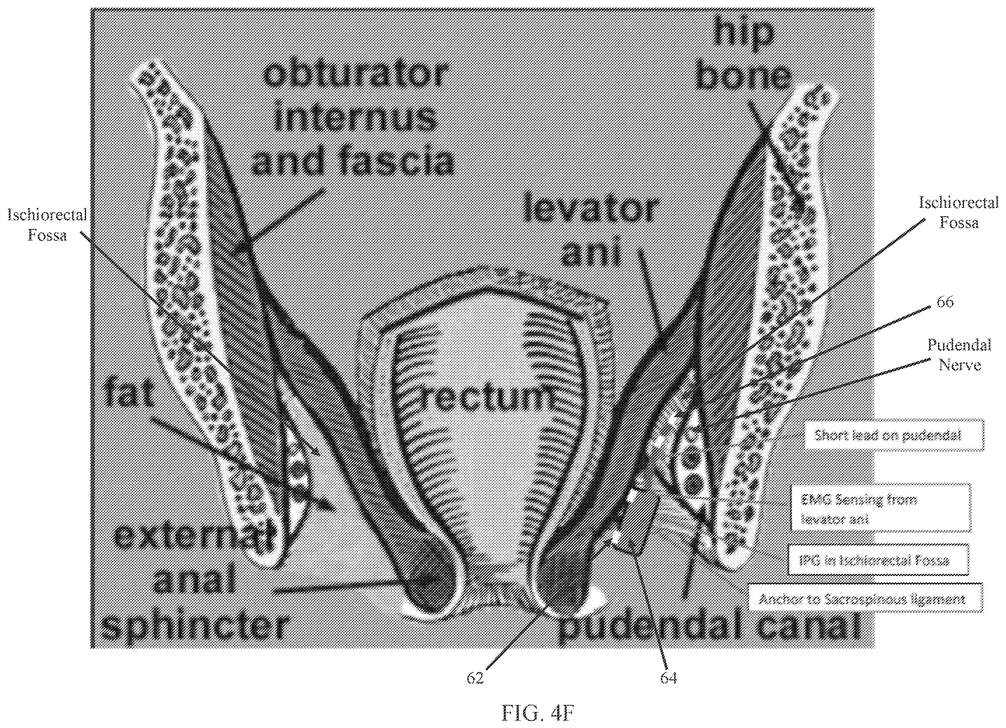

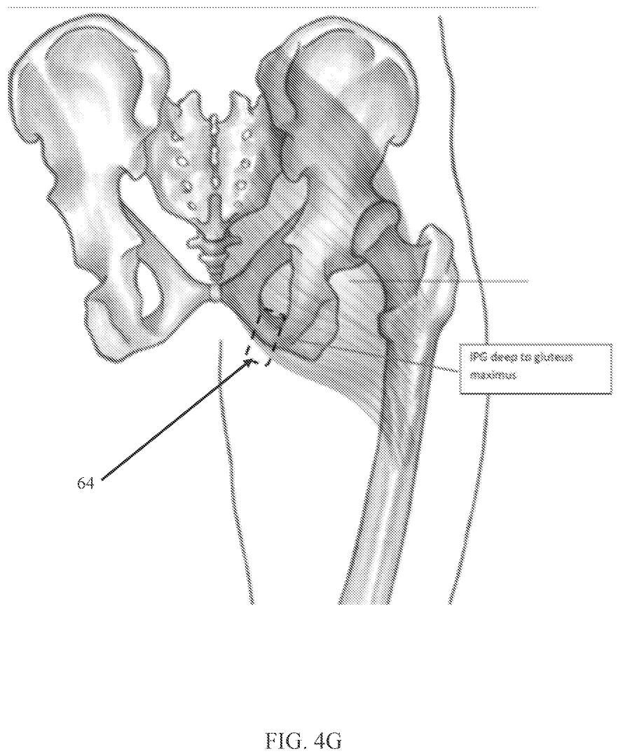

With embodiments in which stimulation is to be applied to the pudendal nerve 44, various specific targets sites can be employed. As a point of reference, the pudendal nerve 44 is found in the pelvis, and is the main nerve of the perineum. With additional reference to FIGS. 4A-4E, the pudendal nerve 44 is formed as three roots immediately converge above the upper border of the sacrotuberous ligament and the coccygeus muscle. The three roots become two cords when the middle and lower root join to form the lower cord, and these in turn unite to form the pudendal nerve proper just proximal to the sacrospinous ligament. The pudendal nerve 44 passes into Alcock's canal (also known as the pudendal canal); inside the canal, the nerve divides into branches, including the inferior rectal nerve, the perineal nerve and the dorsal nerve. The perineal nerve branch of the pudendal nerve further divides into the deep (muscular) and superficial (cutaneous) perineal nerve branches. With this anatomy in mind, in some examples, systems and methods of the present disclosure can include applying stimulation to a posterior region of the pudendal nerve 44 (e.g., targeting the pudendal nerve 44 posterior to where it enters Alcock's canal, for example by delivering a lead or the like through the buttocks). In other examples, systems and methods of the present disclosure are configured to apply stimulation to the perineal branch of the pudendal nerve 44 (e.g., targeting the terminal perineal branches of the pudendal nerve where they innervate the external urethral sphincter). Various target sites of the pudendal nerve 44 in accordance with non-limiting examples of the present disclosure are labeled at A, B, C and D in FIGS. 4A-4E. One example of a possible lead 78 configured for stimulating two (or more) terminal perineal branches of the pudendal nerve is shown in FIG. 4E; as shown, the lead 80 can have a bifurcated or Y-shape, locating a stimulating element carried by each arm of the lead at a different terminal perineal branch.

As a point of reference, some benefits of functional stimulation (e.g., at levels sufficient to cause muscular contraction) of the pudendal nerve base or posterior pudendal nerve (proximal the pudendal nerve branches, such as location A in FIGS. 4A-4E) can be to achieve a broader reaction to the pelvic floor that could, in turn, help to prevent a leakage event through direct activation of the urethral sphincter, but also by providing support to the bladder, the urethra and involuntary structures. Functional stimulation at this location is also likely to have beneficial effects for bowel incontinence through a similar combination of mechanisms. Sub-functional stimulation as this location has also been shown to have beneficial effects for OAB and bowel incontinence (e.g., non-stress or functional conditions) when stimulated at sub-functional levels. Functional stimulation of the pudendal branches that directly innervate the urethral sphincter muscles (e.g., location B, C or D in FIGS. 4A-4E) can also be effective at preventing leak events, however it will have a more localized effect. As compared to stimulation of the pudendal nerve base, functional stimulation of the pudendal nerve branches that directly innervate the urethral sphincter muscles may be less effective (though perhaps more comfortable) in some patients because it lacks the broader supportive effects functional stimulation more proximal on the pudendal nerve. Further, stimulation of the pudendal nerve branches that directly innervate the urethral sphincter muscles is not likely to affect bowel incontinence, and is likely to have more limited impact at sub-functional stimulation levels.

A stimulation location or target site of the present disclosure along the posterior pudendal nerve (e.g., location A in FIGS. 4A-4E) can represent, in some embodiments, a proven access point for a minimally invasive, soft-tissue anchor approach. Stimulation or activation along the posterior pudendal nerve can recruit much of the pelvic floor, providing, for example, urinary stress incontinence and bowel treatment/therapy as well as supporting the pelvic organs to increase the therapeutic effect. In other embodiments, a posterior pudendal nerve target site can serve as an effective location for OAB therapy (e.g., sub-muscle recruitment stimulation).

A stimulation location or target site of the present disclosure along the central pudendal nerve (e.g., location B in FIG. 4A, location A in FIG. 4B, location B in FIG. 4C, location C (plus branch to the right of location C) in FIG. 4D, location B in FIG. 4E) can exclude rectal branches of the pudendal nerve while providing other benefits of a posterior placement/approach.

A stimulation location or target site of the present disclosure along the distal pudendal nerve, perineal nerve, nerve roots of the urethral sphincter, or urethral sphincter muscle (e.g., location B in FIG. 4B, location C/D in FIG. 4C, location D in FIG. 4D, location C in FIG. 4E) can provide a more focused effect on urinary stress incontinence, and can be less likely to have other side effects from stimulation. These target sites (as compared to other target sites mentioned above) may present a more challenging location for electrode placement, and may be less likely to be effective for treatment of OAB and fecal incontinence.

A stimulation location or target site of the present disclosure along the rectal pudendal nerve can provide a focused effect for fecal incontinence. These target sites may present a challenging implant location.

Additional example implant locations and approaches are implicated by the views of FIGS. 4F and 4G, and in particular the ischiorectal fossa. The ischiorectal fossa is the space that exists between the internal surface of the perineal skin and the plane of the plate of the levator ani muscle. The space is pyramidal, with its base directed to the surface of the perineum and its apex directed anteromedially toward the pubic symphysis. As a point of reference, in the coronal sectional view of FIG. 4F, the right ischiorectal fossa (left side of the orientation of FIG. 4F) is shown/labeled with naturally occurring fatty tissue, whereas the fatty tissue of the left ischiorectal fossa (right side of the orientation of FIG. 4F) has been removed for ease of understanding. With this in mind, in some embodiments of the present disclosure, the IPG 64 can be implanted in the ischiorectal fossa, anchored, for example, to the sacrospinous ligament (small or anterior sacrosciatic ligament), the sacrotuberous ligament, or other connective tissue. The IPG 64 can have a size and shape or footprint conducive to placement within the space of the ischiorectal fossa, and thus may be smaller than IPG formats conventionally implanted at other bodily locations (e.g., the IPG 64 format for an ischiorectal fossa implant location can be considered a “miniature” IPG). With these and other embodiments, the stimulation element(s) 66 can be provided as part of a short lead extending from the IPG 64 and located to deliver stimulation energy to the pudendal nerve at or near the Alcock's canal for example. Other stimulation target sites can also be implicated by the lead. Alternatively or in addition, stimulation element(s) can be provided on housing or “can” of the IPG 64 and otherwise arranged, upon final implant of the IPG 64, to direct stimulation energy to the pudendal nerve (or other targeted nerve or other tissue).

One or more sensors can be provided with the arrangement of FIG. 4F. In some non-limiting examples, one or more of the sensors 62 can be carried by the housing or “can” of the IPG 64 as shown. With the IPG implant location of FIG. 4F, the sensor(s) 62 carried by the IPG housing can naturally be in close proximity, or contact, the levator ani muscle (pelvic floor). With these and related embodiments, the sensor(s) 62 can be, or can be akin to, an EMG sensor, thus sensing changes in the pelvic floor (that in turn are indicative of abdominal pressure). Alternatively or in addition, the sensor(s) 62 can be, or can be akin to, a movement sensor, a pressure sensor, strain gauge or the like, appropriate for sensing or detecting activity at the levator ani or pelvic floor. In yet other embodiments, a sensor-carrying lead can extend from the IPG 64 that protrudes into or through the levator ani or other structures of the pelvic floor for sensing parameters at an opposite side of the levator ani for more direct sensing of intraabdominal pressure.

FIG. 4G further illustrates a location of the implanted IPG 64 of the arrangement of FIG. 4F relative to other anatomy. As shown, the IPG 64 is implanted or located “under” or deep to the gluteus maximus. These and other implant locations can be conducive to straightforward and convenient recharging of a battery of the implanted IPG 64, for example by the patient simply sitting on a support (e.g., a chair) that otherwise carries the transmission coil and other components of the charging system.

The IPG 64 can be implanted to the ischiorectal fossa locations implicated by FIGS. 4F and 4G in various manners. In some non-limiting examples, laparoscopic techniques/tools can be employed. Unlike other implant locations in which an invasive, percutaneous introducer needle stick/puncture thorough the gluteus maximus muscle followed by tunneling may be required, the ischiorectal fossa target site of some examples of the present disclosure is readily accessible via a laparoscopic approach (e.g., incision made inferior to the gluteus maximus); muscle cutting and tunneling are not necessary in some embodiments, promoting a faster healing time for the patient. Furthermore, access to the pudendal nerve and its branches in the ischiorectal fossa could allow selective intraoperative stimulation using nerve monitoring methods to determine the most effective stimulation location to maximize the evoked response to improve continence and minimize or prevent unwanted sensations from electrical stimulation.

Returning to FIGS. 1-3, the stimulation element(s) 66 can be provided as part of stimulation lead or lead assembly or the like. With these and related embodiments, the lead can be delivered in various manners to position the stimulation element(s) 66 at an intended target site, for example along the pudendal nerve 44. For example, the lead can be delivered using techniques conventionally employed for a pudendal nerve block. In other embodiments, the lead can be delivered posteriorly (e.g., through buttocks), anteriorly, via a transvaginal approach, via a trans-urethral approach, etc.

In yet other embodiments, stimulation energy can be applied to one or more other nerves implicating bladder and/or anus control. As a point of reference, various nerves relevant to urinary continence and/or micturition include the pudendal nerve 44, pelvic nerve 40, and hypogastric nerve 42. The hypogastric nerve 42 is part of the sympathetic nervous system, and can inhibit contraction of the detrusor muscle as well as activate or contract the muscles of the urethra (and the neck of the bladder). With some embodiments of the present disclosure, stimulation energy is applied to the hypogastric nerve(s) 42 at the S, T or L level (e.g., sympathetic nerves T10-L2) in a manner that encourages the body's natural, unconscious or reflexive control over voiding, for example to prevent leakage. In other embodiments, stimulation energy is applied to the pelvic splanchnic nerves (or other parasympathetic nerve implicating urinary or fecal continence) at the S, T or L level (from T11, T12-L1, L2) in a manner that suppresses parasympathetic nerve impulses otherwise “activating” the patient's normal micturition drive; this, in turn, can enhance continence by allowing greater sympathetic nerve activity or through a relax action that has similar benefit.

In some non-limiting embodiments, the external urethral sphincter 34 is the structure targeted to be affected by the systems and methods of the present disclosure. In addition or alternatively, the internal urethral sphincter 32 can be targeted for contraction with the systems and methods of the present disclosure, for example by direct muscle stimulation, stimulation of the pelvic or hypogastric nerves, etc. In yet other embodiments, the systems and methods of the present disclosure can apply stimulation energy to affect one or more pelvic floor muscles 18 (or other structures of the pelvic floor) that implicate continence, such as the levator ani, the compressor urethrae, the urethrovaginal sphincter, the bulbospongiosus, the pubovaginalis, etc.

As is evident from the descriptions of the present disclosure, with some example systems and methods of the present disclosure, stimulation energy can be applied to one, two, three or more target sites, and information can be sensed from one, two, or more target sites. In some non-limiting embodiments, a first stimulation element is located to apply stimulation energy intended to activate the external urethral sphincter 34 and a second stimulation element is located to apply stimulation energy intended to activate the external anal sphincter (so as to treat both urinary and fecal incontinence). In other embodiments, the second stimulation element can be located to activate the pelvic floor muscles 18 (e.g., to elevate the bladder 10 of a patient suffering from bladder prolapse). The first and second stimulation elements can be driven by the same IPG 64, or two (or more) IPG's can be provided. With some systems and methods of the present disclosure, stimulation elements are located to stimulate at least two, optionally all, of the hypogastric nerve 42, pelvic nerve 40, and pudendal nerve 44.

Regardless, in some embodiments, the delivered electrical stimulation modulates muscle activity to treat, for example, stress incontinence, UUI and/or mixed incontinence.

### Stimulation Elements and Lead Assemblies

The stimulation element 66 can assume various forms appropriate for applying electrical stimulation to the anatomical feature (e.g., nerve) of interest. For example, the stimulation element 66 can be formatted for targeting a sacral nerve via the sacral foramen (e.g., configured to be delivered percutaneously, optionally with fluoroscopy support). The stimulation element 66 can be or include one or more electrodes in the form of ring electrodes, segmented electrodes, and partial ring electrodes. In some examples, the example stimulation element(s) may be or include a cuff electrode, comprising at least some of substantially the same features and attributes as described in Bonde et al., U.S. Pat. No. 8,340,785, Self Expanding Electrode Cuff, issued on Dec. 25, 2102 and Bonde et al., U.S. Pat. No. 9,227,053, Self Expanding Electrode Cuff, issued on Jan. 5, 2016, both which are hereby incorporated by reference in their entirety. Moreover, in some examples a stimulation lead, which may comprise one example implementation of a stimulation element, may comprise at least some of substantially the same features and attributes as the stimulation lead described in U.S. Pat. No. 6,572,543 to Christopherson et al., and which is incorporated herein by reference in its entirety.

With optional embodiments in which the stimulation element 66 is or includes a cuff-type format, the stimulation element 66 can be configured to target nerves controlling the external urethral sphincter 34 and/or the external anal sphincter. In some embodiments, the stimulation element 66 is configured to target the external urethral sphincter 34 muscle directly, and can be delivered trans-urethrally in accordance with some methods of the present disclosure. With these and related embodiments, a design or form factor of the stimulation element 66 can be customized for the size of the external urethral sphincter 34 of an individual patient. The cuff format can wrap about the external urethral sphincter 34 muscle and then cause contraction thereof with electrical stimulation. Wrapping about the external urethral sphincter 34 muscle (and thus the urethra 14) can provide a highly viable target site that better ensures that terminal nerve branches will be available for stimulation. With these and other embodiments, the stimulation element 66 can be designed to include or incorporate an active and/or passive anchoring system (e.g., sutures, tines, etc.). With some optional systems and methods of the present disclosure, the stimulation element 66 is or includes a cuff electrode applying stimulation energy to a targeted nerve and affixed to the muscle at a location at which the targeted nerve innervates the muscle. With other optional systems and methods of the present disclosure, two or more cuff-type electrodes are provided, each electrically connected to the same IPG 64 (e.g., each of the two or more cuff-type electrodes are carried by a common lead body, or two or more of the cuff-type electrodes can be carried by separate lead bodies that are each connected to a separate port in the header of the IPG 64). With these and related embodiments, one cuff-type electrode can be implanted to affect or stimulate a first nerve and a second cuff-type electrode can be implanted to affect or stimulate a second nerve; alternatively, two or more cuff-type electrodes can be implanted along the same nerve, but at different branches thereof (e.g., upon final implant, the system can operate to stimulate multiple branches of the pudendal nerve with different cuff-type electrodes). In yet other embodiments, two or more non-cuff-type leads can be utilized, or a combination of one or more cuff-type leads and one or more non-cuff-type leads.

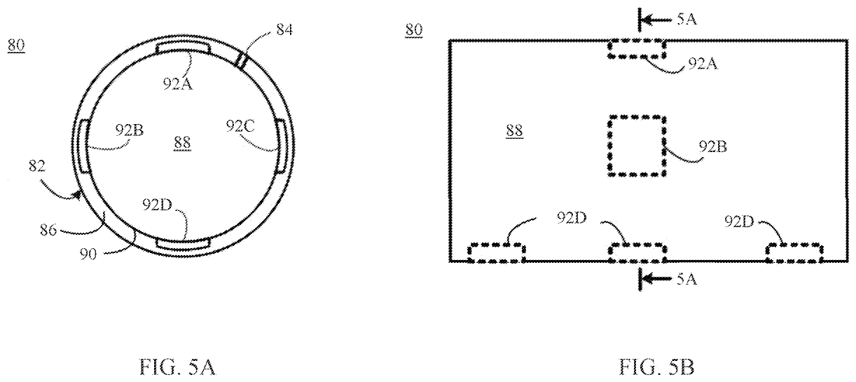

FIG. 5A is a side view, and FIG. 5B is a side view, schematically representing an example arrangement including a cuff electrode 80. In some examples, the cuff electrode 80 in FIGS. 5A-5B may comprise at least some of substantially the same features and attributes as the stimulation element(s) of other embodiments of the present disclosure. In some examples, the cuff electrode 80 comprises a generally cylindrically-shaped cuff body 82 for encircling a periphery of a nerve and including a slit 84 in a wall 86 of the cuff body 82 to permit opening and mounting of the cuff body 82 onto an encircling a nerve, which would extend within and through a lumen 88 defined by an interior surface 90 of the cuff body 82. As further shown in FIG. 5A, in some examples the cuff electrode 80 may comprise a bottom row of axially spaced apart electrodes 92D and a middle row of circumferentially spaced apart electrodes 92A, 92B, 92D, 92C. By employing various combinations of the respective electrodes 92A, 92B, 92C, 92D, as well as variations in the stimulation signal as previously described, this electrode configuration may be used to provide selective stimulation and/or stimulation steering of a stimulation signal relative to different fascicles, nerve fibers, etc. within a nerve about which the cuff electrode 80 is secured. In some examples, such selective stimulation may be used to manage stimulation dosing to mitigate potential adaptation of an overall blood pressure/cardiovascular system to the stimulation.

Returning to FIGS. 1-3, other implantable stimulation element or electrode configurations appropriate for applying stimulation energy to a selected region or segment are also acceptable. For example, the stimulation element 66 can be provided as part of an electrode assembly configured to wrap partially or completely about the selected region or segment of a targeted nerve or anatomical feature. In other embodiments, the stimulation element 66 can be provided as part of an electrode assembly carrying a tissue fastener or fixation unit or anchor (e.g., a screw or similar mechanical coupling device or mechanism) formatted to be inserted or secured to tissue highly proximate the targeted nerve, nerve branch, or muscle. Any of the stimulation element embodiments of the present disclosure (e.g., cuff electrode, lead assembly, microstimulator, etc.) can be secured or anchored to tissue at various locations, such as muscle/soft tissue, bone, obturator membrane, etc.

With some example systems and methods of the present disclosure, the stimulation element 66 is placed on an external plane of a targeted muscle to apply electrical stimulation to the targeted muscle and/or to a targeted nerve that innervates the muscle at the location of implant. For example, the stimulation element 66 can be delivered through a wall of the bladder 10, through the skin and onto the bladder 10 via an access location below the lowest rib.

In some embodiments, the stimulation element 66 is provided as an array of electrode contacts, with the electrodes of the array being selectively activated to produce a desired stimulation vector. For example, the electrode array can be a 3×3 array of electrode contacts, a 3×4 array of electrode contacts, etc. Each given stimulation element may comprise an array of electrically conductive elements (e.g. electrodes, electrode contacts, etc.), which may be arranged in in a wide variety of configurations, such as but not limited to a row, rows, staggered configurations, grid (2×2, 3×3), and combinations thereof. As is known in the art, different combinations of the electrode contacts can be activated. In another example, the stimulation element 66 can be provided as one of a series of ring electrodes spaced along a lead, with each of the ring electrodes being secured over the target site at spaced locations.

In some examples, the stimulation element(s) 66 can be electrically connected relative to a common element, such as the IPG 64 with such connective wires omitted for illustrative clarity or with such connection being wireless. In some instances, the example of connective wires may take the form of a lead for the stimulation element 66.

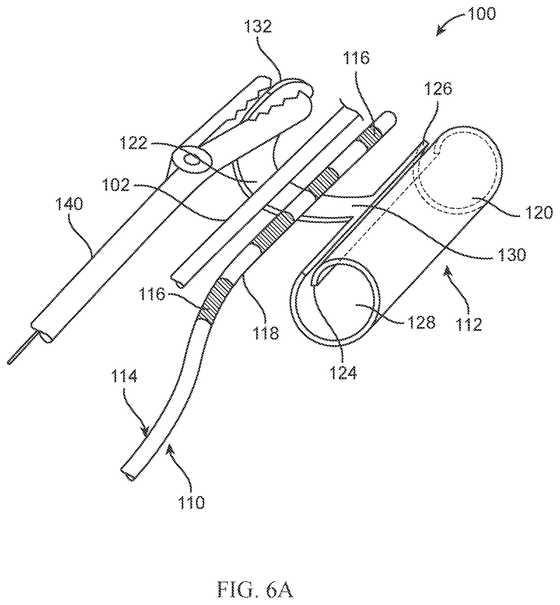

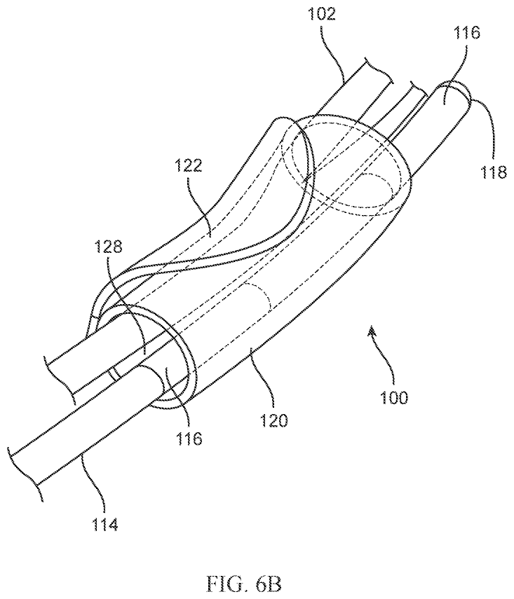

In some embodiments, the stimulation element(s) 66 is provide as part of an electrode lead assembly adapted, for example, to be implanted percutaneously (e.g., via a laparoscopic approach). For example, FIG. 6A illustrates one non-limiting example of an electrode lead assembly 100 of the present disclosure being secured or implanted relative to a nerve 102 (e.g., the pudendal nerve). The lead assembly 100 includes a lead 110 and a cuff 112. The lead 110 can have a format and construction akin to conventional lead designs, including a lead body 114 carrying one or more stimulation elements or electrodes 116 (e.g., ring electrodes) along a distal region 118 thereof. Electrical connections or wiring (not shown) for each of the electrodes 116 is carried by lead body 114 in an electrically isolated fashion, with a proximal region (not shown) of the lead body 114 formatted for physical and electrical connection to an implantable pulse generator. Other lead constructions are also acceptable.

The cuff 112 includes a cuff body 120 and a strap 122. The cuff body 120 is formed of a soft, electrically non-conductive material appropriate for direct contact with a nerve; for example, the cuff body 120 can be silicone. The cuff body 120 is configured to self-assume or self-revert to the overlapping or wrapped shape reflected by FIG. 6A. The wrapped shape can include a first edge 124 located below or inside of an opposing second edge 126, with the cuff body 120 forming or defining a central passage 128. The passage 128 can be accessed via a gap between the opposing edges 124, 126. A fixed end 130 of the strap 122 is attached or connected to the cuff body 120 (e.g., at or adjacent the second edge 126), with the strap 122 extending from the cuff body 120 to a free end 132. The strap 122 is configured to self-assume or self-revert to a curved shape, effectively wrapping over or along an exterior of the cuff body 120 in a normal state (it being understood that FIG. 6A reflects the strap 122 in a deflected state). The strap 122 can be integrally formed with the cuff body 120, or can be separately formed and assembled to the cuff body 120. Regardless, the free end 132 of the strap 122 can be manipulated or deflected away from the cuff body 120 as shown in FIG. 6A, affording more direct access to, and perhaps enlarging, the gap between the cuff body edges 124, 126.

During an implantation procedure, the distal region 118 of the lead body 114 is delivered to the target site as shown, locating the electrodes 116 along or in close proximity to the nerve 102 of interest. The cuff 112 is similarly delivered to the target site (e.g., via a laparoscopic scope). The clinician uses a tool 140 (e.g., a laparoscopic grasper) to engage the strap 122 and manipulate the free end 132 away from the cuff body 120 as shown. The lead 110 and the nerve 102 can then readily be inserted between the cuff body edges 124, 126, and into the central passage 128. The strap 122 is then released from the tool 140, and self-reverts to a normal state in which the strap 122 wraps about the cuff body 120. FIG. 6B illustrates the lead assembly 100 upon final implant. The distal region 118 of the lead body 114 (and thus the electrodes 116) is retained against the nerve 102 within the central passage 128 (referenced generally), with the natural or normal overlapping or wrapped shape of the cuff body 120 and the strap 122 preventing the lead body 114 from becoming displaced relative to the nerve 102.

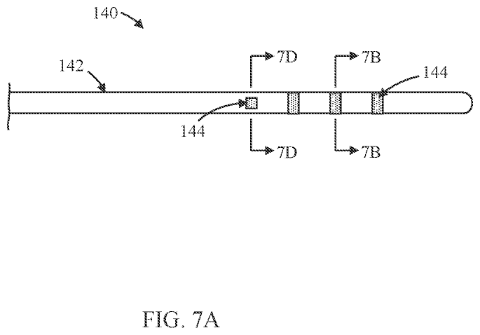

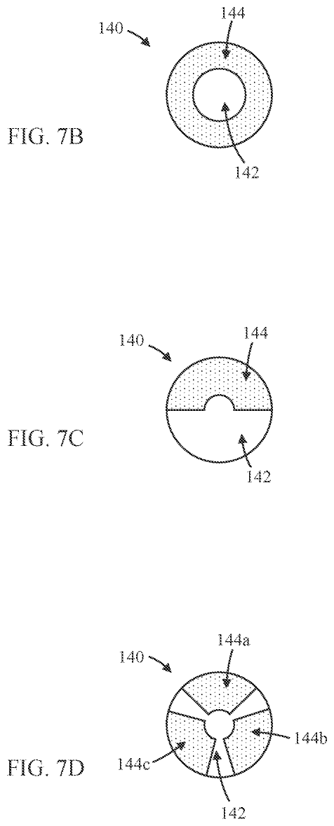

Some of the example stimulation element/lead assemblies of the present disclosure can assume other forms appropriate for percutaneous delivery/implantation to a location conducive to stimulating a desired nerve segment (e.g., the pudendal nerve) or other anatomy (e.g., direct muscle stimulation). For example, the percutaneous lead bodies of the present disclosure can optionally have an elongated, generally cylindrical shape and carry one or more stimulation elements (e.g., ring electrodes, partial ring electrodes, segmented electrodes, etc.) at a distal region thereof. By way of example, FIG. 7A illustrates portions of a lead assembly 140 useful with embodiments of the present disclosure and including a lead body 142 and one or more stimulation elements 144. The lead body 142 can have a generally cylindrical shape and construction as is known in the art. Any number of stimulation elements 144 can be carried at a distal region of the lead body 142 (thus, the example of FIG. 7A in which four of the stimulation elements 144 are shown is in no way limiting). Where two or more of the stimulation elements 144 are provided, they can have similar or dissimilar constructions. For example, FIG. 7B reflects the stimulation element 144 having a ring-like shape (e.g., a ring electrode as is known in the art). Alternatively, one or more or all of the stimulation elements 144 can be, or can be akin to, a partial ring electrode as represented by FIG. 7C. In other embodiments, one or more or all of the stimulation elements 144 can be, or can be provided as, segmented electrodes. A segmented electrode can be considered as an electrode of a group of electrodes that are positioned at the same longitudinal location along the longitudinal axis of the lead body 142 as reflected by the example segmented electrodes 144a-144c of FIG. 7D. While three of the segmented electrodes 144a-144c are shown, any other number is also acceptable. In some embodiments, 2-4 radially segmented circumferential electrodes can be beneficial. Further, some of the percutaneous lead assemblies of the present disclosure are configured to provide one or more of deployable fixation features, reversible (e.g., re-sheathable) fixation features, and bilateral stability fixation features. With these and related embodiments, the optional lead assemblies of the present disclosure can be useful, for example, with percutaneous delivery techniques for accessing and applying stimulating to the pudendal nerve. As a point of reference, the need for fixation of a percutaneously-delivered stimulation lead relative to the pudendal nerve can be different from that associated with a sacral foramen nerve. For example, a sacral foramen nerve stimulation lead assembly normally accounts for only retrograde expulsion (although antegrade migration may occasionally occur), whereas a percutaneous pudendal nerve stimulation lead assembly desirably provides bi-lateral fixation (resisting both retrograde and antegrade migration/expulsion).

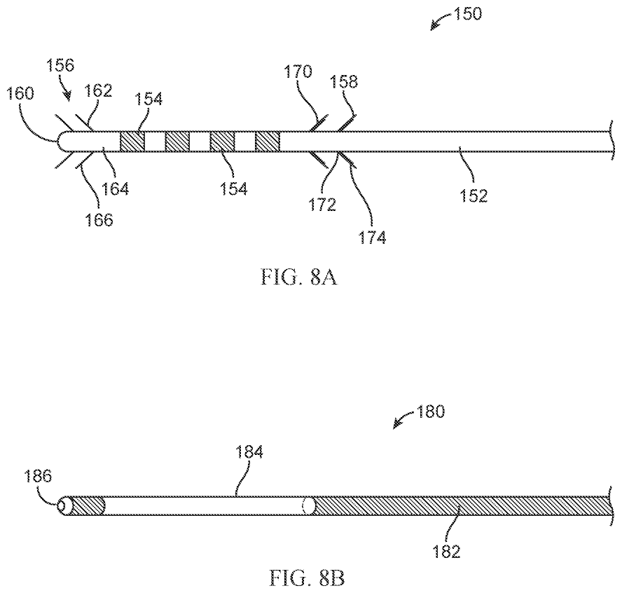

With the above in mind, a portion of one example of a lead assembly 150 useful with the devices, systems, and methods of the present disclosure, for example for percutaneous placement to apply stimulation to a pudendal nerve, is shown in FIG. 8A. The lead assembly 150 includes a cylindrical lead body 152, one or more simulation elements 154, a first fixation unit 156 (referenced generally), and a second fixation unit 158 (referenced generally). The lead body 152 can be of a type and construction conventionally employed with percutaneous stimulation leads, and extends from a distal end 160 to an opposing, proximal end (not shown). The stimulation element(s) 154 can also be of type and construction conventionally employed for delivering stimulation energy to a nerve or other anatomy, and in some embodiments can be a ring electrode, a partial ring electrode, etc. While FIG. 8A illustrates four stimulation elements 154, any other number, either greater or lesser, is equally acceptable. Regardless, the stimulation elements 154 are carried by the lead body 152 and are electrically connected to a respective conductor or wire within a thickness of the lead body 152 as is known in the art. A location of the stimulation elements 154 relative to a length of the lead body 152 can vary, but in general terms are located proximate the distal end 160.

The first fixation unit 156 is carried by or assembled to the lead body 152, and is located proximate the distal end 160. In some embodiments, the first fixation unit 156 is located between a distal-most one of the stimulation elements 154 and the distal end 160. The first fixation unit 156 includes or comprises one or more tines or anchors 162 that are configured to be deflectable from the arrangement of FIG. 8A under the influence of an external force, and to naturally self-assume or self-revert to the shape and/or orientation relative to the lead body 152 of FIG. 8A upon removal of the external force. In some embodiments, the tines 162 of the first fixation unit 156 are configured or formed relative to the lead body 152 to exhibit a distal bias. For example, each of the tines 162 defines a fixed end 164 opposite a free end 166 (labeled for one of the tines 162 in FIG. 8A). The fixed end 164 is attached or fixed to the lead body 152. In the normal state of FIG. 8A, extension of the tine 162 from the lead body 152 includes the free end 166 being radially spaced from the lead body 152, and the free end 166 being distally spaced (relative to a longitudinal direction of the lead body 152) from the fixed end 164. With this orientation or arrangement, following implant of the lead assembly 150 in which the free end 166 is in contact with or embedded within tissue of the patient, the tine 152 will overtly resist movement of the lead body 152 in the distal direction. The tine 162 can be deflected from the arrangement of FIG. 8A (for example during percutaneous delivery), forcing the free end 166 radially inwardly toward the lead body 152; upon removal of this force, the tine 162 will self-revert back to the orientation of FIG. 8A. The first fixation unit 156 can include any number of the tines 162, and the tines 162 can be uniformly or non-uniformly spaced relative to one another about a circumference of the lead body 152.

The second fixation unit 158 is carried by or assembled to the lead body 152, and is located proximal the stimulation element(s) 154. In some embodiments, the second fixation unit 158 is located proximate, but proximally spaced from, a proximal-most one of the stimulation elements 154. The second fixation unit 158 includes or comprises one or more tines or anchors 170 that are configured to be deflectable from the arrangement of FIG. 8A under the influence of an external force, and optionally to naturally self-assume or self-revert to the shape and/or orientation relative to the lead body 152 of FIG. 8A upon removal of the external force. In some embodiments, the tines 170 of the second fixation unit 158 are configured or formed relative to the lead body 152 to exhibit a proximal bias. For example, each of the tines 170 defines a fixed end 172 opposite a free end 174 (labeled for one of the tines 170 in FIG. 8A). The fixed end 172 is attached or fixed to the lead body 152. In the normal state of FIG. 8A, extension of the tine 170 from the lead body 152 includes the free end 174 being radially spaced from the lead body 152, and the free end 174 being proximally spaced (relative to a longitudinal direction of the lead body 152) from the fixed end 172. With this orientation or arrangement, following implant of the lead assembly 150 in which the free end 174 is in contact with or embedded within tissue of the patient, the tine 170 will overtly resist movement of the lead body 152 in the proximal direction. The tine 170 can be deflected from the arrangement of FIG. 8A (for example during percutaneous delivery), forcing the free end 174 radially inwardly toward the lead body 152; upon removal of this force, the tine 170 will self-revert back to the orientation of FIG. 8A. The second fixation unit 158 can include any number of the tines 170, and the tines 170 can be uniformly or non-uniformly spaced relative to one another about a circumference of the lead body 152. While the tines 162, 170 have been described as being configured to self-deploy, in other embodiments one or more of the tines 162, 170 can be configured to achieve the deployed arrangement in response to an operator's action (e.g., insertion or rotation of a steering stylet).

From the above explanations, the first and second fixation units 156, 158 combine to provide the lead assembly 150 with bi-lateral fixation, resisting retrograde and antegrade migration upon final implant. Various tools can be employed to assist with the delivery (e.g., percutaneous delivery) of the lead assembly 150 to a desired target site (e.g., the stimulation element(s) being located in close proximity to a desired segment of a targeted nerve, such as the pudendal nerve). For example, a delivery tool 180 useful with the lead assembly 150 is shown in FIG. 8B. The delivery tool 180 includes a sheath 182 extending from a handle 184. The sheath 182 can assume various forms known in the art, and defines an inner diameter approximating an outer diameter of the lead body 152. Thus, the lead body 152 can be slidably received within the sheath 182, with a hoop strength of the sheath 182 being appropriate to deflect and hold each of the tines 162, 170 against the lead body 152, generating a low delivery profile. Further, the sheath 182 covers the tines 162, 170 during delivery, preventing inadvertent contact between the tines 162, 170 and tissue. Once the lead body 152 has been positioned at a desired location, the sheath 182 is proximally withdrawn, allowing the tines 162, 170 to self-revert to the arrangement of FIG. 8A, engaging with tissue to limit or prevent migration. Notably, the sheath 182 can readily be re-advanced over at least the tines 162 of the first fixation unit to effect re-sheathing when desired.

In some embodiments, the delivery tool 180 can optionally incorporate features that facilitate testing of the stimulation element(s) 154 with the sheath 182 in place over the lead assembly 150. For example, the sheath 182 can include or incorporate one or more windows 184. A size and longitudinal location of the window(s) 184 relative to a distal end 186 of the sheath 182 corresponds with one or more of the stimulation elements 154 relative to the distal end 160. With this construction, when the sheath 182 is arranged over the lead body 152 with the distal end 186 of the sheath 182 proximate or in contact with the distal end 160 of the lead body 152, one or more of the stimulation elements 154 are exposed within or at the window 184 while the tines 162, 170 remain covered by the sheath 182. During an implantation or delivery procedure, the combination lead assembly 150/sheath 182 can be directed to an approximate target site. Once at the approximated location and prior to removal of the sheath 182, an arrangement of the stimulation element(s) 154 relative to targeted anatomy (e.g., a nerve segment) can be tested. For example, stimulation energy can be delivered to the stimulation element(s) 154; because the stimulation element(s) 154 are exposed at the window(s) 184, the exposed stimulation element(s) 154 apply the energy to the patient's anatomy. The clinician can observe the effect(s) of the so-applied stimulation energy and evaluate a location of the stimulation element(s) 154 relative to desired anatomy. As a result of this evaluation, the clinician may decide to reposition the lead body 152 and repeat the testing protocol. Once the clinician is satisfied with the location of the lead body 152 (and in particular the stimulation element(s) 154), the sheath 182 is removed and the implantation procedure completed.

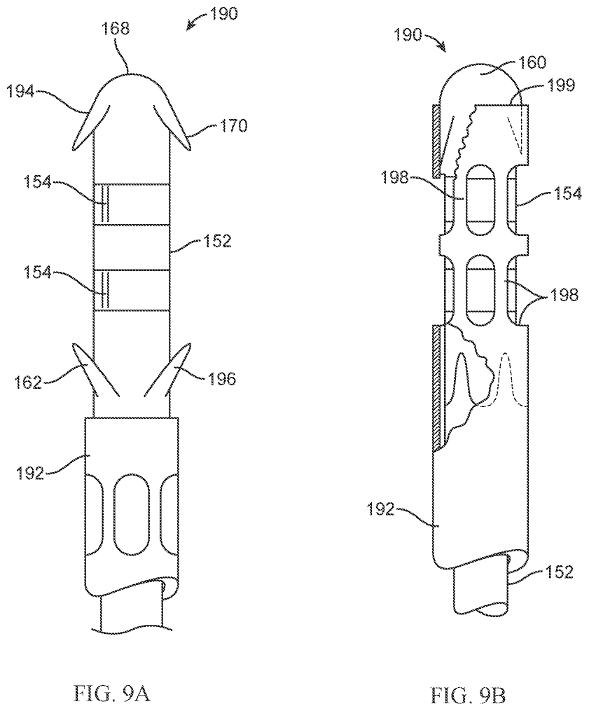

While the lead assembly 150 has been shown and described as providing or including the distally-biased tines 162 proximate the distal end 160 of the lead body 152, and the proximally-biased tines 170 proximal the stimulation elements 154, other constructions are also acceptable. For example, a portion of another example of a lead assembly 190 useful with the devices, systems, and methods of the present disclosure, for example for percutaneous placement to apply stimulation to a pudendal nerve, is shown in FIG. 9A, along with a sheath 192. The lead assembly 190 includes the cylindrical lead body 152 and the stimulation elements 154 as described above, along with a first fixation unit 194 and a second fixation unit 196. The first fixation unit 194 is located proximate the distal end 160 of the lead body 152, and includes one or more of the proximally-biased tines 170. The second fixation unit 196 is located adjacent to, but proximal of, a proximal-most one of the stimulation elements 154, and includes one or more of the distally-biased tines 162.

Where provided, the sheath 192 can assist in the delivery of the lead assembly 190 to a target site. For example, and as shown in FIG. 9B, prior to a delivery procedure, the sheath 192 can be distally advanced over the lead assembly 190, forcing or compressing the tines 162, 170 against the lead body 152 and creating a low delivery profile. The tines 162, 170 remain covered by the sheath 192 during delivery, preventing inadvertent contact between the tines 162, 170 and tissue. Once the lead body 152 has been positioned at a desired location, the sheath 192 is proximally withdrawn, allowing the tines 162, 170 to self-revert to the arrangement of FIG. 9A, engaging with tissue to limit or prevent migration. By providing proximally-biased and distally-biased tines, the lead assembly 190 has bi-lateral fixation.

In some embodiments, the sheath 192 can include or incorporate one or more windows 198. A size and longitudinal location of the window(s) 198 relative to a distal end 199 of the sheath 192 corresponds with one or more of the stimulation elements 154 relative to the distal end 160 of the lead body 152. With this construction, when the sheath 192 is arranged over the lead body 152 with the distal end 199 of the sheath 192 proximate or in contact with the distal end 160 of the lead body 152, the stimulation elements 154 are exposed within or at a corresponding one of the windows 198 while the tines 162, 170 remain covered by the sheath 192. The arrangement of FIG. 9B allows a clinician to perform stimulation testing/lead placement evaluation as described prior to deployment of the tines 162, 170.

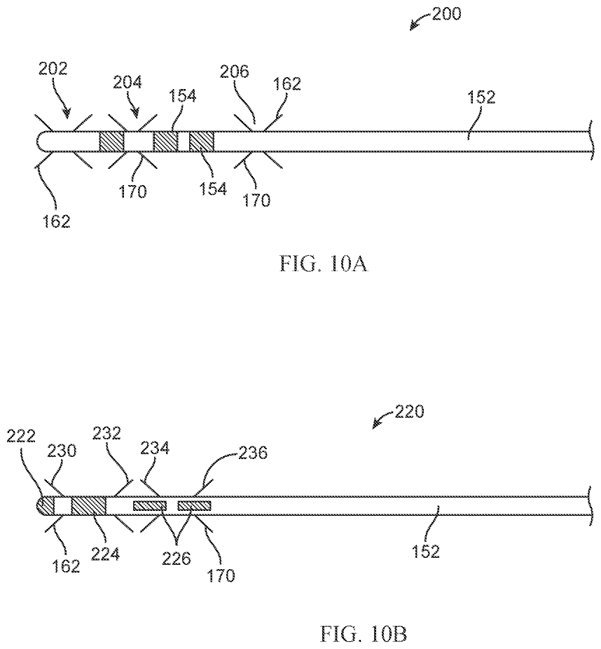

A portion of another example of a lead assembly 200 useful with the devices, systems, and methods of the present disclosure, for example for percutaneous placement to apply stimulation to a pudendal nerve, is shown in FIG. 10A. The lead assembly 200 includes the cylindrical lead body 152 and the simulation elements 154 as described above, along with a first fixation unit 202, a second fixation unit 204, and a third fixation unit 206. The first fixation unit 202 is located along the lead body 152 between the distal end 160 and a distal-most one of the stimulation elements 154. The second fixation unit 204 is located along the lead body 152 between neighboring ones of the stimulation elements 154. The third fixation unit 206 is located along the lead body 152 adjacent, but proximal of, a proximal-most one of the stimulation elements 154. The fixation units 202-206 can be akin to the descriptions above, and include one or more tines or anchors. In some examples, each of the fixation units 202-206 can include a combination of the distally-biased tines 162 and the proximally-biased tines 170. Regardless, the fixation units 202-206 alone or in combination provide the bi-lateral fixation attributes described above.

Various tools can be employed to assist with the delivery (e.g., percutaneous delivery) of the lead assembly 200 to a desired target site (e.g., the stimulation element(s) being located in close proximity to a desired segment of a targeted nerve, such as the pudendal nerve). For example, a delivery tool akin to the delivery tool 180 described above can be employed. The sheath of a delivery tool useful with the lead assembly 200 can form or define two or more spaced apart windows such that when loaded over the lead assembly 200, the stimulation elements 154 are exposed at the windows while the tines 162, 170 of the fixation units 202-206 remain covered.

A portion of another example of a lead assembly 220 useful with the devices, systems, and methods of the present disclosure, for example for percutaneous placement to apply stimulation to a pudendal nerve, is shown in FIG. 10B. The lead assembly 220 includes the cylindrical lead body 152 as described above, various stimulation elements, such as a tip electrode 222, one or more ring electrodes 224, and one or more partial or segmented electrodes 226, a first fixation unit 230, a second fixation unit 232, a third fixation unit 234, and a fourth fixation unit 236. The first fixation unit 230 is located along the lead body 152 proximate the distal end, for example between the tip electrode 222 and neighboring stimulation element, such as the ring electrode 224. The second fixation unit 232 is located along the lead body 152 between neighboring ones of the stimulation elements, such as between the ring electrode and a distal-most one of the segmented electrodes 226. The third and fourth fixation units 234, 236 are located along the lead body 152 so as to be aligned with a corresponding one of the segmented electrodes 226. The fixation units 230-236 can be akin to the descriptions above, and include one or more tines or anchors. In some examples, each of the fixation units 230-236 can include a combination of the distally-biased tines 162 and the proximally-biased tines 170 (several of which are labeled in the view). In other examples, the first and third fixation units 230, 234 can include the distally biased tines 162, and the second and fourth fixation units 232, 236 can include the proximally-biased tines 170. Regardless, the fixation units 230-236 alone or in combination provide the bi-lateral fixation attributes described above.

Various tools can be employed to assist with the delivery (e.g., percutaneous delivery) of the lead assembly 220 to a desired target site (e.g., the stimulation element(s) being located in close proximity to a desired segment of a targeted nerve, such as the pudendal nerve). For example, a delivery tool akin to the delivery tool 180 described above can be employed. The sheath of a delivery tool useful with the lead assembly 220 can form or define two or more spaced apart windows such that when loaded over the lead assembly 220, one or more of the stimulation elements (e.g., one or more of the tip electrode 222, ring electrodes 224, and/or segmented electrodes 226) are exposed at the windows while the tines 162, 170 of the fixation units 230-236 remain covered.

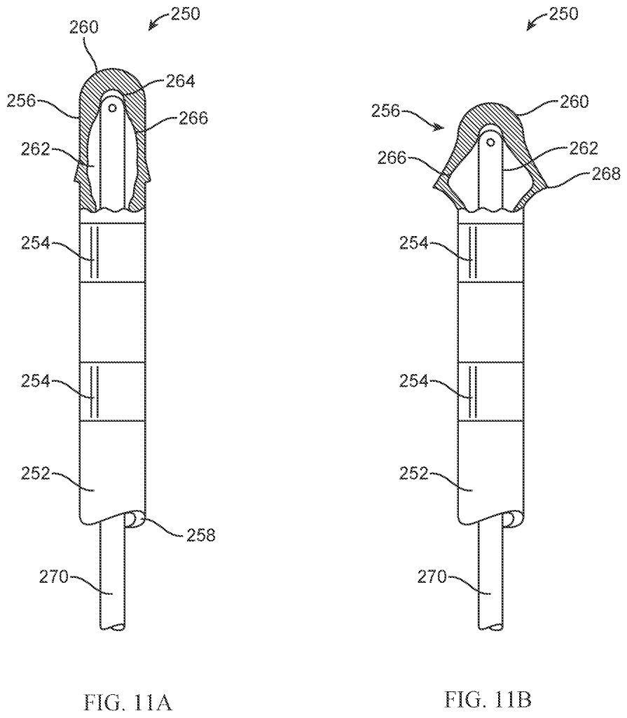

A portion of another example of a lead assembly 250 useful with the devices, systems, and methods of the present disclosure, for example for percutaneous placement to apply stimulation to a pudendal nerve, is shown in FIGS. 11A and 11B. The lead assembly 250 includes a cylindrical lead body 252, one or more stimulation elements 254, and a fixation unit 256. The lead body 252 can assume any of the types known in the art, and defines a lumen 258 (referenced generally). The stimulation element(s) 254 can also be of type and construction conventionally employed for delivering stimulation energy to a nerve or other anatomy, and in some embodiments can be a ring electrode, a partial ring electrode, etc. While FIGS. 11A and 11B illustrates two stimulation elements 254, any other number, either greater or lesser, is equally acceptable. Regardless, the stimulation elements 254 are carried by the lead body 252 and are electrically connected to a respective conductor or wire within a thickness of the lead body 252 as is known in the art.

The fixation unit 256 can include a fixation body 260 formed by or assembled to the lead body 252, This fixation body 260 serves as a distal tip of the lead assembly 250 and defines a chamber 262 terminating at a groove 264. The chamber 262 is open to, or is a continuation of, the lumen 258. The fixation body 260 can be formed of an elastic or similar material configured to self-assume or self-deflect to the predetermined, normal or natural shape in the free state of FIG. 11B. The predetermined shape is characterized by a side or sidewall 266 of the fixation body 260 forming one or more radially-outward projections 268. For example, a radius of the radially-outward projection(s) 268 relative to a centerline of the lead body 252 is greater than an outer radius of the lead body 252. In some embodiments, a radially-outermost extent of the projection(s) 268 can form an edge or corner or other feature conducive to engaging tissue.

As reflected by FIG. 11A, the fixation body 260 is configured to be readily forced to an elongated shape in which the sidewall 266 is relatively straight and the radially-outward projections 268 are minimized or removed or collapsed. Upon removal of the elongation force, the fixation body 260 self-reverts back to the natural shape of FIG. 11B. For example, a stylet or rod 270 can be slidably inserted into the lumen 258, and a distal end 272 thereof located in the groove 264. With further distal advancement of the stylet 270 while the lead assembly 250 is held stationary, or vice-versa, the stylet 270 forces the fixation body 260 to the elongated shape as in FIG. 11A. In this arrangement, the lead assembly 250 has a streamlined shape (e.g., the radially-outward projections 268 do not exists or are minimal) conducive to percutaneous delivery. Once the lead body 250, and in particular the stimulation elements 254, is located at a desired target site (e.g., following testing of the stimulation elements 254), the stylet 270 is removed, allowing the fixation body 260 to revert back to or towards the natural shape. With this transition, the radially-outward projection(s) 268 will engage surrounding tissue, providing fixation of the lead assembly 250 relative to the patient's anatomy at the target site. Under circumstances where re-positioning or removal of the lead assembly 250 is desired, the stylet 270 can be inserted into the lumen 258 and manipulated to force the fixation body 260 to the elongated shape of FIG. 11A; with this arrangement, engagement of the fixation body 260 with surrounding tissue is greatly reduced or eliminated, allowing the lead assembly 250 to easily move relative to the patient's anatomy.

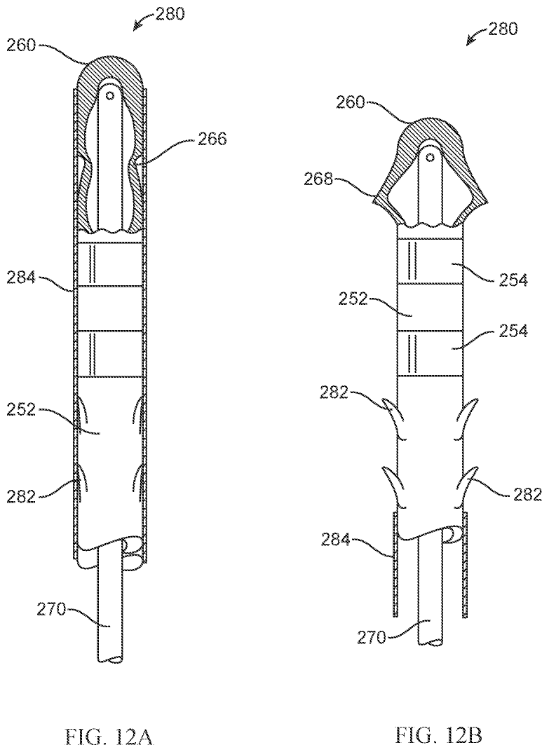

Though not shown, the lead assembly 250 can optionally include additional fixation elements (e.g., tines, anchors, etc.) proximal the stimulation elements 254. With this in mind, a portion of another example of a lead assembly 280 useful with the devices, systems, and methods of the present disclosure is shown in FIGS. 12A and 12B. The lead assembly 280 includes the lead body 252, the stimulation element(s) 254, and the fixation body 260 as described above, along with one more tines or anchors 282. The tine(s) 282 can be located along the lead body 252 adjacent to, but proximal of, the stimulation element(s) 254, and can have any of the forms described above. In some embodiments, one or more or all of the tines 282 can have or exhibit a distal bias as described above, and can be configured to self-revert from a collapsed state to or toward the arrangement of FIG. 12B. In other embodiments, the tines 282 can be configured to achieve the deployed state or shape in response to operator action.

As reflected by FIG. 12A, the fixation body 260 is configured to be readily forced to an elongated shape in which the sidewall 266 is relatively straight and the radially-outward projections 268 are minimized or removed or collapsed as described above. Collapsing of the fixation body 260 can be facilitated by the stylet 270 and/or by a sheath 284 slidably disposed over the lead assembly 280. Regardless, the sheath 284 can further temporarily force the tine(s) 282 to a collapsed state against the lead body 252. In some examples, the lead assembly 280 can be prepared for delivery to a target site by first forcing the fixation body 260 to the elongate shape of FIG. 12A with the stylet 270, and advancing the sheath 284 over the lead assembly 280 to collapse the tines 282. With this arrangement, the lead assembly 280/sheath 284 has a streamlined shape conducive to percutaneous delivery. In this state, the lead assembly 280 can be advanced to an approximate target site. Where desired, a location of the stimulation element(s) 254 relative to desired anatomy (e.g., nerve segment) can be tested, for example by first retracting the sheath 284 proximally beyond the stimulation element(s) (but still over the tines 282) and applying stimulation energy thereto. Once the lead body 252, and in particular the stimulation elements 254, are located at a desired target site, the stylet 270 and the sheath 284 are removed, allowing the fixation body 260 and the tines 282 to revert back to or towards their natural shapes. With this transition, the radially-outward projection(s) 268 and the tines 282 will engage surrounding tissue, providing fixation of the lead assembly 280 relative to the patient's anatomy at the target site. In some embodiments, the fixation body 260 more readily resists retrograde migration and the tines 282 resist antegrade migration, collectively providing the lead assembly with bi-lateral fixation. Under circumstances where re-positioning or removal of the lead assembly 280 is desired, the stylet 270 can be inserted into the lumen 258 and manipulated to force the fixation body 260 to the elongated shape of FIG. 12A; further, the sheath 284 can readily be advanced over the tines 282 (and optionally fixation body 260). With this arrangement, engagement of the fixation body 260 and the tines 282 with surrounding tissue is greatly reduced or eliminated, allowing the lead assembly 280 to easily move relative to the patient's anatomy.

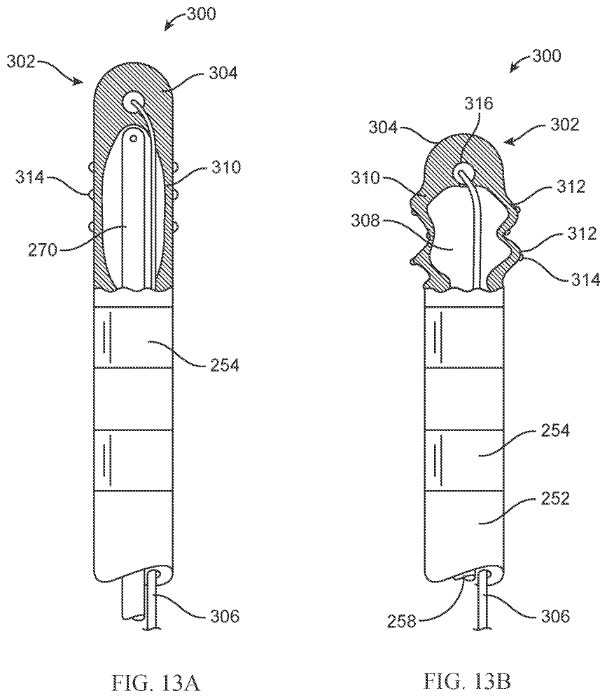

A portion of another example of a lead assembly 300 useful with the devices, systems, and methods of the present disclosure, for example for percutaneous placement to apply stimulation to a pudendal nerve, is shown in FIGS. 13A and 13B. The lead assembly 300 includes the lead body 252, and the stimulation element(s) 254 as described above, along with a fixation unit 302.

The fixation unit 302 includes a fixation body 304 and a line 306. The fixation body 304 defines a chamber 308, and is formed of a material that self-retains a set shape. For example, the fixation body 304 can be forced from the elongated or “straight” shape of FIG. 13A to the deployed shape of FIG. 13B, and optionally will self-retain the so-imparted deployed shape. In some embodiments, the fixation body 304 can be an elastic material configured to self-retain the deployed shape of FIG. 13B. In other embodiments, the deployed shape of FIG. 13B can be pre-set into the fixation body 304, with the fixation body 304 being configured to self-revert to the deployed shape. The deployed shape is characterized by a side or sidewall 310 of the fixation body 304 forming one or more radially-outward projections 312. In other embodiments, the fixation unit 302 is configured such that the line 306 assists in maintaining the fixation body 304 in the deployed shape as described below. In some embodiments, ribs 314 or the like can be formed by or assembled to the fixation body 304 that can one or both of provide enhanced tissue engagement and support of the fixation body 304 in the deployed shape.

The line 306 can be a wire, strand, thread, etc., connected to a thickness of the fixation body 304 distal the chamber 308. For example, the line 306 can be threaded or looped through an aperture 316 in the fixation body 304. In other embodiments, the line 306 can be more permanently affixed to fixation body 304. Regardless, the line 306 extends proximally from the fixation body 304 along the lead body 252 (e.g., the lead body 252 can form a separate lumen for the line 306) to a proximal end thereof. A proximal end of the line 306 can extend proximally beyond the lead body 252 for grasping by a user, or can be connected to an actuator or similar device/mechanism adapted for facilitating the application/removal of a user-applied tensioning force onto the line 306.

As reflected by FIG. 13A, the fixation body 304 is configured to be readily forced to an elongated shape in which the sidewall 310 is relatively straight and the radially-outward projections 312 (FIG. 13B) are minimized or removed or collapsed. For example, the stylet 270 can be inserted into the fixation body 304 and manipulated to “straighten” the fixation body 304 commensurate with the descriptions above for delivery to a target site. Upon removal of the elongation force, the fixation body 304 can be forced to and/or self-reverts back to the deployed shape of FIG. 13B. For example, the line 306 can be pulled or tensioned, forcing the fixation body 304 to the deployed shape. With embodiments in which the fixation body 304 self-retains the deployed shape, the line 306 optionally can then be removed. In other embodiments, the line 306 can be held or locked relative to the lead body 252 under tension, serving to maintain the fixation body 304 in the deployed shape. Regardless, in the deployed shape, the radially-outward projection(s) 312 will engage surrounding tissue, providing fixation of the lead assembly 300 relative to the patient's anatomy at the target site. Under circumstances where re-positioning or removal of the lead assembly 300 is desired, tension in the line 306 (if still present) in removed and the stylet 270 can be inserted into the lumen 258 and manipulated to force the fixation body 306 to the elongated shape of FIG. 13A; with this arrangement, engagement of the fixation body 306 with surrounding tissue is greatly reduced or eliminated, allowing the lead assembly 300 to easily move relative to the patient's anatomy.

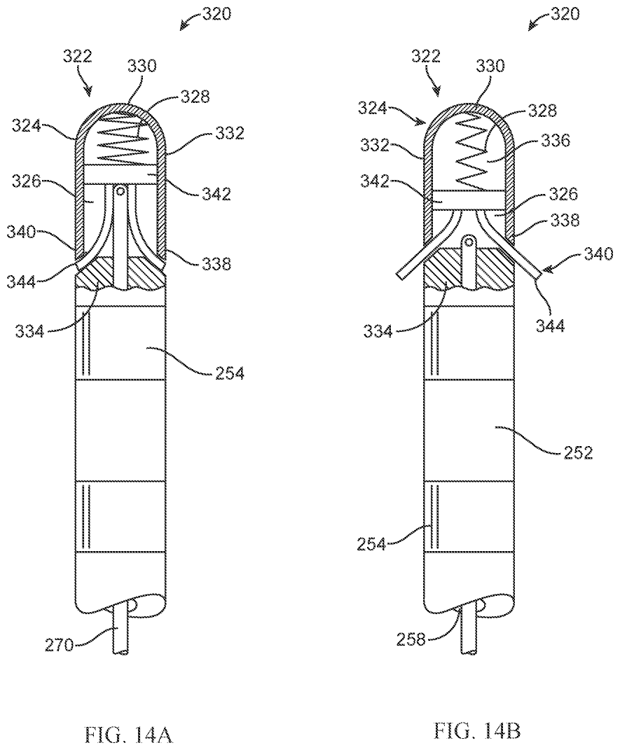

A portion of another example of a lead assembly 320 useful with the devices, systems, and methods of the present disclosure, for example for percutaneous placement to apply stimulation to a pudendal nerve, is shown in FIGS. 14A and 14B. The lead assembly 320 includes the lead body 252, and the stimulation element(s) 254 as described above, along with a fixation unit 322.

The fixation unit 322 includes a housing 324, a tine assembly 326, and a biasing member 328. The housing 324 can be formed by or assembled to the lead body 252, and serves as a distal tip of the lead assembly 320. The housing 324 includes or defines an end wall 330, a sidewall 332, an internal wall 334, and a chamber 336 that is open to the lumen 258. One or more passages 338 are formed through the sidewall 332 proximate the internal wall 334 for reasons made clear below. In some embodiments, a shape of each of the passages 338 has a proximal component in extension from the chamber 336 to an exterior of the housing 324 (e.g., an internal end of the passage 338 at the chamber 336 is distal an opposite, external end at the exterior of the sidewall 332).

The tine assembly 326 can assume a variety of forms, and in some embodiments includes one or more tines 340 and a base 342. The tines 340 each extend from a fixed end attached to or formed by the base 342 to a free end 344 opposite the base 342. The tines 340 can be formed of a rigid yet deformable material appropriate for engaging tissue, such as a mono-filament polymer body. The base 342 is sized and shaped to be slidably disposed within the chamber 336. Upon final construction, the tine assembly 326 is arranged relative to the housing 324 such that base 342 is within the chamber 336, and each of the tines 340 is aligned with a corresponding one of the passages 338.

The biasing member 328 can assume various forms appropriate for applying a force onto the base 342, and in some embodiments is or includes a spring or spring-like body. The biasing member 328 is arranged between and contacts the end wall 330 and the base 342. With this construction, the biasing member 328 applies a force onto the base 342 in the proximal direction (forcing the base 342 away from the end wall 330).

In the normal or deployed state of FIG. 14B, each of the tines 340 project through the corresponding passage 338, with the corresponding free end 344 being located radially outward of the lead body 252. A shape of the passages 338 optionally imparts a proximal bias into each of the tines 340 (e.g., the free end 344 is proximal a point of departure of the tine 340 from the housing 324).

As reflected by FIG. 14A, the fixation unit 322 is configured to be readily forced to a streamlined or delivery state in which the tines 340 are partially or completely retracted within the housing 324. For example, the stylet or rod 270 can be slidably inserted into the lumen 258, and the distal end 272 placed in contact with the base 342. With further distal advancement of the stylet 270 while the lead assembly 320 is held stationary, or vice-versa, a force applied by the stylet 270 onto the base 342 overcomes the force of the biasing member 328, causing the base 342 to move distally. Distal movement of the base 342, in turn, is transferred onto the tines 340, thus retracting the tines 340 into the housing 324. In this arrangement, the fixation unit 322 is streamlined (e.g., the tines 340 minimally project beyond the housing 324, if at all) conducive to percutaneous delivery. Once the lead assembly 320, and in particular the stimulation elements 254, is located at a desired target site (e.g., following testing of the stimulation elements 254), the stylet 270 is removed, allowing the biasing member 328 to force the base 342, and thus the tines 340, to or towards the arrangement of FIG. 14B. In other embodiments, a pull-wire can actively deploy the tines 340 without the need for a passive spring mechanism (e.g., the stylet 270 can push the tine assembly 326 in the opposite direction, retracting the tines 340 for removal or repositioning). In the deployed state, the tines 340 will engage surrounding tissue, providing fixation of the lead assembly 320 relative to the patient's anatomy at the target site. Under circumstances where re-positioning or removal of the lead assembly 320 is desired, the stylet 270 can be inserted into the lumen 258 and manipulated to force the base 342 to the arrangement of FIG. 14A, thus retracting the tines 340; with this arrangement, engagement of the tines 340 with surrounding tissue is greatly reduced or eliminated, allowing the lead assembly 320 to easily move relative to the patient's anatomy.

The fixation units of the present disclosure can assume other forms or formats that may or may not include deployable tines (e.g., the bi-directional tine configurations describe elsewhere). For example, fixation or lead anchoring can be facilitated with circular or helical-shaped barb or sharpened wire capable of being rotated or otherwise articulated to fixate or anchor into targeted anatomy (e.g., muscle, ligament, bone, etc.) in a screw-like manner.

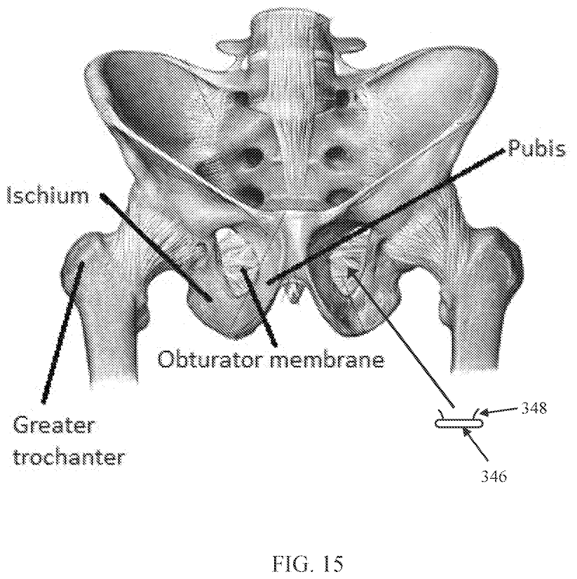

In some embodiments, the systems and methods of the present disclosure can leverage features of naturally occurring anatomy to facilitate anchoring of one or more devices. For example, and as shown in FIG. 15, the obturator foramen is covered by a thick membrane called the obturator membrane. The external and internal obturator muscles cover this membrane, and is considered a safe space anatomically. In some embodiments of the present disclosure, a device 346 useful with the treatment of urinary or bowel incontinence (e.g., a microstimulator, a sensor lead, etc.) can include or carry one or more structures 348, such as deployable tines, configured to pierce through the obturator membrane. Once embedded, the structures fixate or anchor the device at the obturator membrane and at a location useful for incontinence treatment. In other embodiments, the systems and methods of the present disclosure can be configured to anchor a lead (or other device) to the Alcock's canal (FIGS. 4A-4C), for example in locating the stimulation element(s) to apply stimulation energy to the pudendal nerve.

Returning to FIGS. 1-3, the present disclosure includes other formats or techniques for anchoring a device, such as a lead carrying the stimulation element(s) 66, relative to targeted native anatomy. For example, the lead can be connected to a sling-like device carrying engagement structures configured to engage or lock to native ligaments in the pelvic region (e.g., Cooper's ligaments). For example, the engagement structures can be or include tines, sutures, etc. Once deployed, the sling-like device fixates or anchors the corresponding lead relative to the targeted native anatomy. Other anchoring techniques or mechanisms are also acceptable. For example, stimulation leads (and/or IPG) of the present disclosure can be anchored to tissue by direct suturing. With some desired stimulation target sites, the native tissue for direct suturing can be subcutaneous muscle and/or ligaments that are nearby the intended target site.

In some example embodiments, the stimulation element 66 can be provided as part of a cylindrical-type lead configured for placement in the periurethral space. For example, some systems and methods of the present disclosure can include transperineal placement of a cylindrical lead in the periurethral space for direct muscle stimulation of the pelvic floor muscles 18 and/or the external urethral sphincter 34 or other muscles (smooth) of the urethra 14 or bladder 10. Such systems and methods can further operate to stimulate the distal branches or terminal fibers of the pudendal nerve 44. In other, related embodiments, one or more cuff-type electrodes can be delivered via a transperineal approach for placement about or around the distal branches of the pudendal nerve 44. In yet other related embodiments, intramuscular-type electrodes are employed and are delivered via a transperineal approach.

As mentioned above, while a single stimulation element 66 is shown in FIG. 3, in other embodiments, two or three or more of the stimulation elements 66 can be provided, and may or may not have differing formats (or stimulation element components). Thus, one or two or three or more of any of the stimulation arrangements of the present disclosure can be provided with the treatment system for an individual patient. Two or three or more of the various non-limiting examples of stimulation element locations and formats provided in the present disclosure can be combined with a treatment system for an individual patient. For example, some treatment systems of the present disclosure are configured and formatted/programmed to provide stimulation to two or three or more target sites By way of non-limiting example, stimulation can be provided to certain target site(s) intended to treat incontinence when voiding/leakage is not desired, and stimulation to one or more other target site(s) intended to encourage or promote voiding (while at the same time not stimulating target sites otherwise intended to treat incontinence) when voiding is desired. With these and related embodiments, for example, efferent stimulation can be provided to at least one of the multiple target sites, whereas afferent stimulation can be provided to at least another one of the multiple target sites. In yet other embodiments, a single stimulation element 66 can be applied to a nerve that innervates two or more different target muscles or organs; under these circumstances, the systems and methods of the present disclosure can include applying a first stimulation signal format to the nerve to affect a first one of the targeted muscles or organs, and a second stimulation signal format to the nerve to affect a second one of the targeted muscles or organs.

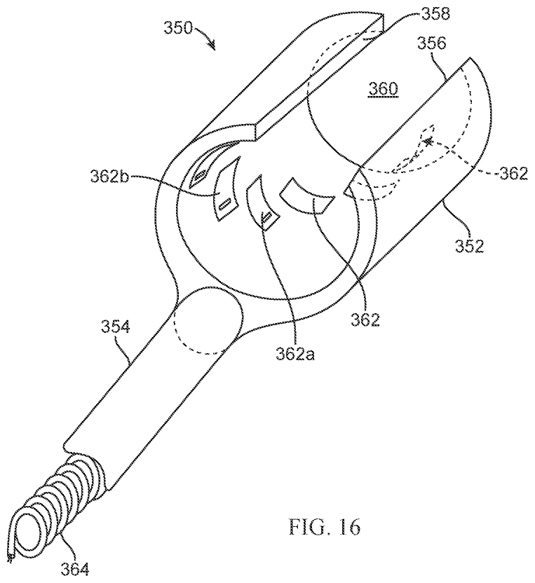

For example, a non-limiting example lead assembly 350 in accordance with principles of the present disclosure is shown in FIG. 16. The lead assembly 350 includes a cuff body 352 and a lead body 354. The cuff body 352 can be of a type or format known in the art, appropriate for placement and self-retention about or over a nerve. For example, the cuff body 352 can form or define opposing edges 356, 358, along with a central passage 360 (referenced generally). The central passage 360 can be accessed via a spacing or gap between the opposing edges 356, 358, with the cuff body 352 configured to normally or naturally self-assume or self-revert to a capture state in which the opposing edges 356, 358 are biased toward one another (it being understood that in the illustration of FIG. 16, the opposing edges 356, 358 have been slightly retracted from one another to better show other features of the lead assembly 350).

The cuff body 352 carries a plurality of stimulating elements or electrodes 362. While six of the electrodes 362 are shown in FIG. 16, any other number, either greater or lesser, is also acceptable. The electrodes 362 can be arranged in an array-like pattern along the cuff body 352. Various ones of the electrodes 362 can be longitudinally and/or circumferentially off-set from one another. For example, the electrode labeled as 362a is off-set from the electrode labeled as 362b in both a longitudinal direction (i.e., in a direction of a longitudinal axis defined by the central passage 360) and a circumferential direction (i.e., relative to a circumference of the shape defined by the cuff body 352). The array reflected by FIG. 16 is but one example of an electrode pattern envisioned by the present disclosure. Electrical connections or wiring 364 for each of the electrodes 362 is carried within the cuff body 352 in an electrically isolated fashion. As is known in the art, the electrode wiring or conductors passes from the base cuff body 352 to the lead body 354 that in turn carries the electrode wiring in an electrically isolated fashion to a proximal region (not shown) formatted for physical and electrical connection to an implantable pulse generator. The wiring or conductors 364 can have a coil format as shown, a straight or cable-like format, etc.

The lead assembly 350 can be useful, for example, in promoting stimulation of specific or selected fibers of a target nerve, for example the pudendal nerve. As a point of reference, the pudendal nerve affects or controls a number of bodily functions or activities, and it can be of value to stimulate only those fiber(s) of the pudendal nerve that will affect the desired bodily function (as opposed to stimulating all fibers of the pudendal nerve). Once implanted over the pudendal nerve, trials or testing can be performed to estimate or determine which fiber(s) each of the electrodes 362 is most likely to affect when energized. The control portion 70 (FIG. 3) can be programmed to then operate the so-identified electrodes for effecting a desired treatment regimen. For example, control portion 70 can be programmed such that the electrode(s) 362 determined to be affect larger motor axons of the pudendal nerve are energized to stimulate or activate the external urethral sphincter, whereas a more continuous stimulation pattern is used to activate smaller afferent fibers for reflex response to mitigate OAB. These and similar field steering or field strength features can be applied with the lead assembly 350, and in particular the array of electrodes 362.

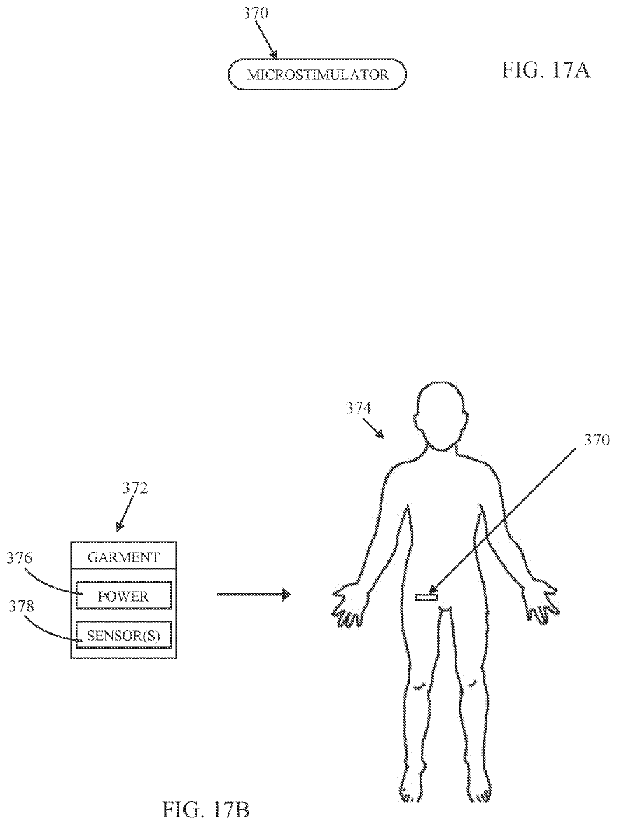

In some examples, the stimulation element(s) of the present disclosure can comprise or be provided with a microstimulator, such as microstimulator 370 schematically represented in FIG. 17A. In some such examples, the microstimulator 370 may comprise at least some of substantially the same features and attributes as the pulse generator 64 (FIG. 3), except being provided in a much smaller sized package for implantation in smaller locations than a full-sized pulse generator would typically be implanted. Moreover, because of its smaller size (relative to a full sized pulse generator), the microstimulator 370 can be implanted in relatively close proximity to the stimulation target, whether or not the microstimulator 370 may have a relatively short lead extending therefrom to carry a stimulation element (and/or sensing element). Via such arrangements, it becomes more likely that the implantable medical elements may be implanted via a single implant-access incision. Moreover, two (or more) of the microstimulators 370 may be implanted at opposite sides of the patient's body to deliver bilateral stimulation.

In sharp contrast, some commercially available pulse generators are of a larger size such that are implanted remotely from the stimulation target and a significantly longer lead extends from the implanted pulse generator to the stimulation target. Among other aspects, some such arrangements may typically involve multiple implant-access incisions.

In some embodiments, the power source of a microstimulator may be re-chargeable via a recharge element positionable external of the patient's body in proximity to the microstimulator to be re-charged. In some non-limiting examples, a wearable garment 372 can be provided, as schematically reflected by FIG. 17B. The garment 372 can assume various forms appropriate for wearing by a patient 374. As a point of reference, FIG. 17B reflects a general position of the implanted microstimulator 370 relative to the patient 374, generally in the pelvic region or lower torso. With this in mind, the garment 372 can be sized and shaped for convenient wearing by the patient 374 over the implant location, such as underwear, shorts, belt, cummerbund, etc. A power element or recharge coil 376 (for recharging the power source of the microstimulator 370) is carried by the garment 372. In particular, a location of the power element 376 along the garment 372 corresponds with the expected manner in which the patient 374 will wear the garment 372 and the expected location of the implanted microstimulator 370. Thus, when the garment is worn by the patient 374 in the expected manner, the power element 376 will naturally align with the implanted microstimulator 370 to facilitate re-charging. Optionally, the garment 372 can carry one or more sensors 378 a location of which also corresponds with the expected manner in which the garment 372 will be worn by the patient 374 and a desired location of the sensor(s) 378 relative to the patient 374 (e.g., where the sensor 378 is intended to sense a parameter indicative of bladder pressure and the garment 372 is, or is akin to, running shorts, the sensor 378 will be located along the garment 372 so that when worn by the patient 372, the sensor 378 is near or over the patient's bladder). The power element 376 and/or the optional sensor(s) 378 can be secured to the garment 372 in various manners. For example, in some embodiments, the power element 376 and/or the optional sensor(s) 378 can be releasably fastened to the garment 372 (e.g., Velcro®), affording a user the ability to remove the electrical components, wash the garment 372, and then re-assemble for later use.

Returning to FIGS. 1-3, some aspects of the present disclosure relate to delivery systems and/or methods for delivering the stimulation element 66 (as provided, for example, as part of a lead, a cuff electrode, a microstimulator, etc.) to an intended target site. In some embodiments, laparoscopic procedures and delivery tools can be employed for the delivery of, for example, any of the leads or cuff electrodes of the present disclosure (e.g., FIGS. 6 and 13A).

In other embodiments, delivery systems of the present disclosure can include various tools, such as a needle, a guide (e.g., akin to a guidewire), and an introducer. With these and related embodiments (for example for the delivery of any of the leads of the present disclosure), the needle tool can include a needle body attached to a handle, with the needle body coated with an electrically insulator material except at the tip. The needle tip is inserted into the patient and advanced to the expected stimulation site. Electrical energy is then delivered through the needle at the tip to stimulate tissue and evaluate whether or not the needle tip is at the desired location. Once it is confirmed that the needle tip is located at the desired target site, a guide is inserted into and through a lumen of the needle and located at the attained target site, and the needle removed. In other embodiments, the handle can be removed from the needle body, such that the needle body can serve as a guide. Regardless, an introducer is inserted over the guide and advanced to locate a distal end of the introducer at the attained target site. The guide is removed, and the lead introduced and advanced to the attained target site through the introducer. In some optional embodiments, the so-positioned lead can be operated to stimulate tissue, allowing the clinician to confirm desired location of the lead before deploying anchor(s) carried by the lead. These techniques can be useful with any of the stimulation target sites of the present disclosure.

In other embodiments, delivery procedures of the present disclosure can include or entail a cut down technique. These and related techniques can be useful, for example, for delivering a cuff electrode of the present disclosure to a main trunk of a targeted nerve.

### Sensors

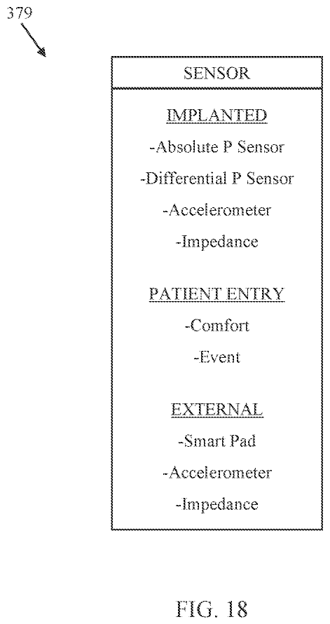

Returning to FIGS. 1-3 and as alluded to above, the IPG 64 can operate (or be prompted to operate) to prompt the delivery of, cease the delivery of, and/or modulate the delivered stimulation signal based upon, or as a function of, one or more sensed parameters of the patient via information generated by the sensor(s) 62. Alternatively or in addition, information from the sensor(s) 62 can be utilized for monitoring the patient and may or may not directly implicate operation of the IPG 64. The sensor(s) 62 can include sensors formatted for implantation into the patient or sensors intended to operate external the patient. While a single sensor 62 is shown in FIG. 3, in other embodiments, two or more of the sensors 62 can be provided, and may or may not have differing formats (or sensor components). Thus, two or more of any of the sensor arrangements of the present disclosure can be provided with the treatment system for an individual patient. Some non-limiting examples of sensors and sensors arrangements useful with the systems of the present disclosure are described in greater detail below. FIG. 18 presents a summary 379 of some of the sensor formats/types that may be employed, including: implanted sensors such as an absolute pressure sensor (e.g., useful for filling and stress event detection), a differential pressure sensor (e.g., useful for filling and event detection), accelerometer (e.g., useful for posture and/or activity driven therapy decisions, event detection, filling detection), impedance (e.g., useful for filling and event detection); patient-entered/self-assessment (e.g., the patient effectively serves as their own sensor by providing feedback via an app or remote device) such as comfort level, event occurrence (e.g., leakage event), and other efficacy-related information (e.g., patient notifies the system when an event occurs to the system can learn/titrate); external sensors such as smart pads, accelerometer, impedance, etc. (it being understood that use of external sensor in place of implanted sensor saves on internal power).

Returning to FIGS. 1-3, in some embodiments, the sensor(s) 62 can assume various forms appropriate for implantation into a human patient (e.g., can include or incorporate a sensor component and an anchoring mechanism or element). In some embodiments the sensor 62 generally includes a sensor component in the form of or akin to a motion-based transducer. In some embodiments, the motion-based transducer sensor component of the sensor 62 can be or include an accelerometer (e.g., a multi-axis accelerometer such as a three-axis accelerometer), a gyroscope, etc., as is known in the art. In some embodiments, the sensor 62 can include or incorporate an accelerometer, which may comprise at least some of substantially the same features and attributes as the sensors described in PCT Publication No. WO 2017/184753 to Dieken et al. and entitled “Accelerometer-Based Sensing for Sleep Disordered Breathing (SDB) Care”, and which is incorporated herein by reference in its entirety. In other embodiments, the sensor 62 can include a differential pressure sensor component as known in the art, a sensor appropriate for sensing EMG information as known in the art, etc. Regardless of an exact form, the sensor component of the sensor 62 is capable of sensing, amongst other things, information indicative of one or more physiological parameters of the patient described below.

A particular format of the sensor 62 can be selected as a function of the parameter(s) to be sensed, and the manner in which the IPG 64 will be controlled in response to the sensed parameter (e.g., the responsiveness or speed of IPG control). The sensor 62 can be a component separate from the stimulation element 66. In other embodiments, the stimulation element 66 can further serve as the sensor from which the parameter of interest can be determined and/or the sensor component of the sensor 62 can be carried by the lead body of the stimulation element 66.

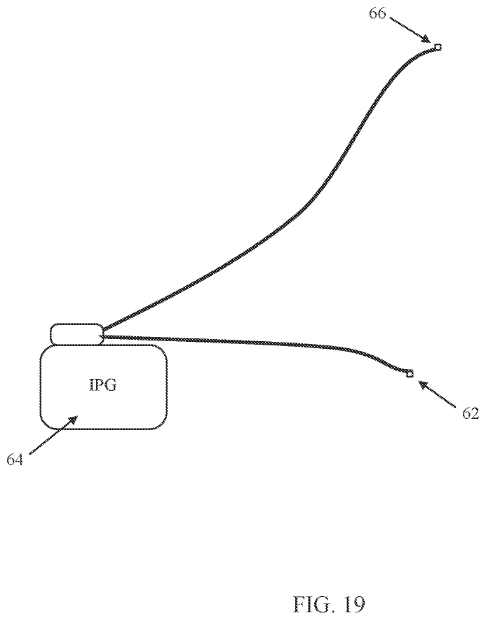

The sensor(s) 62 can be directly connected to the IPG 64 (or the control portion 70 otherwise controlling operation of the IPG 64) via a lead body carrying or terminating at the sensor component as shown, for example, in the simplified representation of FIG. 19. Returning to FIGS. 1-3, in other embodiments, the sensor 62 can be provide as part of a sensing unit that includes or incorporates a computer-like device (e.g., processor, memory and/or computer logic) to process information signaled from the sensor component for delivery to the control portion 70. In yet other embodiments, the sensor 62 can be carried by the IPG 64 assembly (e.g., carried within the housing of the IPG 64 assembly).

In some non-limiting examples, the sensor 62 can be a differential pressure sensor or the like configured to be implanted at a desired target site. For example, with some systems and methods of the present disclosure, the sensor 62 is or includes a differential pressure sensor or the like implanted near the bladder 10 or the urethra 14 to detect pressure increases and/or basal pressure levels of the bladder 10 or the urethra 14. In other optional embodiments, the sensor 62 is or includes a pressure sensor (e.g., a differential pressure sensor) or the like implanted near or on muscles and/or bone in a region of the pelvic floor 18 to sense information indicative of pelvic floor pressure dynamics. With these and related embodiments, the pressure sensor 62 can be placed on the hip bone or the pelvic floor 18.

In some non-limiting examples, the sensor 62 can be a motion-based transducer sensor (e.g., accelerometer such as a three axes accelerometer) configured to be implanted at a desired target site. For example, with some systems and methods of the present disclosure, the sensor 62 is or includes a motion-based transducer sensor component implanted on top of a targeted muscle layer, such as the pelvic floor muscles 18, the detrusor muscle 30, the external urethral sphincter 34, the external anal sphincter, etc. Regardless, in some embodiments, information from a motion-based sensor can be interpreted to derive or predict one or more states of the patient that in turn can be relevant to operation of the treatment system (e.g., prompting delivery of stimulation, ceasing delivery of stimulation, modulating stimulation being delivered, etc.). For example, sensed motion and/or other sensor information can be used to determine or predict a current or future posture of the patient, a current or future activity level of the patient, etc. As described in greater detail below, posture, activity level, and the like (optionally along with other sensor information) can then be reviewed to select a more optimized treatment regimen for the patient.

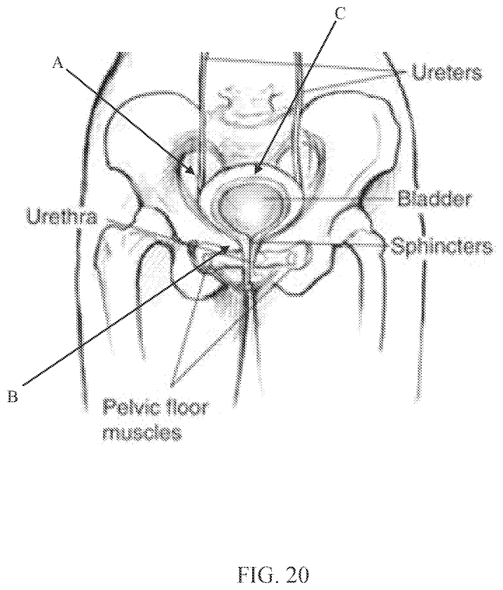

In some non-limiting examples, the sensor 62 can be configured and located to facilitate the sensing of bioimpedance (bioelectrical impedance) information, for example detecting pelvic floor motion that is other indicative of increased pressure (or other circumstances associated with possible leakage or incontinence). As part of the bioimpedance arrangement, the sensor 62 can serve to emit or receive electrical signals appropriate for generating and collecting relevant bioimpedance information. Bioimpedance could be used to sense information indicative of one or more parameters of interest, such as bladder fullness, forces acting on the bladder indicative of a stress incontinence event or normal voiding, body motion, etc. In some embodiments, a large bioimpedance vector is beneficially provided, for example across the bladder 10 (or some portion thereof) of the patient. Other muscles, tissues or organs other than the bladder 10 can also be utilized to provide bioimpedance information of interest. With this in mind, with optional embodiments in which the IPG 64 and the stimulation element 66 are implanted at opposing sides of, for example, the bladder 10, the sensor 62 can be carried within the IPG 64 assembly, with the stimulation element 66 and the sensor 62 being operated to collect bioimpedance information. For example, and with additional reference to FIG. 20, the IPG 64 and the stimulation element(s) 66 are, in some applications, implanted on the same side of the patient's body (e.g., in FIG. 20, a possible implant location of the IPG 64 is indicated generally at A, and a possible implant location of the stimulation element 66 is indicated generally at B). With these and similar implant locations, the impedance vector between the IPG 64 and the stimulation element 66 may cross only part of the bladder, and can be sensitive enough to detect direct changes in volume as well as indirect changes related to the relative positioning of the IPG 64 and the stimulation element(s) 66 as well as other tissues as the bladder volume changes. In related embodiments in which the stimulation element 66 is implanted at a target site along, for example, the bladder 10, the sensor 62 can be provided as part of a sensor lead body electrically coupled to the IPG 64 assembly and arranged to locate the sensor 62 along the bladder 10 generally opposite the stimulation element 66. In yet other embodiments, a bioimpedance signal delivery element (e.g., an electrode) can be provided apart from the stimulation element 66 and that is implanted at a location generally away from a location of the sensor 62. For example, a second impedance sensing lead (carrying one, two or more electrodes) can be inserted from the pocket at which the IPG 64 is implanted to a location (e.g., indicated generally at C in FIG. 20) to improve the measurement vector for impedance and/or allowing the selection of a possible impedance vector between the stimulation element 66, the electrode(s) of the impedance sensing lead, and the IPG 64. In yet other embodiments, the sensor 62 can be configured to be externally worn by the patient, and provided as part of a sensor unit that delivers sensed information wirelessly to the control portion 70.

In some non-limiting embodiments, the sensor 62 can be configured and located to facilitate the sensing of electromyography (EMG) information, for example indicative of motor activity that implicates urinary and/or fecal leakage (or other incontinence-related information). As part of the EMG arrangement, the sensor 62 can serve to emit or receive electrical signals appropriate for generating and collecting relevant EMG information. In some embodiments, EMG information is beneficially provided, for example, relative to the detrusor muscle 30, the external urethral sphincter 34, the external anal sphincter, the abdominal wall, or the pelvic floor muscles 18. Other muscles, tissues or organs can also serve as the target site for EMG information. In some optional embodiments, the stimulation element 66 and the sensor 62 can be implanted and operated to collect EMG information. In other embodiments, an EMG signal delivery element (e.g., an electrode) can be provided apart from the stimulation element 66 and that is implanted at a location along the target muscle generally opposite a location of the sensor 62. In yet other embodiments, a combination of abdominal wall EMG sensor and an accelerometer (or the like) sensor can be provided and acted upon as a surrogate for direct intra-abdominal pressure sensing. The accelerometer signal can generate information implicating occurrence of an acute event by the patient such as coughing, Valsalva, laughing, sneezing, etc., that would cause an increased intra-abdominal pressure without a corresponding abdominal wall contraction (e.g., the abdominal wall EMG sensor may not directly sense the acute event).

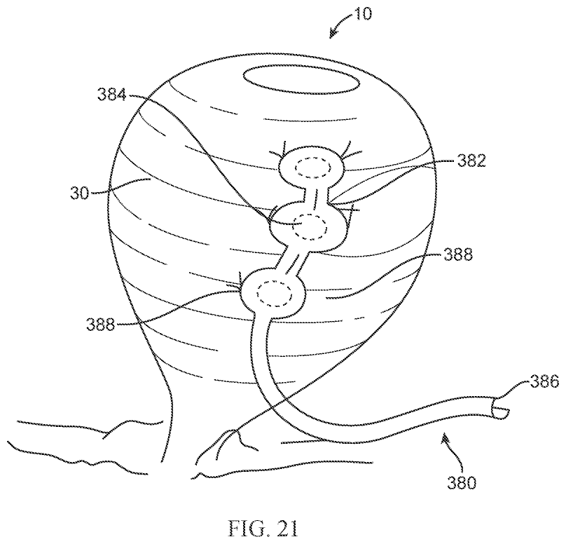

One non-limiting example of an EMG sensor arrangement 380 in accordance with principles of the present disclosure is provided in FIG. 21. The EMG sensor arrangement 380 includes a flexible carrier body 382 maintaining one or more electrodes 384 appropriate for sensing EMG information, and a lead body 386. The electrodes 384 can be formed with or secured to the carrier body 382 in various manners as is known in the art, with corresponding electrical connections or wiring being carried within a thickness of the carrier body 382. The carrier body 382, in turn, is formed of an electrically non-conductive material, and is sized and shaped for attachment to a target site of interest, such as an exterior of the bladder 10 (to thus obtain information relating to the detrusor muscle 30). For example, the carrier body 382 can be relatively thin and flexible, capable of adjusting to a shape and/or contour of the bladder 10. Further, a perimeter shape of the carrier body 382 can provide regions of enlarged surface area appropriate for securement to the bladder 10, for example by sutures 388 that attach the carrier body 382 to a face of the detrusor muscle 30. Regardless, the electrode wiring passes from the carrier body 382 to the lead body 386 that in turn carries the electrode wiring in an electrically isolated fashion to a proximal region (not shown) formatted for physical and electrical connection to an implantable pulse generator or similar electronic device.

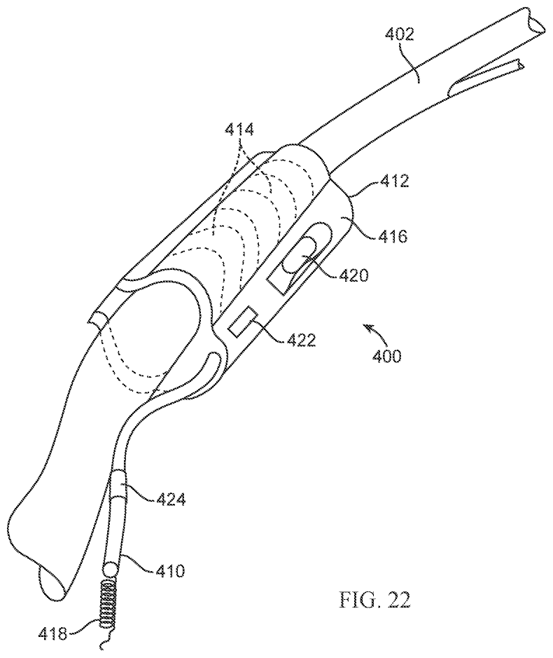

As mentioned above, in some embodiments the sensor(s) can be provided with the lead assembly otherwise carrying the stimulation element(s). For example, FIG. 22 illustrates one non-limiting example of a lead assembly 400 in accordance with principles of the present disclosure having stimulation and sensing features. As a point of reference, FIG. 22 depicts the lead assembly 400 applied to a target nerve 402 (e.g., the pudendal nerve). The lead assembly 400 includes a lead body 410 carrying or attached to a cuff body 412. The cuff body 412 can have a format conducive to atraumatic placement and retention over or about a nerve (e.g., an expandable frame) as is known in the art, and carries one or more stimulating elements or electrodes 414. The stimulating elements 414 are similarly formatted for placement and retention over or about a nerve (e.g., C-shaped or U-shaped electrodes). The cuff body 412 further forms or provides a base 416 in which electrical connections or wiring 418 for each of the stimulating elements 414 is maintained in an electrically isolated fashion. As is known in the art, the electrode wiring passes from the base 416 to the lead body 410 that in turn carries the electrode wiring in an electrically isolated fashion to a proximal region (not shown) formatted for physical and electrical connection to an implantable pulse generator.

The lead assembly 400 further includes one or more sensors each electrically connected to wiring carried by the lead body 410. For example, the lead assembly 400 can include a pressure sensor 420 attached to and carried by the cuff body base 416. The pressure sensor 420 can be of a type known in the art, and in some embodiments is or includes a pressure sensor membrane. The lead assembly 400 can further include one or more bioimpedance sensors or electrodes, for example a first bioimpedance sensor 422 attached to and carried by the base 416, and a second bioimpedance sensor 424 attached to and carried by the lead body 410. A location of a bioimpedance sensor along the lead body 410 (e.g., the second bioimpedance sensor 424) can be selected in accordance with a targeted anatomy upon final implant of the cuff body 412. Alternatively or in addition, one or both of the bioimpedance sensors 422, 424 can be used to sense muscle activity (EMG); the features could be applied to any of the cuff and/or lead assembly constructions of the present disclosure. With these and related embodiments, EMG measurements could be made along a vector from the lead to the pulse generator. In some non-limiting examples, the lead assembly 400 is configured to locate the stimulating elements 414 along the pudendal nerve near the pelvic floor.

Returning to FIGS. 1-3, in some non-limiting embodiments, the sensor 62 can be configured and located to facilitate the sensing of information external the patient. For example, the sensor 62 can be or can be akin to a moisture sensor that is provided as part of a sensor unit configured to be worn by the patient (e.g., a pad) at a location where the sensor 62 detects a leakage event. Other externally-worn sensor arrangements are also envisioned. In some embodiments, information from the external sensor can be communicated to (e.g., via wireless connection), and optionally acted upon, the IMD 60 as described elsewhere in the present disclosure (e.g., where the external sensor 62 is a moisture sensor and senses information indicative of a leakage event, the IMD 60 can be operated (e.g., via a programmed feedback loop) to initiate delivery of stimulation therapy formatted to limit the leakage event from continuing). Additionally or alternatively, information from the external sensor (or any other of the sensor arrangements of the present disclosure) can be communicated to one or both of the patient and caregiver as described below. As a point of reference, knowledge of a leakage event can be useful to a patient, for example, who might not otherwise realize the event is occurring, and/or can be useful to a caregiver in monitoring efficacy of various treatment protocols.

In yet other embodiments, the sensor(s) 62 can be configured and located to sense information indicative of filling or a volume and/or a pressure of the patient's bladder 10 and/or of the patient's rectum 12 (or colon). By way of non-limiting example, information indicate of volume can be obtained by ultrasound, impedance, etc., that is calibrated for the particular patient; similarly, information indicative of pressure can be obtained by a pressure-type sensor located on a waistband of the patient's clothing. Regardless, with these and related embodiments, the IMD 60 can be programmed to operate in response to sensed volume and/or pressure information. For example, where a determination is made that the volume and internal pressure of the patient's bladder (or rectum/colon) is high, it can be assume that the patient is ready to void and the treatment system can be operated so as to encourage voiding (e.g., end stimulation signals intended to relax the detrusor muscle, alert the patient of a need to void, etc.). In yet other embodiments in which a sensor is provided that senses information indicative of the patient's bladder being full, the IMD 60 can be operated, for example, to stimulate baroreceptors of the bladder that in turn signal the patient's brain that the bladder is full (or other afferent stimulation).

In yet other embodiments, information relevant to operation of the treatment system (e.g., prompting the delivery of, ceasing the delivery of, and/or modulating the delivered stimulation signal) can be based upon information entered by the patient at the external device 68. Thus, patient input can serve as, or as part of, the sensor information acted upon by the control portion 70, Leakage events, perceived bladder state, etc., can be entered by the patient and signaled to the control portion 70 in some embodiments.

### Stimulation Methods and Algorithms

Regardless of how the patient-related information is sensed and delivered, some methods of the present disclosure can include prompting the IMD 60 to initiate delivery of, cease delivery of and/or modulate one or more of the stimulation signals (e.g., via programming provided with the control portion 70) based upon or as a function of the sensed patient-related information. In some examples, the stimulation signal is initiated and/or modulated based on sensed patient information. In some examples, one or more of the amplitude, rate, and pulse duration of the stimulation signal is modulated based upon, for example, the sensed patient information. Alternatively or in addition, the duty cycle of the stimulation signal is altered in response to the sensed patient information.

With some example systems and methods of the present disclosure, the stimulation element 66 is located to deliver electrical stimulation sufficient to activate the external urethral sphincter 34 for treatment of urinary incontinence. For example, and as identified in FIG. 2, the stimulation element 66 can be implanted so as to deliver electrical stimulation directly to the external urethral sphincter 34 (designated as location 72 in FIG. 2), with the control portion 70 programmed to prompt the IPG 64 to generate a stimulation signal with parameters (e.g., intensity, frequency, duty cycle, etc.) appropriate to cause the external urethral sphincter 34 to activate or contract. In other embodiments, the stimulation element 66 can be implanted so as to deliver electrical stimulation to the pudendal nerve 44 or an appropriate branch thereof (two possible locations are identified at 74 and 76 in FIG. 2), with the control portion 70 programmed to prompt the IPG 64 to generate a stimulation signal with parameters (e.g., intensity, frequency, duty cycle, etc.) appropriate to cause the external urethral sphincter 34 to activate or contract in response to the pudendal nerve 44. Similar arrangements and configurations are alternatively employed relative to the external anal sphincter for treatment of fecal incontinence. With some embodiments in which the stimulation element 66 is located to stimulate the pudendal nerve 44 (or other nerves) to effect contraction or closure of the external urethral sphincter 34 (or external anal sphincter) for the treatment of stress incontinence, the systems and methods of the present disclosure can be selected to target or stimulate the efferent neurons (as opposed to the afferent neurons). As such, in some embodiments, the systems and methods of the present disclosure can include delivering high frequency stimulation energy, for example on the order of 30 Hz or greater. In other embodiments, the delivered stimulation energy can have a frequency of less than 30 Hz. In yet other embodiments, the delivered stimulation energy can be formatted stimulate afferent neurons.

With some example systems and methods of the present disclosure, the control portion 70 is programmed to prompt the provision of appropriate stimulation energy to the targeted nerve and/or muscle on a fully dynamic basis. For example, the control portion 70 can be programmed to initiate the delivery of stimulation energy upon determining the occurrence of a patient event or circumstance (via information from the sensor 62) indicative of potential leakage (urine leakage or fecal leakage). The control portion 70 can be further programmed to continue the delivery of the stimulation energy for a predetermined length of time and/or until the determined patient event or circumstance has subsided or ended. For example, the control portion 70 can include or be programmed to include or provide a monitoring engine and a therapy engine. The monitoring engine is programmed to evaluate information from the sensor(s) 62 (along with, in some embodiments, information from other sources such as other sensors associated with the patient) to determine or designate the likelihood of a possible leakage event or circumstances (e.g., expansion of the bladder 10). Once the monitoring engine has determined the existence of potential leakage, the therapy engine is prompted to initiate the delivery of stimulation energy with predetermined parameters deemed appropriate to suppress leakage from occurring (e.g., sufficient to activate the external urethral sphincter 34).

With some example systems and methods of the present disclosure, the control portion 70 is programmed to prompt the provision of appropriate stimulation energy to the targeted nerve and/or muscle (including selectively providing simulation to two or three or more target sites) on a dynamic basis and on a basal basis. For example, the control portion 70 can be programmed to provide a basal level of stimulation to help prevent leakage in addition to the dynamic protection against leakage as described above. In some embodiments, the basal stimulation can be modulated to minimize undesired stressing or fatiguing of the muscle(s) being stimulated. For example, the control portion 70 can include or be programmed to include a therapy engine that provides a dynamic mode of operation as described above, and a basal mode of operation. The basal mode can be programmed to modulate the delivered stimulation energy by way of a predetermined on/off duty cycle, ramping up/down the delivered stimulation energy in a predetermined manner, etc. In yet other optional embodiments, the systems and methods of the present disclosure can be configured and programmed to deliver stimulation energy to two (or more) target sites. With these and related embodiments, the basal mode of operation can include rotating between the target site receiving stimulation energy. In yet other embodiments, the basal mode of operation can include increasing the level of delivered stimulation in response to the detection of increasing pressure at one or more locations of the patient (e.g., via information from the sensor 62), such as one or more of the bladder 10, the urethra 14, the pelvic floor 18, the rectum 12, etc. With these and similar embodiments, the basal mode of operation can further include providing a low level or no stimulation energy when the detected pressure is low, and then incrementally increasing the delivered stimulation energy as the detected pressure increases (e.g., akin to functioning of a healthy external urethral sphincter 34). In yet other embodiments, low level stimulation energy (e.g., tonal level) is applied to increase sphincter tone, and dynamic stimulation (or dynamic stimulation mode) with a stimulation higher than or ramped up from the tonal level as described above can be effected when desired or deemed appropriate; with these and related embodiments, the dynamic stimulation would be additive to the tonal level, thereby minimizing the time response to a maximal sphincter contraction. The tonal level or low level stimulation energy can be applied on a constant or continuous basis, or can be applied over intervals of time (e.g., a repeating cycle of 30 seconds on, 10 seconds off) independent of the signals prompting delivery of dynamic or additive stimulation energy.

With some example systems and methods of the present disclosure, the control portion 70 is programmed to prompt the provision of appropriate stimulation energy to the targeted nerve and/or muscle on only the basal basis as described above (e.g., without dynamic support).

With some example systems and methods of the present disclosure, the control portion 70 is programmed to prompt the provision of appropriate stimulation energy to the targeted nerve and/or muscle to treat mixed incontinence. With these and related embodiments, the control portion 70 can be configured or programmed to provide one or both of the dynamic and basal modes of operation as described above to reduce or treat stress incontinence. Further, the control portion 70 can be configured or programmed to prompt the stimulation of a target site(s) appropriate to cause the detrusor muscle 30 to relax (thus preventing or reducing urge incontinence or frequency) in an urge mode of operation. For example, stimulation energy can be applied to stimulate the pelvic floor nerves (sacral, pudendal 44) at levels that cause the detrusor muscle 30 to relax. With these and related embodiments, the therapy engine provided with or programmed to the control portion 70 can include a urge incontinence mode that is programmed to prompt an increase in the delivered stimulation energy (e.g., duty cycle or other stimulation energy parameters) as the bladder 10 is determined to be filling to better ensure appropriate detrusor relaxation.

In optional, related embodiments, the control portion 70 can be programmed to effectuate a progressive recruitment therapy routine. For example, the control portion 70 can be programmed such that upon determining or estimating an initial need to provide incontinence therapy (e.g., onset of a leakage or potential leakage event; sensed pressure at the bladder 10; etc.) to prompt application of stimulation energy to activate the external urethral sphincter 34; as the need for incontinence therapy is determined to have increased over time (e.g., pressure at the bladder 10 is determined to have increased by a predetermined amount over a predetermined period of time and/or exceeds a predetermined threshold), the control portion 70 is programmed to prompt application of additional stimulation energy, for example to further recruit activation of the pelvic floor muscles 18, increased intensity of stimulation energy that activates the external urethral sphincter 34, etc.

The control portion 70 can be programmed to learn over time of circumstances where leakage (or other incontinence event) is likely to occur for the patient, for example by correlating an indicated leakage event from the patient with various sensor information at the time of the leakage event (e.g., information from an activity sensor (e.g., accelerometer), abdominal pressure sensor, bladder volume sensor, EMG sensor(s), patient input, etc.). The learned information can then be applied by the control portion 70 for future stimulation therapy, automatically adjusting or increasing stimulation when sensor information indicates that a leakage event is likely to occur, and at other times avoiding the delivery of unwanted stimulation amplitudes, durations and/or frequency that might otherwise be uncomfortable for the patient. These and other automatic learning algorithms can be utilized to balance control and effect with simple user input regarding comfort and leaks.

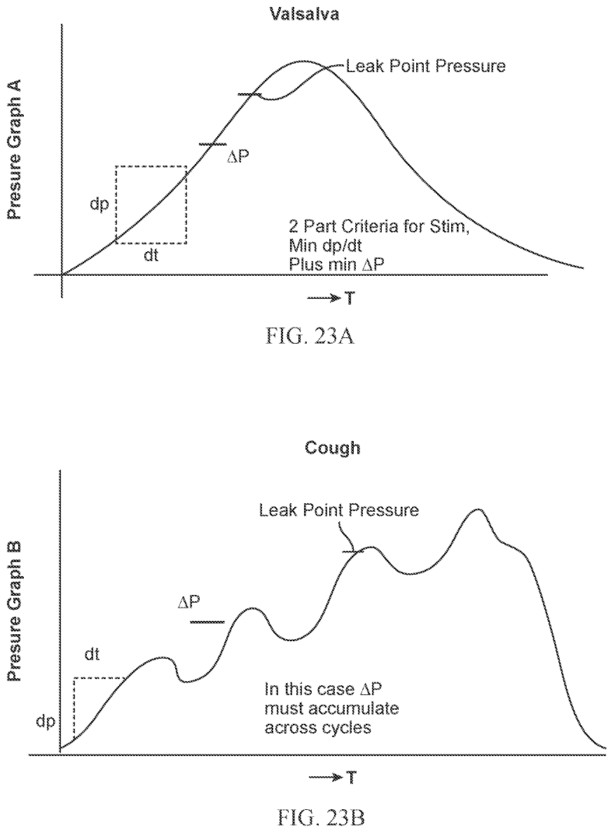

Some non-limiting examples of algorithms useful for predicting occurrence of a possible leakage event based on sensor information are provided in FIGS. 23A and 23B. With this non-limiting embodiment, sensed abdominal pressure can be utilized to predict possible occurrence of a leak event, and action taken (e.g., increased stimulation) prior to the predicted leak event. In the graph of FIG. 23A, abdominal pressure over time is plotted, and a pressure at which a leak event has been determined to likely occur is noted. Where the sensed pressure or change pressure in a single sensing cycle (noted a “ΔP” in FIG. 23A) and the change in pressure over time (dp/dt) is greater than a predetermined or threshold value, stimulation treatment can be initiated or increased. ΔP can effectively be a duration for which the dp/dt occurs so that it is not too short. dp/dt and ΔP can be patient configurable or adjusted automatically based, for example, on a feedback algorithm from the patient (e.g., number of voids, volume/pad weight, etc.) and/or an external sensor(s). Alternatively or in addition, with the algorithm implicated by the plot of FIG. 23B, initiation or increase in stimulation treatment occurs when the sensed change in pressure threshold is determined to have occurred over a predetermined number of sensing cycles (thereby accounting for occurrence of a temporary increase in abdominal pressure that might not directly implicate an expected leak event based on a pressure, such as when the patient coughs). With the examples of FIG. 23B, ramping up and ramping down in the applied stimulation could be used; both would be optional because it could delay maximal therapeutic effect when ramping up and increase overall stimulation time when ramping down. In other embodiments, with any of the algorithms implicated by FIGS. 23A and 23B, negative changes in pressure can be detected via the same mechanism (dp/dt and time) and used to inhibit stimulation or end stimulation.

Returning to FIGS. 1-3, the stimulation therapy algorithms or protocols of the present disclosure (as implemented, for example, by the control portion 70) can, in some non-limiting examples, provide for dynamic stimulation level adjustment or modulation based, for example, on information from the sensor(s) 62 under one or more scenarios. In some embodiments, the control portion 70 is programmed to increase stimulation delivered to the patient in response to an elevated change in a sensed or monitored parameter of the patient, for example intra-abdominal pressure (e.g., the sensor 62 is sensing a property of the patient that indicates abdominal pressure or intra-abdominal pressure, and the control portion 70 determines and monitors the change in the pressure over time (dp/dt)). If, for example, the determined dp/dt is high (e.g., exceeds a predetermined threshold value), stimulation intensity is automatically increased (proportionally, in a step level, or some other relationship). This could address, for example, more severe events experienced by the patient, such as a more aggressive cough with higher level muscle contraction. The elevated stimulation intensity might be uncomfortable, but can be selected to be acceptable to the patient.

In some embodiments, the control portion 70 is programmed to increase stimulation delivered to the patient in response to an elevated change in a sensed or monitored parameter of the patient, for example intra-abdominal pressure (e.g., the sensor 62 is sensing a property of the patient that indicates abdominal pressure or intra-abdominal pressure, and the control portion 70 determines and monitors the change in the pressure over time (dp/dt)). If, for example, monitored pressure is high but not at a level implicating the delivery of stimulation (e.g., a current pressure does not exceed a predetermined threshold) and a subsequent rapid or faster increase in dp/dt (e.g., dp/dt exceeds a predetermined threshold value) to a pressure level that does implicate the delivery of stimulation, the stimulation intensity prompted by the control portion 70 would be higher than a normal or nominal intensity.

In some embodiments, the control portion 70 is programmed to increase stimulation delivered to the patient in response to an elevated change in a sensed or monitored pressure parameter (dp/dt) of the patient as a function of sensed volume (e.g., bladder volume). If, for example, the monitored change in pressure (dp/dt) implicates the delivery of stimulation and the monitored volume is high (e.g., exceeds a threshold value), the stimulation intensity prompted by the control portion would be higher than a normal or nominal intensity.

In some embodiments, the control portion 70 is programmed to decrease or suppress stimulation as a function of sensed volume (e.g., bladder volume). For example, the control portion 70 can be programmed to suppress or not deliver stimulation if the monitored volume is below a threshold value. Alternatively or in addition, the control portion 70 can be programmed to suppress or not deliver stimulation if the monitored volume is deemed to be low (e.g., below a threshold) unless the monitored pressure (e.g., intra-abdominal pressure) and/or change in a sensed or monitored pressure parameter (dp/dt) of the patient is high (e.g., exceeds a threshold).

In some embodiments, the control portion 70 is programmed to provide an increased stimulation intensity (as compared to a normal or nominal stimulation intensity being delivered to the patient, or to be delivered under a dynamic mode of operation) as a function of a body position of the patient. For example, the control portion 70 can be in communication with a sensor(s) providing information indicative of the patient's body position (e.g., an accelerometer). Where the body position information indicates that the patient is standing, for example, the control portion 70 is programmed to prompt delivery of an increased stimulation intensity. Alternatively or in addition, in some embodiments the control portion 70 is programmed to implement an increased sensitivity factor responsive to dp/dt based upon a sensed body position of the patient. For example, where the body position information indicates that the patient is prone or flexing at the torso (and thus naturally causing an increase in the patient's intra-abdominal pressure), the control portion 70 can be programmed to increase a sensitivity to dp/dt information and/or to increase delivered stimulation intensity above the normal or nominal dynamic level.

In some embodiments, the control portion 70 is programmed to provide an increased stimulation intensity (as compared to a normal or nominal stimulation intensity being delivered to the patient, or to be delivered under a dynamic mode of operation) as a function of sensed movement of the patient. Where the body movement information indicates that the patient is running or jumping, for example, the control portion 70 is programmed to prompt delivery of an increased stimulation intensity.

With these and related embodiments (in which the control portion 70 receives and is programmed to act upon information indicative of one or more of activity level, body position, and/or pressure), the control portion 70 can be programmed with an algorithm that considers a monitored activity level of the patient (e.g., sensor information indicative of activity level) and adjusts the applied background stimulation. Further, the algorithm operates to allow voiding/disable stimulation when it is determined that the patient is sedentary and a slow/steady pressure increase is occurring, for example. In related embodiments, the algorithm(s) can be programmed to distinguish a slow/steady pressure increase from a rapid pressure increase; although the patient may be determined to be sedentary, a rapid pressure increase may not implicate a desire to void (e.g., a sneeze or other event causing a rapid pressure increase can be interpreted as meaning that an otherwise sedentary patient does not desire to void, and thus stimulation will not be disabled). These and other algorithms of the present disclosure may also consider posture, examples of which are provided below.

In some embodiments, the control portion 70 is programmed such that a sensitivity factor (or likelihood of delivering stimulation) implemented by the control portion 70 in determining whether or not to prompt delivery of stimulation can be adjusted by the patient. For example, the patient can decrease the stimulation factor (and thus decrease the likelihood of stimulation being delivered) when more comfort is desired and the patient less concerned with a possible minor leak event (e.g., the patient is at home). Conversely, the patient can increase the stimulation factor (and thus increase the likelihood of stimulation being delivered) when in a situation where a possible leak event is less acceptable (e.g., the patient is at a gym or other public setting) and more stimulation sensation is an acceptable tradeoff.

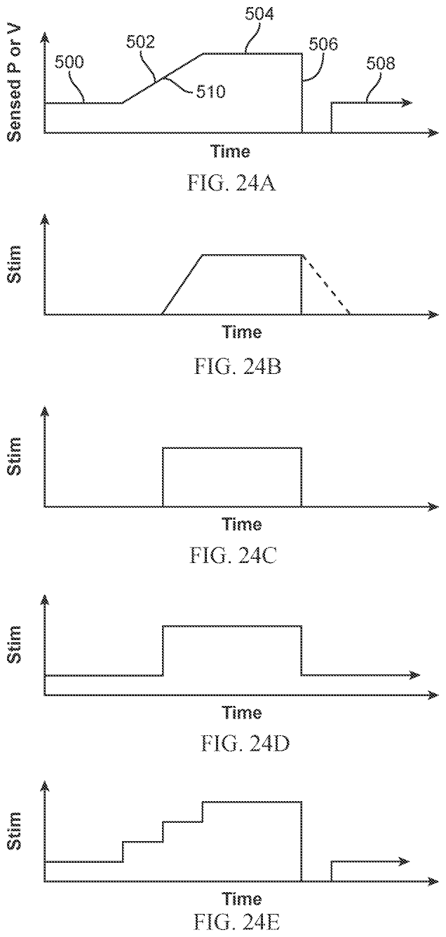

Non-limiting examples of possible stimulation protocols or modulations implemented by the systems and methods of the present disclosure in accordance with the descriptions and algorithms above are provided in FIGS. 24A-24E. The plot of FIG. 24A represents a monitored or sensed physical parameter of the patient relating to continence, for example pressure P (e.g., bladder pressure, abdominal pressure, etc.), volume V (e.g., bladder volume), etc., over time. The monitored parameter is relatively constant over a baseline or normal period 500, gradually increases over an increasing period 502 (e.g., indicative of increased urine in the bladder) until reaching a heighted level where the monitored parameter remains relatively constant over a heightened period 504, and then drops to zero or near zero at 506 (e.g., indicative of the patient voiding). The monitored parameter later rises to the normal level at 508. In some embodiments of the present disclosure, sensed information implicating the monitored parameter or trace of FIG. 24A is provided to or acted upon the control portion 70 (FIG. 3). In related embodiments, the control portion 70 can be programmed to designate or respond to a threshold value or level in the monitored parameter. For example, one possible threshold 510 is identified in FIG. 24A that is attained or occurs along the increasing period 502; the threshold 510 could represent an absolute value of the monitored parameter, the change in the monitored parameter over time, etc.

FIG. 24B represents one stimulation protocol or algorithm implemented by some embodiments of the present disclosure responsive to the monitored parameter of FIG. 24A. With the approach of FIG. 24B, stimulation (“Stim”) is initiated when the threshold 510 is reached (e.g., when the monitored parameter is at the normal level (or otherwise below the threshold 510), no stimulation energy is delivered to the patient). Once the monitored parameter reaches the threshold 510, stimulation energy begins and gradually increases (e.g., intensity, pulse width, frequency, etc.), optionally in a predetermined manner. The delivered stimulation continues to increase until the monitored parameter is identified as having transitioned to the heighted period 504 (i.e., the monitored parameter can be viewed as being elevated, but no longer increasing); at this point, the stimulation energy continues to be delivered to the patient, but at a more constant level. Finally, when the monitored parameter drops below the threshold (e.g., the drop at 506), stimulation energy is stopped or no longer delivered to the patient, or can be ramped down (indicated by a dotted line in FIG. 24B) in other embodiments.

FIG. 24C represents another stimulation protocol or algorithm implemented by some embodiments of the present disclosure responsive to the monitored parameter of FIG. 24A. With the approach of FIG. 24C, stimulation (“Stim”) is initiated when the threshold 510 is reached (e.g., when the monitored parameter is at the normal level (or otherwise below the threshold 510), no stimulation energy is delivered to the patient). Once the monitored parameter reaches the threshold 510, stimulation energy is delivered at a set intensity level and remains at this set level until the monitored parameter drops below the threshold (e.g., the drop at 506). At this point, stimulation energy is stopped or no longer delivered to the patient.

FIG. 24D represents another stimulation protocol or algorithm implemented by some embodiments of the present disclosure responsive to the monitored parameter of FIG. 24A. With the approach of FIG. 24D, basal or low level stimulation (“Stim”) is continuously delivered to the patient regardless of whether the monitored parameter has reached or attained the threshold 510, and thus occurs during the normal period 500. Once the monitored parameter reaches the threshold 510, an intensity of the stimulation energy being delivered is increased to a second level and remains at this second level until the monitored parameter drops below the threshold (e.g., the drop at 506). At this point, the stimulation energy intensity is reduced to the basal or low level.

FIG. 24E represents another stimulation protocol or algorithm implemented by some embodiments of the present disclosure responsive to the monitored parameter of FIG. 24A. With the approach of FIG. 24E, basal or low level stimulation (“Stim”) is continuously delivered to the patient regardless of whether the monitored parameter has reached or attained the threshold 510, and thus occurs during the normal period 500. Once the monitored parameter is identified as increasing (i.e., initiation of the increasing period 502), an intensity of the stimulation energy being delivered is increased. The increase in stimulation energy intensity is formatted to occur in a stepwise fashion as implicated by FIG. 24E. So long as the monitored parameter is identified as increasing or at least not decreasing faster than a specific rate or dropping below an established value, an intensity of the stimulation energy being delivered to the patient is periodically increased. The delivered stimulation continues to increase in a step-like manner until the monitored parameter is identified as having transitioned to the heighted period 504 (i.e., the monitored parameter can be viewed as being elevated, but no longer increasing); at this point, the stimulation energy continues to be delivered to the patient, but at a more constant level. Finally, when the monitored parameter drops in a rapid manner (e.g., the drop at 506), stimulation energy is stopped or no longer delivered to the patient. The basal or low level stimulation is reinitiated once the monitored parameter is deemed as being at the normal level (i.e., 508 in FIG. 24A).

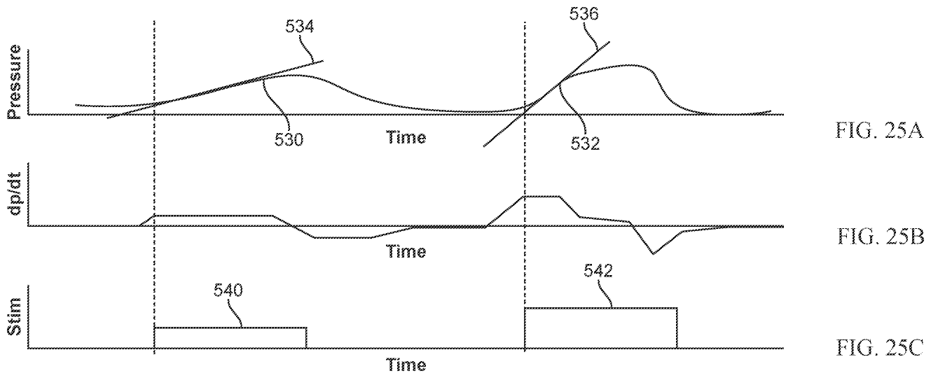

Non-limiting examples of possible stimulation patterns or protocols implemented by the systems and methods of the present disclosure in accordance with the descriptions and algorithms above are provided in FIGS. 25A-25C. The plot of FIG. 25A represents a monitored or sensed physical parameter of the patient relating to continence, for example pressure (e.g., bladder pressure, abdominal pressure, etc.), volume (e.g., bladder volume), etc., over time. The monitored parameter is shown to exhibit a first increasing period 530 and a second increasing period 532. An instantaneous change in the monitored parameter over time (e.g., instantaneous change in intra-abdominal pressure over time or dp/dt) can be determined by a slope of the plot line, and is indicated at 534 for the first increasing period 530, and at 536 for the second increasing period 532. The plot of FIG. 25B reflects the instantaneous change in the monitored parameter (e.g., dp/dt) over time. Slope and absolute pressure change information can also be employed.

FIG. 25C represents one stimulation protocol or algorithm implemented by some embodiments of the present disclosure responsive to the monitored parameter of FIGS. 25A and 25B. With the approach of FIG. 25C, stimulation level (“Stim”) is increased (e.g., voltage, current, pulse width, frequency, etc.) when the instantaneous change in the monitored parameter (e.g., dp/dt) is high (e.g., exceeds a predetermined threshold), and continues at the increased level until the instantaneous change is reduced (e.g., falls below a predetermined threshold). The increase in stimulation could be increased proportionally, in a step level, or some other relationship. For example, the instantaneous change in the monitored parameter associated with the first increasing period 530 results in a first stimulation level 540, whereas the higher instantaneous change in the monitored parameter associated with the second increasing period 532 results in a second, higher stimulation level 542. These optional embodiments could address more severe events experienced by the patient, such as a more aggressive cough with higher level muscle contraction.

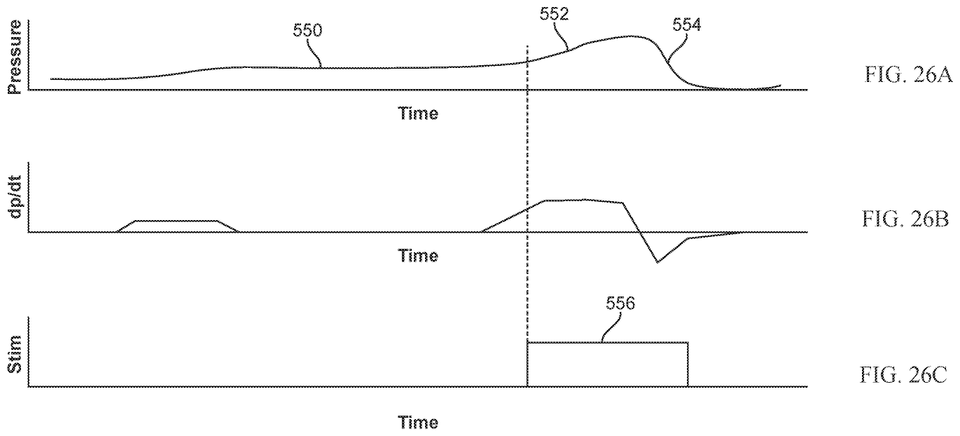

Non-limiting examples of possible stimulation patterns or protocols implemented by the systems and methods of the present disclosure in accordance with the descriptions and algorithms above are provided in FIGS. 26A-26C. The plot of FIG. 26A represents a monitored or sensed physical parameter of the patient relating to continence, for example pressure (e.g., bladder pressure, abdominal pressure, etc.), volume (e.g., bladder volume), etc., over time. The monitored parameter is shown to be relatively steady over a first period 550 and then experiences an increase 552 followed by a decrease 554. The plot of FIG. 26B reflects the instantaneous change in the monitored parameter (e.g., dp/dt) over time as described above.

FIG. 26C represents one stimulation protocol or algorithm implemented by some embodiments of the present disclosure responsive to the monitored parameter of FIGS. 26A and 26B. With the approach of FIG. 26C, stimulation level (“Stim”) is increased (e.g., voltage, current, pulse width, frequency, etc.) when the instantaneous change in the monitored parameter (e.g., dp/dt) increases (e.g., at 552 of FIG. 26A), stimulation delivery, or higher level of stimulation than normal, is triggered at 556 (e.g., dp/dt exceeds a threshold value). Thus, for example, if pressure (or other monitored parameter) is high but not enough to trigger stimulation, then a subsequent detection of an instantaneous increase in a monitored parameter (e.g., dp/dt) would result in delivery of stimulation, where this same level of instantaneous increase would not result in delivery of stimulation if the pressure (or other monitored parameter) were below an established value. Similar, fast increase (e.g., greater dp/dt) with an elevated pressure (or other monitored parameter) would trigger a stimulation output of a higher level than normal.

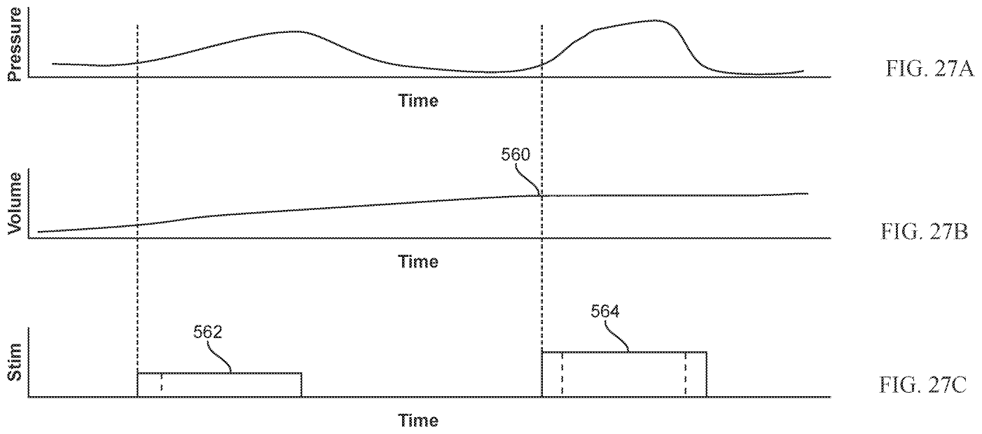

Non-limiting examples of possible stimulation patterns or protocols implemented by the systems and methods of the present disclosure in accordance with the descriptions and algorithms above are provided in FIGS. 27A-27C. The plot of FIG. 27A represents a monitored or sensed pressure of the patient (e.g., bladder pressure, abdominal pressure, etc.). By way of reference, the plot of FIG. 27A is the same as FIG. 25A; thus, an instantaneous change in pressure (dp/dt) implicated by the graph of FIG. 27A is the same as the instantaneous change in pressure (dp/dt) reflected by FIG. 25B. The plot of FIG. 27B represents a monitored or sensed volume (e.g., bladder volume) of the patient over time, contemporaneous with the monitored pressure. The monitored volume is shown as gradually increasing to and beyond a threshold level 560.

FIG. 27C represents one stimulation protocol or algorithm implemented by some embodiments of the present disclosure responsive to the monitored parameters of FIGS. 27A and 27B. With the approach of FIG. 27C, stimulation level (“Stim”) can be delivered at a normal level in response to, for example, the instantaneous change in the monitored pressure (e.g., dp/dt) exceeding a predetermined threshold as indicated, for example, at 562. An alternative starting point for delivering the stimulation 562 is shown with dashed lines in FIG. 27C. Under circumstances where the instantaneous change in pressure implicates the delivery of stimulation and the monitored volume is high (e.g., exceeds the threshold 560), then the stimulation level or intensity (e.g. voltage, current, pulse width, frequency, etc.) delivered to the patient is increased as compared to the normal level (as at 564). Thus, for example, if bladder volume is high, then stimulation levels would be higher for a level of dp/dt as compared to normal bladder levels. Alternative start/stop points for the stimulation 564 is shown with dashed lines in FIG. 27C.

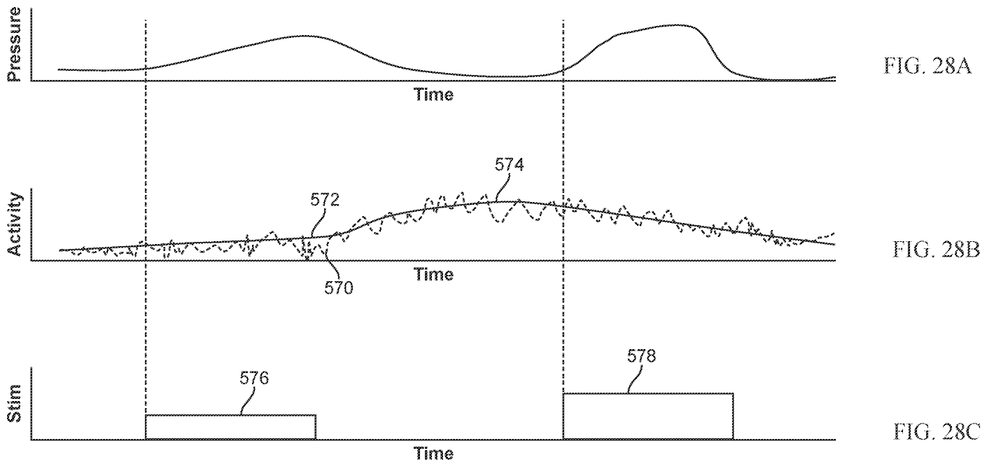

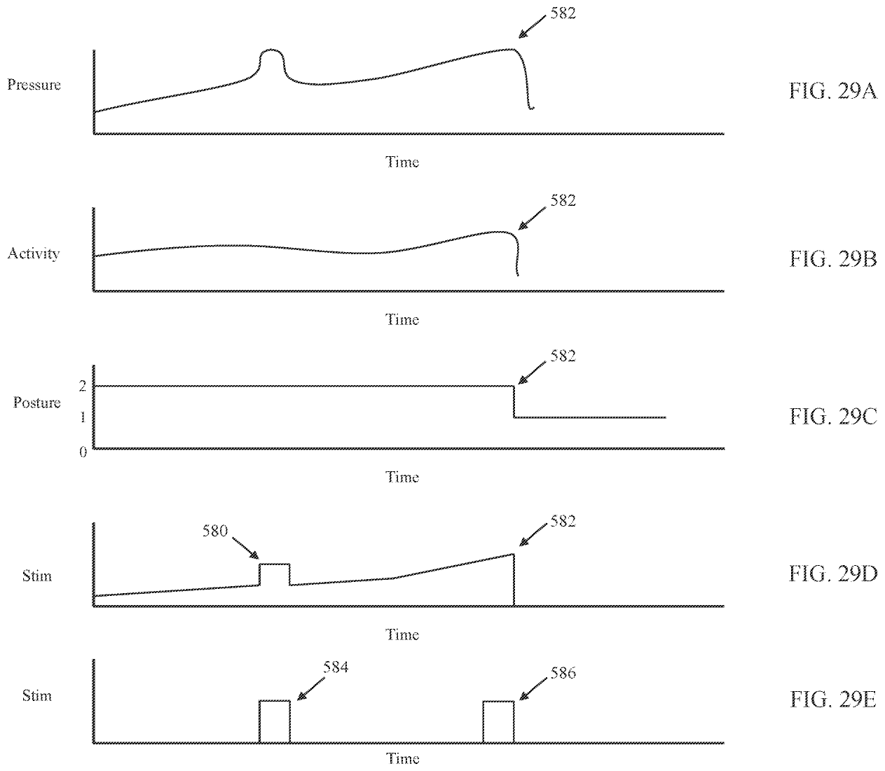

Non-limiting examples of possible stimulation patterns or protocols implemented by the systems and methods of the present disclosure in accordance with the descriptions and algorithms above are provided in FIGS. 28A-28C. The plot of FIG. 28A represents a monitored or sensed pressure of the patient (e.g., bladder pressure, abdominal pressure, etc.). By way of reference, the plot of FIG. 28A is the same as FIG. 25A; thus, an instantaneous change in pressure (dp/dt) implicated by the graph of FIG. 28A is the same as the instantaneous change in pressure (dp/dt) reflected by FIG. 25B. The plot of FIG. 28B represents monitored or sensed activity of the patient over time, contemporaneous with the monitored pressure. The monitored activity can, for example, be generated or implicated by an accelerometer or the like as an acceleration level, mean or RMS (e.g., the jagged line 570 in FIG. 28B represents an actual acceleration signal generated by individual actions such as jumping, running, etc.; the solid line 572 in FIG. 28B represents a running average or RMS value of the acceleration that can be utilized for the algorithm to use to decide if activity levels will increase stimulation). The monitored activity level is shown as exhibiting an elevated period 574.

FIG. 28C represents one stimulation protocol or algorithm implemented by some embodiments of the present disclosure responsive to the monitored parameters of FIGS. 28A and 28B. With the approach of FIG. 28C, stimulation level (“Stim”) can be delivered at a normal level in response to, for example, the instantaneous change in the monitored pressure (e.g., dp/dt) exceeding a predetermined threshold as indicated, for example, at 576. Under circumstances where the instantaneous change in pressure implicates the delivery of stimulation and the monitored activity is high (e.g., exceeds a threshold as in the elevated period 574), then the stimulation level or intensity (e.g. voltage, current, pulse width, frequency, etc.) delivered to the patient is increased as compared to the normal level (as at 578). Thus, for example, dynamic stimulation levels can be increased over nominal if the patient's body movement is unfavorable, such as running or jumping.

Non-limiting examples of possible stimulation patterns or protocols implemented by the systems and methods of the present disclosure in accordance with the descriptions and algorithms above are provided in FIGS. 29A-29E. The plot of FIG. 29A represents a monitored or sensed pressure of the patient (e.g., bladder pressure, abdominal pressure, etc.). The plot of FIG. 29B represents monitored or sensed activity of the patient over time, contemporaneous with the monitored pressure. The monitored activity can, for example, be generated or implicated by an accelerometer or the like. The plot of FIG. 29C represents a monitored or sensed posture of the patient over time, contemporaneous with the monitored pressure. The monitored posture can, for example, be generated or implicated by information from an accelerometer or the like and based upon which a determination can be made as to a likely posture of the patient (e.g., in the plot of FIG. 29C, the designation “2” is assigned to a determination that the patient is likely upright or standing, the designation “1” is assigned to a determination that the patient is likely sitting, and the designation “0” is assigned to a determination that the patient is likely prone or lying down).

FIG. 29D represents one stimulation protocol or algorithm implemented by some embodiments of the present disclosure responsive to the monitored parameters of FIGS. 29A-29C. With the approach of FIG. 29D, while the patient is fairly active and upright, stimulation level (“Stim”) can be delivered at a normal level in response to, for example, the measure pressure or instantaneous change in the monitored pressure (e.g., dp/dt), with the stimulation slightly increasing with gradually increasing pressure. Under circumstances where a pressure spike is detected (e.g., patient sneezes), the stimulation can be increased for a short time period to help the patient (for example at 580). Under circumstances where pressure has been gradually increasing over time, and it is identified that the patient's activity level has substantively decreased and the patient's posture has changed from standing to sitting (collectively referenced at 582), it can be automatically determined or estimated that the patient intends to void; in response to this determination, stimulation is stopped or decreased so as to not impede the patient from voiding. An alternative stimulation generated by some algorithms of the present disclosure responsive to the monitored parameters of FIGS. 29A-29C is provided in FIG. 29E. Stimulation can be dynamically provided, for example at 584 (responsive to or triggered by pressure change) and 586 (responsive to or triggered by activity).

Some algorithms of the present disclosure can operate on a closed loop basis. For example, some algorithms can be programmed to detect or identify a positive dp/dt of a predetermined or specific magnitude or threshold (e.g., time/duration) and trigger delivery of stimulation for a fixed duration or alternatively continuously until the detected or identified dp/dt is negative or non-positive for a predetermined time period. Alternatively or in addition, some algorithms can be programmed to detect or identify motion or activity of a predetermined or specific magnitude or threshold (e.g., cough, jump, etc.) and trigger delivery of stimulation for a fixed duration. Alternatively or in addition, some algorithms can be programmed to automatically increase sensitivity (e.g., lowering dp/dt threshold and/or duration requirements) to in turn trigger delivery of stimulation earlier as background pressure increases. Alternatively or in addition, some algorithms can be programmed to increase duration and/or strength of delivered stimulation based on magnitude of detected pressure change and/or background pressure level.

Some algorithms of the present disclosure can operate on an open loop base alone, or in combination with one or more closed loop algorithms, such as the closed loop algorithms described elsewhere in the present disclosure, for example when applied to a single nerve target or electrode set. For example, some algorithms of the present disclosure can be programmed to effect stimulation at a sub-muscular recruitment level continuously, alternatively with an on/off duty cycle (e.g., 30%-70%), to treat OAB and/or fecal incontinence symptoms.

Some algorithms of the present disclosure can operate based on void detection. For example, some algorithms can be programmed to determine or identify an intent to void (e.g., review information implicating an intent to void such as void detection, patient feedback, etc.) and operate to disable delivery of stimulation or other therapy intended to treat stress incontinence. Some algorithms of the present disclosure can be programmed to determine voiding or an intent to void based upon a sustained increase in pressure. While the dp/dt review may trigger stimulation initially, this approach can allow the sustained pressure of voiding (minimal dp/dt) to occur without stimulation. In related embodiments, the systems of the present disclosure can be programmed or formatted to allow a patient to operate a patient device (e.g., press a button) to suppress stress therapy stimulation for a set period of time, for example 5 minutes or 15 minutes.

Returning to FIGS. 1-3, with some embodiments of the present disclosure, the control portion 70 is programmed to reduce or end the delivery of stimulation energy otherwise being applied to activate the external urethral sphincter 34 and/or the external anal sphincter or other muscle for treating stress incontinence (e.g., in one or both of the dynamic and basal modes described above) when the patient desires to void (e.g., where the system is operating to activate the external urethral sphincter 34 to prevent urinary leakage, the delivered stimulation energy can be reduced or ended to permit the external urethral sphincter 34 to relax allowing the patient to more easily void the bladder when desired). With this in mind, in some example systems and methods of the present disclosure, a remote control (e.g., the external device 68) is provided to the patient, allowing the patient to indicate that voiding is desired as described below, with the control portion 70, in turn, being programmed to reduce or end the delivery of stimulation energy. In other embodiments, the systems and methods of the present disclosure can provide at least partial control over therapy such as when a remote control is not available. Such partial control can include at least pausing therapy, starting therapy, stopping therapy and the like. With this in mind, in some examples, the control portion 70 can include or is programmed to include an activation engine that operates in response to information sensed by the sensor 62. For example, with some non-limiting embodiments in which the sensor 62 is or includes an accelerometer, the activation engine can be programmed to operate in response to the patient (or a caregiver) tapping on the patient's body in a region of the implanted sensor 62 a certain number of times within a configurable time period (e.g., three strong taps within two seconds). In some examples, this physical control may act as an alternative therapy deactivation mechanism, such as when the IPG 64 is accidentally activated and/or when the patient desires to void.

In related embodiments, the control portion 70 is programmed to provide (or not provide) stimulation energy at selected levels in response to other volition control scenarios. For example, the control portion 70 can be programmed to operate in an exercise mode upon prompting by the patient or a caregiver (e.g., via the external device 68). The exercise mode can include the provision of stimulation energy to targeted nerve(s) and/or muscle(s) while the patient engages in certain exercises intended improve incontinence (e.g., Kegel exercises).

In yet other embodiments, the control portion 70 is programmed to reduce or end the delivery of stimulation energy otherwise being applied to activate the external urethral sphincter 34 and/or the external anal sphincter or other muscle for treating stress incontinence (e.g., in one or both of the dynamic and basal modes described above) upon self-determining that the patient desires to void. The determination of desired voiding can be based upon information from the sensor 62 and/or other sensors providing information that indicates a desire to void (e.g., a voluntary or involuntary attempt to relax one or more of the sphincters 32, 34, contract the detrusor muscle 30, etc.). With these and related embodiments, the control portion 70 can be programmed to distinguish between a desired void event and a stress event (e.g., the delivery of stimulation energy to activate the external urethral sphincter is not decreased or stopped when the patient sneezes). In yet other optional embodiments, the control portion 70 can be programmed to provide an alert (e.g., via the external device 68) upon determining or sensing a situation where voiding should happen (e.g., upon sensing that the bladder 10 is full). With these and related embodiments, a patient who might otherwise be un-aware of a need to void (for example due to poor health) is alerted of the circumstances (as is his or her caregiver in some embodiments). In yet other optional embodiments where the system includes or incorporates a motion-based transducer sensor (e.g., accelerometer sensor such as a three axes accelerometer as described above), the systems and methods of the present disclosure can include the control portion 70 being programmed to identify or recognize circumstances indicative of the patient desiring to void based upon posture and/or movement. For example, the control portion 70 can be programmed to identify certain movements by the patient in the middle of the night (e.g., the patient is determined to have arisen from a reclined position and/or is moving) as indicating the patient moving toward the bathroom and thus desires to void. Under these and similar circumstances, the control portion 70 can further be programmed to automatically reduce or suppress the delivery of stimulation energy otherwise being applied to activate the external urethral sphincter 34 and/or the external anal sphincter or other muscle for treating stress incontinence.

In other embodiments, the control portion 70 can be programmed to automatically suppress incontinence treatment stimulation energy upon determining that the patient's bladder 10 is full or nearly full (e.g., via any of the techniques described in the present disclosure) and determining a desire or need to void (e.g., based upon reference to one or more of EMG activity, relevant parasympathetic nerve activity, bioimpedance information, etc.). In related embodiments, the control portion 70 can be programmed to identify or recognize circumstances under which the patient is likely to be attempting to void and is experiencing difficulties in achieving a complete or nearly complete voiding of the bladder 10. For example, based upon one or more of the techniques described above, the control portion 70 can be programmed to recognize or identify an attempt to void or desired voiding event (e.g., that the patient's bladder 10 is full or nearly full, followed by a decrease in volume over a relatively short period of time); under these circumstances, the control portion 70 can be further programmed to monitor the desired voiding event, identifying a possible incomplete void. For example, upon identifying the onset of a desired voiding event, the control portion 70 can monitor or estimate a volume of the bladder 10 as well as the change in volume over time; where the change in volume over time decreases below a predetermined threshold or absolute value (or some other comparison indicating that the patient's attempt at voiding is slowing down or nearing completion), the control portion 70 can be programmed to compare the current volume of the bladder 10 with a baseline or “empty” volume value. Where the determined current bladder volume exceeds the baseline or empty volume value by a predetermined amount or percentage, the control portion 70 can be programmed to recognize or designate that the patient is experiencing difficulties in achieving a complete or nearly complete void. Under these circumstances, the control portion 70 can be further programmed to automatically assist the patient in achieving a complete or nearly complete void. For example, the control portion 70 can be programmed to prompt the delivery of stimulation energy formatted to contract the detrusor muscle 30, prompt the delivery of stimulation energy formatted to increase parasympathetic drive, prompt the delivery of stimulation energy formatted to suppress relevant sympathetic nerve activity, deliver high frequency stimulation to relax the pelvic floor muscles, etc.

In yet other embodiments, the systems and methods of the present disclosure optionally provide for stimulation therapy intended to facilitate desired voiding by the patient (e.g., in response to a patient prompt, based in whole or in part upon sensed information, etc.), for example by the control portion 70 being programmed to suppress or reduce delivery of stimulation energy intended to activate or contract the external urethral sphincter 34 as described above. Additionally or alternatively, the systems and methods of the present disclosure can include stimulating one or more of the hypogastric nerves at the T or L level or other nerve of the sympathetic nervous system relevant to bladder control and/or anal control (e.g., sympathetic nerves from T11, T12-L1, L2) in a manner that suppresses the relevant sympathetic nerve drive to thus encourage the natural micturition reflex (e.g., the body's natural, unconscious or reflexive control over voiding is suppressed). In optional related embodiments, the systems and methods of the present disclosure can include the control portion 70 being programmed, under circumstances where voiding is desired, to prompt the delivery of stimulation to target nerve(s) responsible for driving voiding such as the detrusor muscle, directly activing those muscle(s) while relaxing those intended to prevent accidental leakage. This optional approach may be beneficial for patients with incomplete control over the pelvic floor, such as patients who are convalescent, have spinal cord injury, are unconscious, etc.

From the discussions above, some of the treatment systems and methods of the present disclosure can be formatted or implemented to treat patients suffering from urinary retention (the inability to completely empty the bladder when urinating). For example, in some embodiments, the control portion 70 can operate an algorithm programmed to predict or recognize an intent or desire by the patient to void. This recognition can be based upon sensor information and/or patient input, for example by determining that the patient is currently sedentary/not moving, and/or that the patient has assumed a position or posture indicative of an intent to void (e.g., a female patient is determined to likely be in a sitting position), and/or that the patient's bladder or abdominal region has experienced a slow/steady pressure increase over a designated length of time. Once it has been determined or detected that the patient desires to void, the control portion 70 can be further programmed to deliver high frequency stimulation, for example stimulation at levels on the order of 35-50 Hz, to the pudendal nerve or other targeted nerve that in turn allows muscle(s) otherwise associated with prevention of voiding to relax.

In yet other embodiments, the control portion 70 operates one or more algorithms programmed to adjust or modulate or reduce the delivery of stimulation energy otherwise being applied to activate the external urethral sphincter 34 and/or the external anal sphincter or other muscle for treating stress incontinence (e.g., in one or both of the dynamic and basal modes described above) when the patient desires to sleep (thus providing stimulation at a more comfortable level conducive to sleeping). In these and related embodiments, the control portion 70 can be further programmed to detect or determine that the patient intends to sleep or is currently sleeping. For example, sensor information indicating that the patient is sedentary (or has been sedentary for a predetermined length of time) and/or that the patient is prone (or has been prone for a predetermined length of time) can be interpreted as an indication that the patient intends to sleep or is currently sleeping. Additional information, such as time of day, day of week, or past history can further inform the control portion 70 in determining or designating that the patient intends to sleep or is currently sleeping. Alternatively or in addition, the control portion 70 can be programmed to receive sleep-related input information from the patient. Once a determination has been made that the patient intends to sleep or is currently sleeping, the control portion 70 can be programmed to automatically operate a sleep regimen in which delivered stimulation is adjusted to a more comfortable level in some embodiments.

Any of the algorithms described herein can be useful with systems and methods of the present disclosure for treatment of a bowel-related malady, such as fecal incontinence (or bowel incontinence). In some embodiments, for example, systems and methods of the present disclosure for the treatment of fecal incontinence can include electrically stimulating the pudendal nerve (and/or other nerves affecting a bowel-related muscle) to, for example, activate the external anal sphincter. With these and related embodiments, the control portion 70 can be programmed to operate an algorithm that considers sensor information relating to or implicating bowel motility activity (e.g., bioimpedance sensor, pressure sensor located along the small intestine, the large intestine, the rectum, etc.), patient input, etc. The so-provided bowel motility sensor information can dictate adjustments to current stimulation settings (e.g., if it is determined that sensed bowel motility is below a threshold value (thus indicating a lower likelihood of required external anal sphincter activation), a level of basal stimulation can be decreased), an increase in delivered stimulation (e.g., sensed bowel motility over a threshold value and/or over a predetermined time period can be interpreted as requiring increased external anal sphincter activation), etc.

Some aspects of the present disclosure provide systems and methods for titration of a stimulation therapy plan. For example, an overall titration or adjustment plan for mixed incontinence can include increasing stimulation until patient can feel it. This stimulation level (or a level just below the patient's sensation threshold) can be used for open loop therapy. Alternatively or in addition, stimulation can be increased until contraction of the pelvic floor, urinary sphincter, or anal sphincter is observed or notice by the patient. The so-identified stimulation level can then be used as the starting stimulation strength for the functional/phasic closed loop stimulation. In other embodiments, the patient can adjust stimulation based on comfort or outcomes over time. In yet other embodiments, the control portion 70 can be programmed to auto-titrate based on OAB and stress symptoms that can be detected and/or reported depending upon external sensor(s) utilized by the patient. Some titration methods of the present disclosure can include remote monitoring. For example, a voiding diary feedback can be integrated with therapy use information and reported patient comfort data and sensor information (if available). In other embodiments, remote monitoring can be utilized to facilitate automatic ordering/delivery of supplies such as pads and track orders over time to assess therapy efficacy.

### External Device

As mentioned above, the systems of the present disclosure can optionally include one or more external devices 68. As a point of reference, the IMD 60 can be configured to interface (e.g., via telemetry) with a variety of external devices. For example, the external device 68 can include, but is not limited to, a patient remote, a physician remote, a clinician portal, a handheld device, a mobile phone, a smart phone, a desktop computer, a laptop computer, a tablet personal computer, etc. The external device 68 can include a smartphone or other type of handheld (or wearable) device that is retained and operated by the patient to whom the IMD 60 is implanted. In another example, the external device 68 can include a personal computer or the like that is operated by a medical caregiver for the patient. The external device 68 can include a computing device designed to remain at the home of the patient or at the office of the caregiver. Telemetry communication protocols are implemented in hardware and software, carried for example, by the IMD 60 and the external device 68. Standardized telemetry communication technology or protocol that can be used by one or more entities, in an open source or licensed arrangement. For example, Bluetooth®, Bluetooth® low energy (BLE), near-field magnetic induction (NFMI) communication, Wi-Fi, Zigbee®, etc.

In some embodiments, the external device 68 can be programmed (e.g., operate an installed software application) to provide a clinician with the ability to program various operational modes of the IMD 60. For example, where the external device 68 serves as a clinician programmer, the clinician can enter various performance attributes or protocols (e.g., stimulation levels, frequency, timing, etc.) that are operated upon by the IMD 60 (e.g., via the control portion 70).

In some embodiments, the external device 68 can be programmed (e.g., operate an installed software application) to provide a patient with the ability to control or adjust operation of the IMD 60. For example, where the external device 68 serves as a patient remote, the patient can be afforded the ability to adjust stimulation parameters in an effort to balance patient comfort with treatment efficacy. In addition or alternatively, the external device 68 can provide the patient with the ability to switch between pre-set groups of therapy parameters (e.g., the control portion 70 can be programmed to deliver stimulation energy at a first level in a first mode of operation and at a second level in a second mode of operation, the external device 68 can afford the patient the ability to select between the first and second modes of operation). With these and related embodiments, the external device 68 can provide the patient with the ability to stop the delivery of stimulation energy (or switch between modes of operation) to better ensure that the patient can void when desired and that the dynamic leak suppression therapy (for example) does not interfere with desired, normal voiding.

In one non-limiting example where the external device 68 is configured to serve as a patient remote, the patient can enter information at the external device indicating that an incontinence-related event, for example leakage, has just or very recently occurred. This event information is signaled to the control portion 70 that in turn is programmed to act upon the event information. For example, the control portion 70 can be programmed such that when otherwise operating to prompt delivery of stimulation energy intended to treat incontinence and a signal is received from the patient that a leakage event (or other incontinence event) has just or very recently occurred, the control portion 70 alters or increases the stimulation energy being delivered (e.g., increased amplitude, increased speed of response, increased duration of stimulation, etc.) in an effort to better address the patient's current needs. With these and related embodiments, the control portion 70 can effectively be programmed to prompt the delivery of less than “full strength” stimulation for treating incontinence in a normal mode of operation, and then increase or deliver “full strength” stimulation only when being informed by the patient of a need for such stimulation.

In yet other non-limiting embodiments, the systems and methods of the present disclosure can provide cloud based patient management (e.g., the external device 68 interfaces with the IMD 60 via the internet, Wi-Fi, etc.). With these and related embodiments, the systems and methods of the present disclosure can facilitate remote review or monitoring by a clinician of the therapy behavior being provided to the patient (e.g., dynamic event trigger) and/or the outcome of delivery therapy. Additionally or alternatively, the systems and methods of the present disclosure can facilitate a clinician remotely providing additional therapy instructions/protocols/modes of operation to the IMD 60, delete an existing programmed therapy delivery mode of operation, and/or modify parameters of an existing programmed therapy delivery mode of operation.

In yet other non-limiting embodiments of the present disclosure, the external device 68 can be programmed (e.g., operate an installed software application) that provides the patient with the ability to document information of interest, such as voiding and/or leaking events. The so-recorded information can be utilized by a clinician to assess therapy outcomes. In this regard, the clinician can review recorded information directly on the external device 68, or the recorded information can be delivered to a separate device (e.g., via the cloud) for clinician review. Where, for example, leakage events are reported by the patient on a patient remote, the so-reported information can be captured in a log such that an estimate of stimulation therapy effectiveness can be made.

In some embodiments, the control portion 70 is configured to provide the patient with the ability to temporarily de-activate the IMD 60 from delivering stimulation signals, for example via the external device 68.

### UUI

Some example systems and methods of the present disclosure incorporate features (e.g., system components, programming, etc.) for treating UUI, for example by providing electrical stimulation to a targeted nerve, muscle, or other tissue at levels appropriate for reducing the sensation of a need or urge to empty the bladder 10 at times when the bladder 10 is not actually full or nearly full. With these and related embodiments, the systems and methods can include estimating or measuring or determining a current volume of the bladder 10 (e.g., information indicative of a current volume of the bladder 10 can be obtained by the sensor 62, and compared against predetermined bladder volume levels obtained, for example, by urodynamic assessment), along with patient-provided feedback of a perceived bladder volume or “urge” to void. Under circumstances where the estimated current volume of the bladder 10 is less than full yet the patient perceives a strong urge to void, the control portion 70 can be programmed to prompt the provision of appropriate stimulation energy to the targeted nerve and/or muscle sufficient to reduce the sensation of a need or urge to void. The control portion 70 can be further programmed to modulate delivered stimulation (e.g., increase intensity, delivery time, etc.) over time as the current volume of the bladder 10 increases (e.g., as determined, for example, from information provided by the sensor 62).

In related embodiments, the UUI treatment systems and methods of the present disclosure can include obtaining EMG information relative to the detrusor muscle 30 as described above. Current detrusor EMG information can, in turn, be correlated or useful to estimate current bladder volume, for example with reference to urodynamic assessment information of the patient. Under circumstances where the current detrusor EMG information indicates that the bladder 10 is less than full yet the patient is perceiving an urge to void (e.g., patient-provided feedback), the control portion 70 can be programmed to prompt the provision of appropriate stimulation energy to the targeted nerve and/or muscle sufficient to reduce the sensation of a need or urge to void. The control portion 70 can be further programmed to modulate delivered stimulation (e.g., increase intensity, delivery time, etc.) over time as the detrusor EMG information (and thus estimated bladder volume) increases. Alternatively or in addition, EMG information relative to the pelvic floor muscles 18 can be obtained and used as the basis for UUI stimulation treatment. For example, pelvic floor EMG information can be correlated with bladder volume. Under circumstances where the current pelvic floor EMG information indicates that the bladder 10 is less than full, yet the patient is perceiving an urge to void (e.g., patient-provided feedback), the control portion 70 can be programmed to prompt the provision of appropriate stimulation energy to the targeted nerve and/or muscle sufficient to reduce the sensation of a need or urge to void. The control portion 70 can be further programmed to modulate delivered stimulation (e.g., increase intensity, delivery time, etc.) over time as the pelvic floor EMG information (and thus estimated bladder volume) increases.

In related embodiments, the UUI treatment systems and methods of the present disclosure can include obtaining bioimpedance information indicative of a position of the pelvic floor muscles 18. The pelvic floor bioimpedance information can be correlated with bladder volume (e.g., via urodynamic assessment), and used as the basis for UUI stimulation therapy as described above.

The UUI treatment systems and methods of the present disclosure can optionally include sensing or detecting parasympathetic nerve activity otherwise indicative of an urge or desire to void the bladder 10 (e.g., via eletroneurography (ENG) techniques), for example by an electrode or other sensor component along one or more of the hypogastric nerves or the pelvic splanchnic nerves at the T or L level. With these and related embodiments, the control portion 70 can be programmed to detect or recognize parasympathetic nerve activity relevant to the bladder 10 and, under circumstances where relevant parasympathetic nerve activity is detected yet the bladder 10 is determined or estimated to be less than full, prompt the delivery of stimulation energy appropriate to suppress the parasympathetic nerve activity.

In some examples, the UUI treatment systems and methods of the present disclosure can include providing the patient with the external device 68 as described above, configured to provide the patient with the ability to select from two (or more) modes of operation. For example, the external device 68 can be configured to allow the patient to select or switch between an auto control mode and a continuous/open loop mode, with the selected mode of operation being signaled to the control portion 70 that in turn is programmed to prompt a provision of a corresponding UUI stimulation therapy or protocol. In the auto control mode, sensor feedback (e.g., as described above) is employed to determine whether or not stimulation energy is delivered and, where UUI stimulation energy is to be delivered, the format (e.g., level, timing, etc.) of such stimulation. In the continuous/open loop mode, UUI stimulation energy is delivered without reference to feedback information.

The UUI treatment systems and methods of the present disclosure can optionally include providing additional feedback to the patient via the external device 68, for example feedback information indicating a determination that the patient bladder 10 is full or nearly full (e.g., the control portion 70 is programmed to estimate bladder volume via one or more of the sensor-based parameters as described above such as detrusor EMG, pelvic floor EMG, bioimpedance, etc.). The patient, in turn, can understand from this feedback information a need to void his/her bladder and take appropriate action. In related embodiments, the external device 68 can be configured to provide the patient with an ability to better or more easily void his/her bladder when desired (e.g., after reviewing feedback information indicating that his/her bladder is full or nearly full, the patient can locate a bathroom or other locale where voiding is appropriate, and then operate the external device as described below). For example, the external device 68 can provide a button, switch, or the like, the actuation of which causes the control portion 70 to prompt the delivery of stimulation energy appropriate for activating or increasing a parasympathetic drive to void the bladder 10. Alternatively or in addition, the external device 68 can be configured such that the patient can cause the control portion 70 to prompt the reduction or suppression of incontinence stimulation energy (e.g., with optional embodiments in which the system is configured to provide incontinence stimulation therapy, for example delivering stimulation energy formatted to cause the external urethral sphincter 34 to activate or contract (for example by delivering stimulation energy to the pudendal nerve 44), the delivery of this incontinence stimulation energy can be reduced or suppressed when the patient is ready to void). In other, related embodiments, the systems and methods of the present disclosure can include informing the patient at the external device 68 of a need to void his/her bladder. The external device 68 can further request or otherwise facilitate the patient entering information indicating that he/she is currently ready to void; the control portion 70 can be programmed to automatically take action(s) that promote voiding (e.g., delivering stimulation energy formatted to contract the detrusor muscle 30, delivering stimulation energy formatted to increase parasympathetic drive, suppressing stimulation energy formatted to treat incontinence (e.g., suppressing stimulation energy otherwise formatted to cause the external urethral sphincter 34 to contract, etc.)).

In some embodiments, the UUI treatment systems and methods of the present disclosure can include an algorithm operated by the control portion 70 that is programmed to detect or predict that the patient is experiencing or suffering from elevated UUI symptoms. For example, the algorithm can review sensed bladder peak volumes and number of voids over time. Where it is determined (e.g., based upon a comparison with predetermined limits) that the patient is frequently voiding and does not normally wait until his/her bladder is near a “full” capacity before voiding, the algorithm can designated that the patient is experiencing UUI (or overactive bladder). In related embodiments, the control portion 70 can further be programmed to operate in a UUI heightened treatment mode upon determining that the patient is suffering from heightened UUI symptoms, for example delivering stimulation at a higher duty cycle or amplitude.

The UUI treatment systems and methods of the present disclosure can further implements artificial intelligence or machine learning features. For example, through artificial intelligence or machine learning techniques, the control portion 70 can develop a customized therapy and/or sensing and diagnostics for the particular patient. With these and related embodiments, therapy titration could be simplified and could minimize or even eliminate the need for training with the help of urodynamic assessment.

### Therapy Screening and Trialing

In some example systems and methods of the present disclosure, stimulation parameters associated with one or more modes of operation provided by the control portion 70 can be generated as part of a trial or trialing protocol. For example, the systems and methods of the present disclosure can optionally include delivering a stimulation element, such as the stimulation element 66, to a target location, and connecting the stimulation element to an external stimulator (e.g., a pulse generator located external the patient). With this approach, the system can be operated to evaluate therapy response prior to full implant of the IPG 64. The stimulation element 66 can optionally be delivered trans-urethrally, or can be provided as part of a chronic lead with a percutaneous adapter. In related embodiments, the trialing protocol can further include the sensor(s) 62 being implanted at the target location, or can be located external the patient at a position generating information representative of the future implanted sensor. For example, the sensor 62 utilized for the trialing protocol can be the actual sensor to be implanted in the future that is modified and attached to the patient's body with an adhesive, the trialing protocol can include use of an external-based impedance sensor system, etc. In related embodiments, one or more of the sensors utilized for patient trialing can be carried by a garment worn by the patient, for example akin to the garment 372 (FIG. 17B) described above. With other therapy screening, and chronic and trialing systems of the present disclosure, a patient remote is provided (or an app loaded onto a mobile device of the patient) through which patient input is received.

In some examples, methods of patient trialing can include employing a nerve stimulation delivery device configured to be applied or installed to a patient in a minimally invasive manner. For example, a transvaginal probe or the like, a St. Mark's™ disposable pudendal NCS electrode (available from Natus Medical), a FemPulse™ neuromodulation therapy device (available from FemPulse, LLC), a stimulation device (e.g., pudendal nerve stimulation) intended to be delivered transrectally, etc., can be utilized to deliver electrical stimulation (e.g., transvaginal stimulation, transrectal stimulation, etc.) that simulates possible location or placement of an implantable lead assembly intended to stimulate the pudendal nerve. The delivered stimulation as part of the trialing (or for actual treatment) can include using the dynamic modes of the present disclosure in response to elevated abdominal pressure (e.g., sensed or estimated abdominal pressure), constant electrical stimulation delivery, a combination of dynamic and constant stimulation, etc. With these and related embodiments, abdominal pressure can be measured or sensed using a pressure sensitive membrane or sensor delivered, for example, transvaginally or transrectally. The sensor (e.g., pressure sensor, EMG sensor, bioimpedance sensor, accelerometer, etc.) can optionally be integrated into the transvaginal or transrectal stimulation device, along with a controller and power supply (e.g., battery). Further, the so-configured device could be used for trialing as well as a temporary indwelling therapy delivery device. In other embodiments, the abdominal pressure information can be provided via an external device, such as a pressure sensor located along a belt line of the patient configured to measure changes in abdominal pressure such as an effort belt (or “RIP” belt) and/or abdominal EMG.

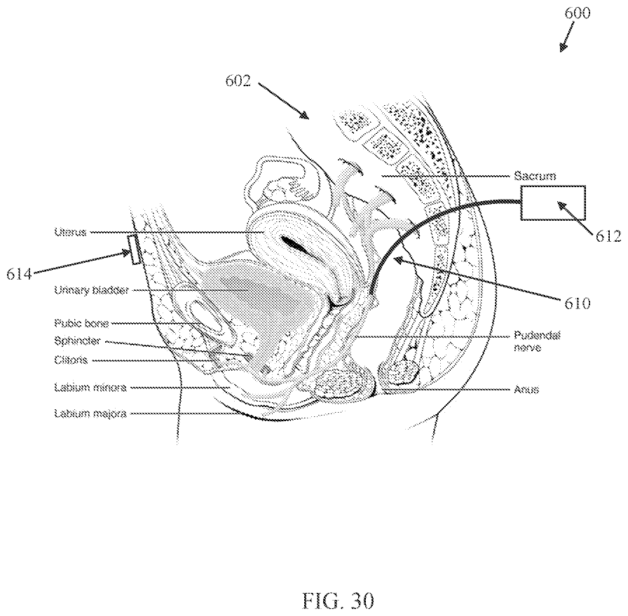

Another non-limiting example of a trialing system 600 in accordance with principles of the present disclosure is shown in FIG. 30 as applied to a patient 602. The system 600 includes a lead assembly 610, a pulse generator 612, and optionally one or more sensors 614. The lead assembly 610 can be, or can be akin to, a percutaneous nerve evaluation (PNE) lead, configured for percutaneous insertion through the patient's skin and carrying one or more stimulation elements. With the example of FIG. 30, the stimulation element of the lead assembly 610 is positioned to apply stimulation energy to the pudendal nerve. In other embodiments, trialing can entail locating the stimulation element at another region of the pudendal nerve differing from that implicated by FIG. 30 and/or at or along a nerve other than the pudendal nerve and/or directly on a muscle or organ. While the lead assembly 610 is illustrated as extending posteriorly through or near the sacrum, other approaches to an intended target site can be employed (e.g., through the buttocks, anteriorly, transvaginal, transrectal, etc.). Also, an anterior approach can be utilized. The lead assembly 610 is electrically connected to the pulse generator 612 that is otherwise located and maintained outside the patient's body. The sensor(s) 614 can take various forms appropriate for sensing information of interest to a particular trialing session. For example, the sensor 614 can be configured for placement outside the patient's body (e.g., on the skin at any location that may or may not be directly implicated by the arrangement of FIG. 30) as in FIG. 30 to measure a parameter indicative of pressure, volume, etc., such as an external adhered sensor or sensor interface to perform therapy during a trialing phase before permanent system implant. In other embodiments, the sensor 614 can be configured to be located inside the patient's body (e.g., placed on or near the bladder to measure a parameter indicative of pressure, volume, etc.) and can be delivered in various manners. Further, while the sensor 614 is depicted as being separate from the lead assembly 610, in other embodiments, the sensor 614 can be carried by the lead assembly 610. Regardless, in some embodiments, test stimulation for a trialing phase (e.g., for closed loop therapy, stress incontinence, mixed closed loop/open loop therapy, etc.) can include placement of a temporary or permanent lead as discussed. If a permanent lead is provided, a percutaneous extension can be utilized, tunneling about 8-12 inches to an alternate percutaneous site to prevent infection of the permanent lead during trialing. An external adhered sensor or sensor interface can be utilized with the trialing phase before permanent system implant.

In some optional, related embodiments, a physician programmer (“system”) that assists the implanting physician with identifying the optimal (trial) lead location is provided, for example leveraging the direct correlation between perineal EMG and EUS contraction to optimize lead placement and the PNS therapy. This is achieved by providing the system with inputs from any combination of: perineal EMG, urethral pressure, and feedback from the patient. Perineal EMG activity and an increase in urethral pressure both help identify external urethral sphincter contraction. To collect the EMG data, the system could connect to existing EMG machines or the system could integrate EMG processing (and filtering) and therefore connect directly to EMG patches. The same could be true for collecting the urethral pressure data (data connection or pressure port). The patient feedback could come in the form of verbal feedback regarding sensations (manually entered in to the system by the implanting team) or connected button(s) that could be depressed by the patient per instructions from the physician. The system could run algorithms to cycle through different stimulation parameters and electrode configurations while monitoring these inputs, and then provide visual (on system screen) or auditory signals to the implanting physician as optimal lead location(s) are identified by the system.

In some optional, related embodiments, the external evaluation/trial neurostimulator includes EMG capabilities to allow for the capture of EMG data during the evaluation period. Perineal patches attached by the physician or patient would be connected to the trial neurostimulator and worn as much as feasible during the trial. The EMG data would inform the system of the effectiveness of the closed-loop PNS system at contracting the EUS during the trial period. This data could be exported at the conclusion of the trial or throughout the trial via different means of internet connectivity. The trial system could also adjust electrode configuration and stimulation parameters throughout the evaluation period via algorithms that consider this EMG input, other planned system inputs (pressure, acceleration, etc.), and feedback from the patient on the occurrence of leaks or sensations/discomfort. The objective being to help identify optimal stimulation parameters during evaluation, improve the effectiveness of the evaluation system, and perhaps help identify patient-specific/variable therapy configurations.

In some examples, methods of therapy screening and/or trialing can include conventional urodynamic testing with Leak Point Pressure (“LPP”) evaluation in conjunction with electrical stimulation therapy delivered via, for example, a transvaginal or transrectal electrical stimulation device (or other minimally invasive configuration) arranged to simulate possible location or placement of an implantable lead assembly intended to stimulate the pudendal nerve (or other target structure), or a temporary indwelling or permanent implanted system. The electrical stimulation delivered to the pudendal nerve (or other target structure) could be assessed with respect to changes in LPP while the patient is coughing or experiencing some other elevated abdominal pressure event that may otherwise cause SUI leakage. For example, an improvement in LPP could be correlated to effective treatment of SUI and be used as an indication of effectiveness of the therapy and a decision regarding acceptability of the therapy for the particular patient.

In some examples, methods of therapy screening and/or trialing can include specialized urodynamic testing with Urethral Pressure Profile (“UPP”) evaluation in conjunction with electrical stimulation therapy delivered via a transvaginal or transrectal electrical stimulation device arranged to simulate possible location or placement of a lead intended to stimulate the pudendal nerve (or other target structure), or a temporary indwelling or permanent implanted system. As a point of reference, UPP is a manipulation test of the bladder neck, urethra, and its sphincters. UPP measures the balance of pressure at each point along the urethra while a small amount of distending fluid is instilled continuously as an indicator of possible voiding dysfunction. The UPP evaluation could assess changes in urethral pressure exerted on a urethral catheter at all specific locations along the urethra. The change in UPP while stimulation is being delivered versus when stimulation is off could be correlated to effective treatment of SUI and be used as an indication of effectiveness of the therapy and a decision regarding acceptability of the therapy for the particular patient. For example, the transvaginal or transrectal electrical stimulation device can deliver pudendal nerve stimulation while a pressure sensing catheter is slowly traversed through the urethra while stimulation is cycled on/off. The increase in urethra pressure (“stimulation on” response) is almost instantaneous and can be compared to the “stimulation off” pressures. Furthermore, the actual locations along the urethra that experience improvements in pressure may be an indicator of potential effectiveness as a chronic treatment. For instance, an improvement in the area of the external urethral sphincter may be found to be more effective if the improvements in urethral pressure only exist in the area of the proximal urethra.

In some embodiments, the urodynamic testing or evaluations described above could be performed with the patient's bladder in an empty state and in a stressed condition where the stressed condition would be represented by degrees of fullness or filling. In some embodiments, saline could be incrementally filled or infused into the patient's bladder, for example in increments of 50 ml or 100 ml, from empty to maximum capacity. In some embodiments, a determination of a maximum natural filling level via these and other techniques as part of a patient training session(s) (that may or may not include stimulation being applied during the fill test) can be used to improve algorithm responsiveness upon final implant. In some embodiments, a determination of a minimum natural bladder filling level can be determined via these and other techniques as part of a patient training session(s) (that may or may not include stimulation being applied) can be used to improve algorithm responsiveness upon final implant (e.g., algorithms employed to address retention issues can benefit from a minimum fill level of the patient).

In some examples, methods of patient trialing can include assessing a best sensor type and/or location for obtaining useful abdominal pressure (or other parameter) information with a permanently implanted system by collecting a series of sensing signals representing intraabdominal pressure during the trialing period (e.g., in an acute in-lab procedure, a temporary indwelling trial, etc.). With these and related embodiments, a reference pressure sensor can be employed such that other signals are compared to the reference pressure sensor data in order to determined which permanently implanted signals will be most effective or beneficial. The reference sensor can be, for example, one or more of anal rectal manometry, transvaginal manometry, surface EMG, RIP effort belt, accelerometer, etc. With these and related embodiments, the various surrogate signals can be assessed or compared to a reference signal while the patient is directed to a variety of bodily positions and/or is prompted to experience or replicate one or more events that affect intraabdominal pressure such as coughing, Valsalva maneuver, jumping, lifting, etc. In yet other related embodiments, the therapy device could be programmed to use different surrogate signals for different body positions (e.g., standing, laying, sitting, etc.), bladder volumes and/or different intraabdominal pressure events such that the highest probability of utilizing the most effective pressure signal can be achieved.

In some examples a method comprises delivering, via the stimulation element 66 and during a treatment period, stimulation to a targeted nerve and/or muscle to treat incontinence and/or UUI. In some examples, the treatment period may comprise a period of time beginning with the patient turning on the therapy device and ending with the patient turning off the device. In some examples, the treatment period may comprise a selectable, predetermined start time (e.g. 6 a.m.) and selectable, predetermined stop time (e.g. 10 p.m.).

In some examples, the stimulation is applied during some of the treatment period without being delivered throughout the treatment period. Stated differently, in some examples stimulation may be performed during some portions of the treatment period but not during other portions of the same treatment period. In some examples, stimulation applied during the treatment period may comprise stimulation delivered throughout the treatment period. In some such examples, stimulation delivered throughout the treatment period may comprise stimulation being delivered throughout the entire treatment period. In some such examples, the term “throughout the entire” may comprise stimulation being performed in 100 percent of the treatment period. However, it will be understood that in some examples some startup routines, shutdown routines are not considered part of the 100 percent.

In some examples, stimulation being delivered throughout the treatment period comprises stimulation delivered throughout substantially the entire treatment period. In some such examples, in this context, the term “substantially the entire” comprises at least 70 percent, at least 80 percent, at least 90 percent, or at least 95 percent of the entire treatment period.

In some such examples, stimulation of the targeted nerve and/or muscle which is maintained during the treatment period may be referred to as being “on-going” in the treatment period but not continuous. For instance, the on-going stimulation may be implemented via a duty cycle, train of pulses, etc. such that the stimulation need not be one hundred percent continuous. Rather, in some such examples, the term “on-going” stimulation may refer to stimulation which does not start and/or stop based on occurrence of some event such as a controller signal to start stimulation or a controller signal to stop stimulation.

In some examples, stimulation of the targeted nerve and/or muscle may be performed via open loop stimulation. In some examples, the open loop stimulation may refer to performing stimulation without use of any sensory feedback of any kind relative to the stimulation.

Conversely, in some examples, stimulation of the targeted nerve and/or muscle may be performed via closed loop stimulation. In some examples, the closed loop stimulation may refer to performing stimulation at least partially based on sensory feedback regarding parameters of the stimulation and/or effects of the stimulation as described above.

Some of the systems and methods of the present disclosure have been described primarily as treating urinary incontinence in that they mitigate a stress urinary incontinence leak through functional stimulation in response to a pressure increase event. In other embodiments, the systems and methods of the present disclosure can optionally provide for or additionally include providing a therapeutic benefit through training of relevant muscles. For example, a training protocol or therapeutic mode can be programmed/implemented whereby stimulation is delivered on a schedule for creating muscle contraction of the sphincter and/or other continence muscles as a form of training, thereby providing a therapeutic effect of increasing continence muscle effectiveness. In some embodiments, the system is configured and programmed to operate in a treatment mode, a therapeutic mode, or both.

### Other Sensing Modalities

In some embodiments, the systems and methods of the present disclosure can include or incorporate one or more sensing modalities in place of, or in addition to, the sensing modalities described above (e.g., the sensing modalities implicated by FIG. 18). As a point of reference, and as described elsewhere, various stimulation therapy algorithms or protocols of the present disclosure can act upon or be responsive to events or scenarios experienced by the patient that are otherwise likely to benefit from the delivery of stimulation and/or change in stimulation currently being delivered (e.g., cough, sneeze, rapid movement, Valsalva, etc.). Predicted onset or occurrence of an event or scenario implicating delivery or (or change in) stimulation can be automatically determined by monitoring one (or more) sensed parameters of the patient. The patient's intra-abdominal pressure (IAP) (e.g., absolute IAP, change in IAP over time, etc.) can oftentimes be a highly viable indicator of an event or scenario of interest. However, the direct sensing of IAP can present certain obstacles and/or complexities. With this in mind, some embodiments described below present sensor formats and locations appropriate for sensing a parameter having a correlation with IAP, and thus are useful as a surrogate for IAP with various ones of the stimulation therapy algorithms and protocols of the present disclosure. Alternatively or in addition, one or more of the sensing modalities below are useful for detecting parameters indicative of a possible SUI inducing event that may be different from change in IAP. For example, local tissue pressure from externally pushing on the bladder may cause a patient to experience a urinary leak but without a distinct increase in IAP.

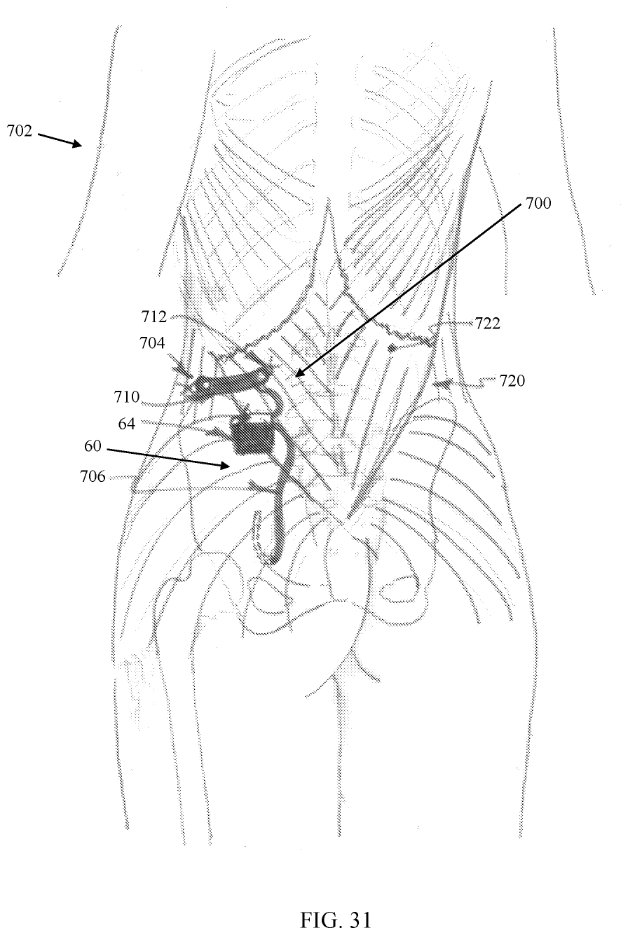

Another sensing modality of the present disclosure, and useful, for example, as a surrogate for direct measurement of IAP (or other SUI causing event), is shown in FIG. 31 as part of a treatment system 700 (referenced generally) implanted within a patient 702. The treatment system 700 can, in many respects, be similar to other treatment systems of the present disclosure, for example the treatment system 50 of FIG. 3. In general terms, the treatment system 700 includes the IMD 60 as described above and a sensor 704. The IMD 60 includes the IPG 64 implanted within the patient 702, and a stimulation lead 706 routed from the IPG 64 to position one or more stimulation elements (e.g., electrode(s)) at a desired stimulation target site. The IPG 64 is programmed (or is prompted) to deliver stimulation signals to the stimulation element via the lead 706 that in turn are applied to the target site. It will be understood that a location of the IPG 64 and of the stimulation lead 706 reflected in FIG. 31 are examples only, and are in no way limiting.

The sensor 704 is configured to be implanted within the patient 702, and can assume various forms. In general terms, the sensor 704 is generally formatted and located to sense or capture changes in a shape or other parameter of the patient's abdomen. A wired connection can be established between the sensor 704 and the IPG 64 and through which information collected by the sensor 704 is delivered to the IPG 64; in other embodiments, the sensor 704 can communicate with the IPG 64 (or other device) via wireless connection. The optional wired connection can be employed to deliver power (if necessary) from the IPG 64 to the sensor 704. In other embodiments, the sensor 704 may be self-powered or may not require power.

With the above in mind, in some examples the sensor 704 can be or can include a strain-type sensor, or strain measuring sensor, placed in a subcutaneous space/intra-abdominally at an anatomical area correlating with IAP. The strain sensor 704 can be sized and positioned to sense or detect a local elongation, change in angle, or other deformation, for example by opposing first and second ends 710, 712 of the strain sensor 704 being anchored at anatomical locations likely to move relative to one another when the abdomen changes shape, for example extending or spanning across muscles groups or structures. In the non-limiting example of FIG. 31, the first end 710 is anchored (e.g., secured by suture or other fixation technique) to tissue of an external oblique muscle 720 and the second end 712 is anchored to lumbodorsal fascia 722. With this arrangement, the strain sensor 704 will readily detect or sense abdominal movements or changes of interest (e.g., an SUI causing event such as cough, sneeze, etc.). In other words, with the arrangement of FIG. 31 (and similar arrangements), the sensing signal generated by the strain sensor 704 will substantively vary with certain abdominal movements or changes, and from which a determination or prediction of the occurrence of an event of interest and/or onset or change in a scenario of interest can be automatically made and acted upon. The strain sensor 704 can be anchored to other locations along the external oblique muscle 720 differing from that of FIG. 31. Further, other musculature apart from or in addition to the external oblique muscle 720 can serve as a viable location for the strain sensor 704. For example, internal oblique, pyramidalis, rectus abdominis, transverse abdominis, intercostals, latissimus dorsi, iliocostalis lumborum serratus posterior inferior, longissimus thoracis, erector spinae, quadratus lumborum, psoas major, psoas minor, etc. In yet other embodiments, muscles, ligaments, tendons and other structures of the pelvic flor and/or perineal location can be a useful location for the strain sensor 704, for example with optional transvaginally-placed treatment systems.

The implant location and orientation of the strain sensor 704 reflected by FIG. 31 is but one example. The strain sensor 704 can be implanted in various other locations and fixed to tissue in various ways that allow the strain measurement to correlate to events that cause SUI leakage events (for example), such as rapid changes in IAP. The strain sensor 704 can be configured and arranged to measure or sense axial strain or angular strain. The strain sensor 704 can be implanted and arranged to leverage the recognition that the muscles enclosing the abdomen will react and contract to contain IAP in a way that will result in local strain. For example, the strain sensor 704 the opposing ends 710, 712 can be anchored to the lumbodorsal fascia 722. As strain is experienced in the lumbodorsal fascia 722, strain will be imparted onto the sensor 704, serving as an input signal that can be correlated with, or determined to be indicative of, an SUI inducing event. In yet other embodiments, the strain sensor 704 can be arranged to extend in a more superior/inferior direction (as compared to the lateral arrangement of FIG. 31), for example to generate information indicative of bending. With these and related embodiments, information from the so-arranged strain sensor 704 can be useful, for example, to rule out certain events (e.g., where other sensor information implicates an increase in IAP (and thus a potential need for stimulation) and the superior/inferior arranged strain sensor 704 indicates that the patient is bending, a decision can be made that the detected increase in IAP is not a SUI inducing event).

The sensor 704 can assume other forms appropriate for sensing strain or displacement. For example, an implantable, tactile (pressure sensitive) sensor can be utilized as, or as part of, the sensor 704, and can be anchored at any of the subcutaneous spaces/intra-abdominal locations mentioned above that can otherwise be highly correlated with IAP. With these and related embodiments, tissue pressure between structures in the abdomen or within an anatomical structure (e.g., abdominal wall muscle) can be sensed/measured, with the tactile sensor 704 generating a characteristic signal that can be reviewed and applied to assess when the patient is experiencing, or is about to experience, an event of interest, such as an SUI event. Thus, signaled information from the tactile sensor 704 can be used to trigger stimulation or terminate the delivery of stimulation, for example stimulation intended to treat SUI. The tactile sensor 704 can be implanted at or within various muscles such as, but not limited to, external oblique, internal oblique, pyramidalis, rectus abdominis, transverse abdominis, intercostals, latissimus dorsi, iliocostalis lumborum serratus posterior inferior, longissimus thoracis, erector spinae, quadratus lumborum, psoas major, psoas minor, pelvic floor muscles and structures, etc. In other embodiments of the present disclosure, the tactile sensor 704 can be positioned in a virtual space of the patient's anatomy, such as naturally exists between anatomical structures (e.g., between fascial layers).

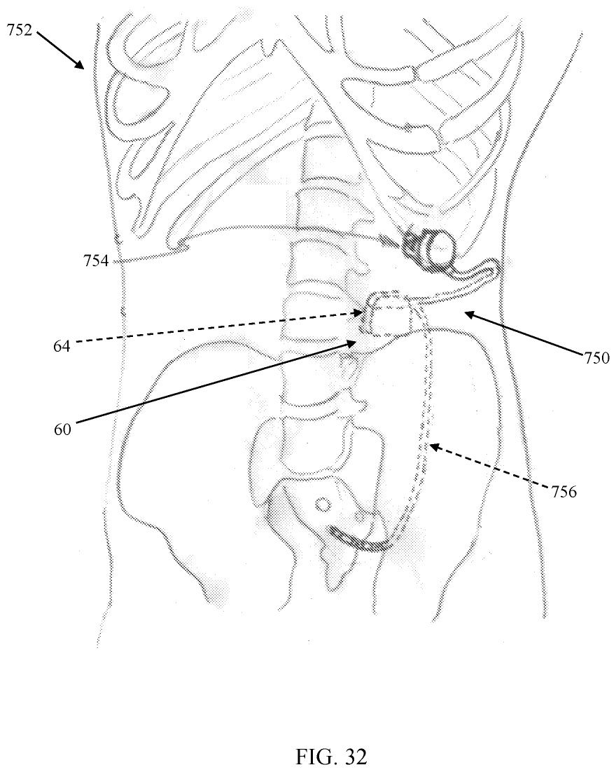

Another sensing modality of the present disclosure, and useful, for example, as a surrogate for direct measurement of IAP, is shown in FIG. 32 as part of a treatment system 750 implanted within a patient 752. The treatment system 750 can, in many respects, be similar to other treatment systems of the present disclosure, for example the treatment system 50 of FIG. 3. In general terms, the treatment system 750 includes the IMD 60 as described above and a sensor 754. The IMD 60 includes the IPG 64 implanted within the patient 752, and a stimulation lead 756 routed from the IPG 64 to position one or more stimulation elements (e.g., electrode(s)) at a desired stimulation target site. The IPG 64 is programmed (or is prompted) to deliver stimulation signals to the stimulation element via the lead 756 that in turn are applied to the target site. It will be understood that a location of the IPG 64 and of the stimulation lead 756 reflected in FIG. 31 are examples only, and are in no way limiting.

With the above in mind, in some examples the sensor 754 can be or can include an implantable proximity-type displacement sensor located in a subcutaneous space/intra-abdominally at an anatomical area correlating with IAP. The proximity-type displacement sensor 754 can incorporate one or more conventional sensing technologies such as inductive, capacitive, optical, magnetic, etc. appropriate for detecting or measuring changes in the distance or position of two anatomical locations in the body. A wired connection can be established between the sensor 754 and the IPG 64 and through which information collected by the sensor 754 is delivered to the IPG 64; in other embodiments, the sensor 754 can communicate with the IPG 64 (or other device) via wireless connection. The optional wired connection can be employed to deliver power (if necessary) from the IPG 64 to the sensor 754. In other embodiments, the sensor 754 be self-powered or may not require power and may communicate wirelessly with other component(s) of the treatment system.

The proximity-type displacement sensor 754 can be implanted so as to detect or measure displacement between muscle groups or anatomical structures or within a muscle. In some non-limiting embodiments, the proximity-type displacement sensor 754 is arranged to detect or measure changes in displacement between lumbodorsal fascia and intercostal muscle fascia. In the example of FIG. 32, one portion of the proximity-type displacement sensor 754 is sutured or otherwise anchored onto intercostal fascia. As a point of reference, in the view of FIG. 32, the proximity-type displacement sensor 754 is anteriorly located (intercostal fascia) whereas the IPG 64 is posteriorly located (dorsal). In some non-limiting examples, then, the IPG 64 can include or carry one or more components that interact with the proximity-type displacement sensor 754 such that the proximity-type displacement sensor 754 measures or senses changes in a distance between the sensor 754 and the IPG 64. Regardless, the proximity-type displacement sensor 754 is sized and positioned to sense or detect a local elongation, change in angle, or other deformation at an anatomical location(s) likely to deform when the abdomen changes shape. With this arrangement, the proximity-type displacement sensor 754 can readily detect or sense abdominal movements or changes of interest (e.g., an SUI causing event such as cough, sneeze, etc.). In other words, with the arrangement of FIG. 32 (and similar arrangements), the sensing signal generated by the proximity-type displacement sensor 754 will substantively vary with certain abdominal movements or changes, and from which a determination or prediction of the occurrence of an event of interest and/or onset or change in a scenario of interest can be automatically made and acted upon. The proximity-type displacement sensor 754 can be anchored to other locations differing from that of FIG. 32.

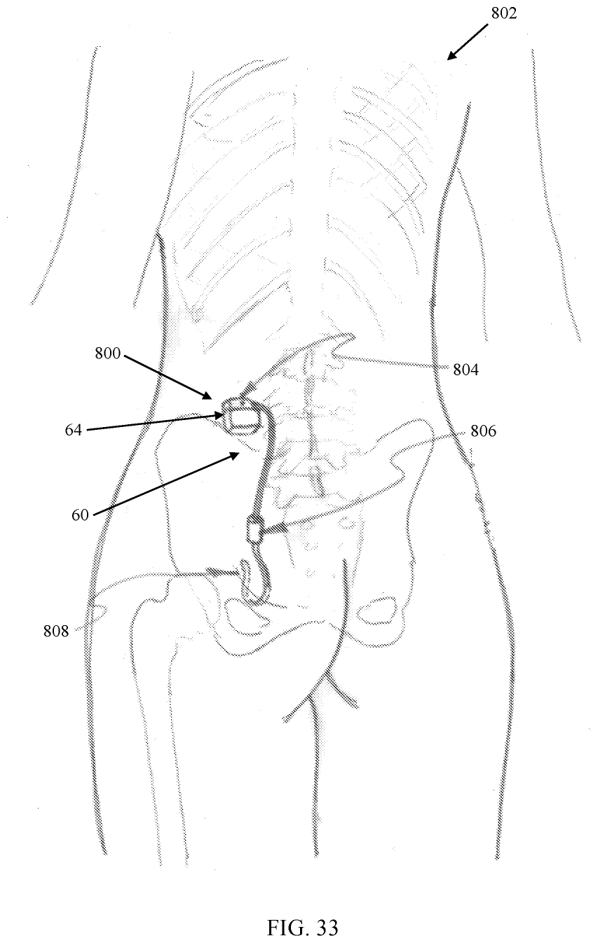

Another sensing modality of the present disclosure, and useful, for example, as a surrogate for direct measurement of IAP, is shown in FIG. 33 as part of a treatment system 800 implanted within a patient 802. The treatment system 800 can, in many respects, be similar to other treatment systems of the present disclosure, for example the treatment system 50 of FIG. 3. In general terms, the treatment system 800 includes the IMD 60 as described above and a sensor unit that includes two (or more) accelerometers, such as a first accelerometer 804 and a second accelerometer 806. The IMD 60 includes the IPG 64 implanted within the patient 802, and a stimulation lead 808 routed from the IPG 64 to position one or more stimulation elements (e.g., electrode(s)) at a desired stimulation target site. The IPG 64 is programmed (or is prompted) to deliver stimulation signals to the stimulation element via the lead 808 that in turn are applied to the target site. It will be understood that a location of the IPG 64 and of the stimulation lead 808 reflected in FIG. 33 are examples only, and are in no way limiting.

The first and second accelerometers 804, 806 can each be of a type known in the art, and are biocompatible. The first and second accelerometers 804, 806 are, following final implant, maintained in a spaced arrangement. For example, the first accelerometer 804 can be formed with or carried by the IPG 64, and the second accelerometer 806 can be mounted to or carried by the stimulation lead 808. Other techniques for maintaining the accelerometers 804, 806 in a spaced apart arrangement following implant are also acceptable. Regardless, the spaced apart arrangement of the accelerometers 804, 806 allows the sensor unit to gain positional information of an event of interest as well as relative accelerations that may be indicative of a leak causing event or other event. Similarly, angle changes (e.g., akin to a gyroscope) can also be employed. In general terms, signals from the accelerometers 804, 806 can be processed relative to each other to predict or determine that a designated event has occurred or is likely to occur. Alternatively or in addition, comparison or processing of the signals from the accelerometers 804, 806 relative to one another can implicate other patient scenarios of possible interest, for example a posture of the patient. For example, if the signal from one of the accelerometers 804, 806 indicates that it is facing “down” and the other does not (due to gravitational pull), it can be predicted or determined that the patient is bending or has assumed a posture other than upright.

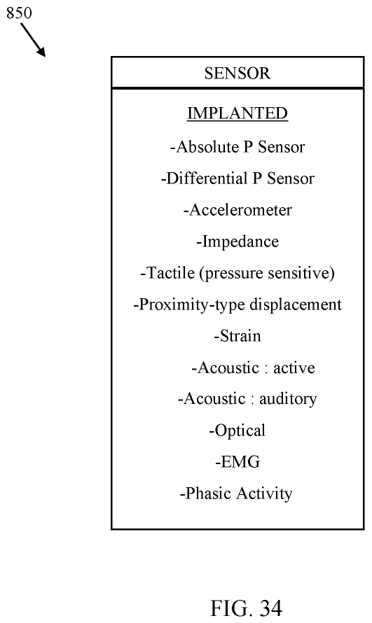

FIG. 34 presents a summary 850 of some of the other sensor formats/types in addition to those of FIG. 18 that may be employed with the treatment systems and methods of the present disclosure, including implanted sensors such as a tactile (pressure sensitive) sensor, strain measuring sensor, and proximity-type positional sensor as described above, as well as an acoustic sensor, an optical sensor, an EMG sensor and a phasic activity sensor. The optional active-type acoustic sensor can be of a type known in the art, and placed to sense volume or a designated event(s), for example an ultrasound sensor. In some embodiments, the active-type acoustic sensor can be operated on a periodic basis to conserve energy. Similarly, an auditory-type acoustic sensor (e.g., a microphone) can be implanted in the body (intra-abdominally, in the perineum, or in a subcutaneous space) at a location likely to detect sound waves generated during coughing or similar events. The optional optical sensor can be of a type known in the art and can located to detect information indicative of position and/or pressure via changes in the patient's diaphragm. For example, optical sensors (e.g., using light fibers instead of traditional electrical conductors) can be capable of measuring tissue pressure, IAP, and/or displacement of tissue. The optional EMG sensor can be of a type known in the art, configured and positioned to detect EMG activity of the pelvic floor, abdominal wall, paraspinous (or paraspinal) muscles, etc. The optional phasic activity sensor includes an electrode or the like configured and located to sense phasic activity (or changes in tonic activity) of the pudendal nerve or branches of the pudendal nerve, affording the ability to monitor these changes and then used to trigger stimulation back to the pudendal nerve through the same or separate electrode. As a point of reference, the phasic activity of the pudendal nerve changes prior to a cough, sneeze, Valsalva, or similar potential leak causing event.

One or more or all of the sensing modalities of at least FIGS. 31-34 can entail one or more sensors implanted in a subcutaneous space of the patient. In other embodiments, the sensor(s) can be secured in an intramuscular location, intra-abdominal location (e.g., in the pelvic floor), out of the intra-abdominal cavity (e.g., the perineal space), etc.

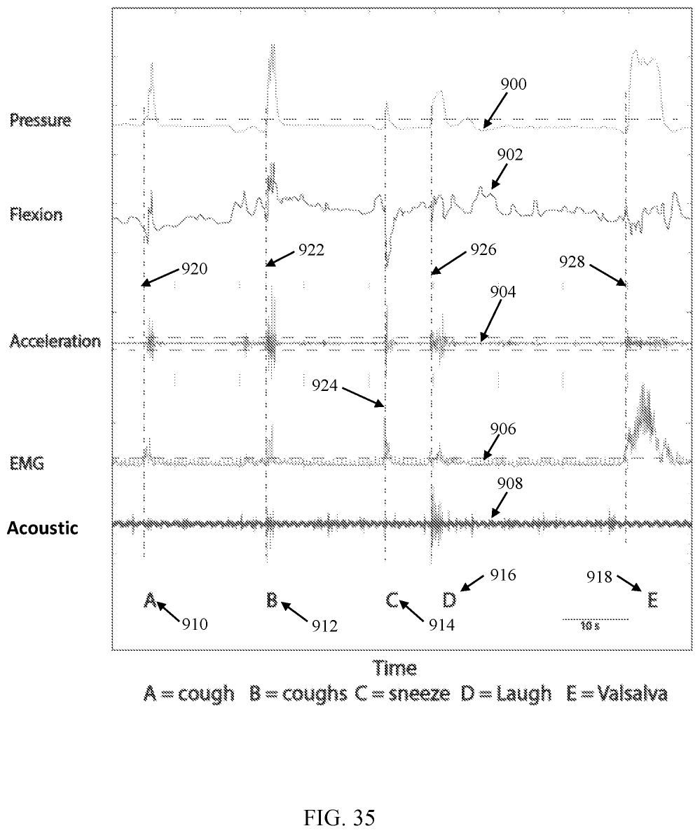

One or more of the sensing modalities described above can be useful as a surrogate for direct IAP (or other SUI causing event) sensing at least with respect so some potential events of interest. Sensing modality viability is further evidenced by FIG. 35 in which signals from several hypothetical sensors in response to several potential leak events experienced by a patient are shown. The sensor formats reported in FIG. 35 include an IAP signal trace 900 from a sensor configured and located to directly detect IAP of the patient, and in particular a fluid filled balloon catheter attached to an external laboratory pressure transducer (it being understood that is but one IAP sensor format). Also shown is a flexion signal trace 902 from a sensor configured and located to measure or detect a change in angle between flank and anterior abdominal surfaces of the patient. An acceleration signal trace 904 represents the magnitude of three dimensional acceleration recorded by an accelerometer-type sensor located on the patient's back near the lumbar spine. As a point of reference, the acceleration signal trace 904 is illustrated as a raw magnitude; in other embodiments as described below, and the raw magnitude signal can be processed for other event detection capabilities. Regardless, the magnitude was computed as the square root of the sum of the squares of each of the x, y, and z acceleration components (i.e., magnitude=(x²+y²+z²)^{1/2}). An EMG signal trace 906 is reported from a sensor configured and located to sense signals from the paraspinal muscles of the patient. As a point of reference, the EMG signal trace 906 of FIG. 35 is a processed signal derived from electrodes placed parallel to the lumbar spine to record signals from the paraspinal muscles. An acoustic signal trace 908 is representative of a raw signal from a microphone sensor placed inside the abdomen.

The data or signal traces 900-908 of FIG. 35 cover the same time period, and during which the following events occurred in the patient: a single cough 910, multiple coughs 912, a sneeze 914, a laugh 916, and Valsalva (bearing down with abdominal muscles while holding breath) 918. Horizontal dashed lines are provided with the IAP signal trace 900, the acceleration signal trace 904, and the EMG signal trace 906 in FIG. 35 to ease the visualization of potential detection thresholds.

A review of FIG. 35 confirms that the IAP signal trace 900, and thus the IAP sensor, is highly efficacious in predicting each of the events 910-918; as such, a comparison of the IAP signal trace 900 with the remaining signal traces 902-908 illustrates the relative strengths of other sensor formats. To assist in this review, and as an aid in visualizing the synchronization of the signal traces 900-908, the approximate timing of the on-set of each of the events 910-918 is noted by a dashed line in FIG. 35 (i.e., dashed line 920 indicates approximate on-set of the single cough; dashed line 922 indicates approximate on-set of multiple coughs; dashed line 924 indicates approximate on-set of the sneeze; dashed line 926 indicates approximate on-set of the laugh; dashed line 928 indicates approximate on-set of Valsalva).

The flexion signal trace 902 was fairly responsive to each of the events 910-918, more prominently for the cough 910, multiple coughs 912, and the sneeze 914. Thus, a sensor or sensor unit formatted and arranged to detect a change in angle between the flank and anterior abdominal muscles of a patient is a highly viable sensing modality (e.g., as a surrogate for, or alterative to, a direct IAP sensor) to predict or detect these and similar events. The acceleration signal trace 904 was fairly responsive to each of the events 910-918, more prominently for the cough 910, multiple coughs 912, the sneeze 914, and the laugh 916. Thus, a sensor or sensor unit formatted and arranged to detect acceleration near a lumbar spine of a patient is a highly viable sensing modality (e.g., as a surrogate for, or alterative to, a direct IAP sensor) to predict or detect these and similar events. The EMG signal trace 902 was responsive to each of the events 910-918, more prominently for Valsalva 918. Thus, a sensor or sensor unit formatted and arranged to detect EMG from paraspinal muscles is a highly viable sensing modality (e.g., as a surrogate for, or alterative to, a direct IAP sensor) to predict or detect these and similar events. The acoustic signal trace 908 was fairly responsive to each of the events 910-918, more prominently for the laugh 916. Thus, a sensor or sensor unit formatted and arranged to detect sound waves of a patient (e.g., a microphone within the patient's body such as provided with the IPG or separately in the subcutaneous, intra-abdominal, perineal, pelvic floor, etc.) is a highly viable sensing modality (e.g., as a surrogate for, or alterative to, a direct IAP sensor) to predict or detect these and similar events.

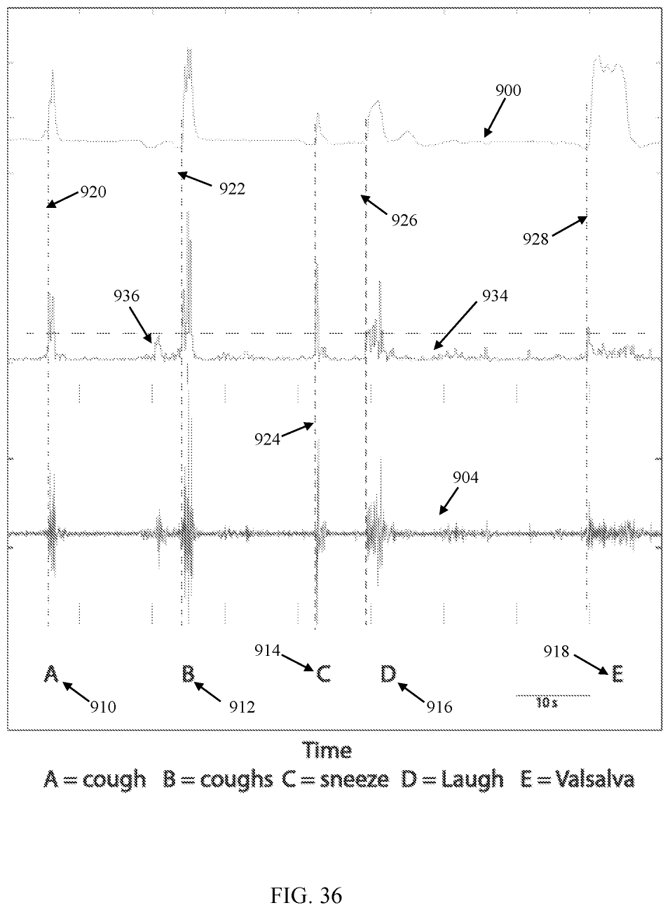

The acceleration signal trace 904 reflected by FIG. 35 is but one example of how an acceleration signal could be considered or reviewed for purposes of predicting or designating the occurrence of one or more events, such as the events 910-918. For example FIG. 36 illustrates the IAP signal trace 900 and the acceleration signal trace 904 reported in FIG. 35 (in conjunction with the events 910-918 and on-set timings 920-928 as described above) along with a processed version of the acceleration signal trace 904 depicted as an acceleration envelope trace 934. The acceleration envelope trace 934 is generated by envelope detection of the signal magnitude of the acceleration signal trace 904. The acceleration envelope trace 934 can serve to more distinctly identify occurrence of each of the events 910-918. Moreover, and as identified generally at 936 in FIG. 36, it is seen that the acceleration envelope trace 934 exhibits a distinct variation just prior to the on-set of the multiple cough event 912. Thus, a sensor or sensor unit formatted and arranged to detect acceleration near a lumbar spine of a patient is a highly viable sensing modality (e.g., as a surrogate for, or alterative to, a direct IAP sensor) for predicting events such as multiple coughs, allowing the treatment systems of the present disclosure to deliver treatment immediately in advance of a potential leak causing event, rather than reacting to a potential leak causing event after the event has started.

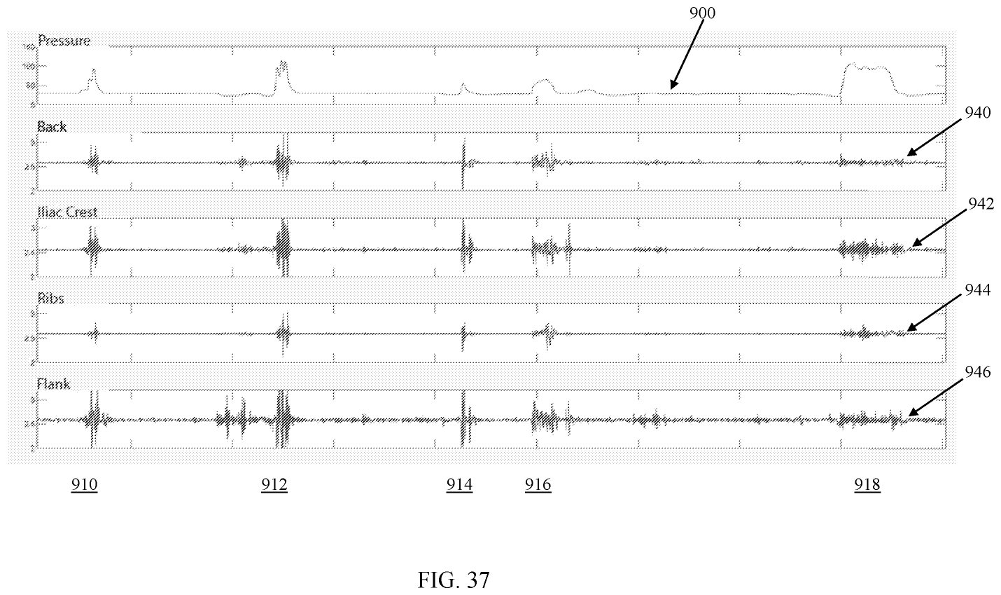

The lumbar spine is but one location from which acceleration information useful for detecting or predicting one or more events, such as one or more of the potential leak causing events 910-918, can be made. A sensors or sensor unit formatted and arranged to detect acceleration at or near other anatomical locations can be utilized in other embodiments of the present disclosure. For example, FIG. 37 reports the signals generated by an accelerometer located near the patient's spine (or “Back”), an accelerometer located next to the patient's iliac crest, an accelerometer located next to the patient's ribs, and an accelerometer located in the lateral abdomen (or “Flank”) over the same time period as FIG. 35 (and during which the patient experienced the single cough event 910, the multiple coughs event 912, the sneeze event 914, the laugh event 916, and the Valsalva event 918) as raw magnitude signal traces 940, 942, 944, and 946. As a point of reference, the Back signal trace 940 is the same as the acceleration signal trace 904 of FIG. 35. Further, the IAP signal trace 900 as described above is displayed in FIG. 37 for comparison. As revealed by FIG. 37, each of the accelerometer locations generated information useful as a viable sensing modality (e.g., as a surrogate for, or alterative to, a direct IAP sensor) in detecting or predicting occurrence of at least the reported events 910-918.

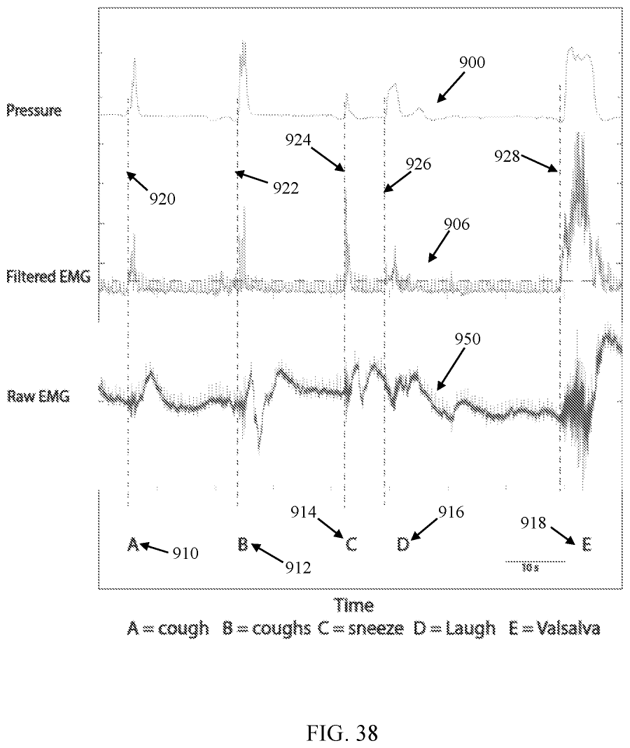

The EMG signal trace 906 shown in FIG. 35 can be derived or generated in various manners. In some non-limiting examples, and with reference to FIG. 38, a raw EMG signal 950 from paraspinal electrodes can be filtered with a high pass filter (e.g., with a corner frequency of 20 Hz) to remove the movement artifact prior to demeaning, rectifying and smoothing of the data, and resulting in the EMG signal trace 906. As a point of reference, the IAP signal trace 900, the events 910-918, and the on-set indicators 920-928 described above are also displayed in FIG. 38. With the example of FIG. 38, electrical signals from the heart are evident, but could be filtered out or ignored by the event detection algorithm utilized by the corresponding treatment system. Once again, a comparison of the on-set indicators 920-928 with EMG signal trace 906 and the raw EMG signal 950 illustrates that each of the potential leak events 910-918 could be detected, for example by employing a simple threshold crossing detection algorithm. It is further noted from FIG. 38 that the paraspinal EMG information can be particularly useful for detecting the onset of a Valsalva maneuver (i.e., the Valsalva event 918), an event that might otherwise be difficult for other sensor formats/locations to detect due to lack of overt movement.

### Other Algorithms

Returning to FIG. 3, in some embodiments the systems and methods of the present disclosure can include or incorporate one or more stimulation methods or algorithms for prompting the IMD 60 to initiate delivery of, cease delivery of, and/or modulation one or more of the stimulation signals (e.g., via programming provided with the control portion 70) in place of, or in addition to, the methods and algorithms described above. In some embodiments, the algorithms can be acted upon or utilize sensed patient-related information. In some examples, the stimulation signal is initiated and/or modulated based on sensed patient information. In some examples, one or more of the amplitude, rate, and pulse duration of the stimulation signal is modulated based upon, for example, the sensed patient information. Alternatively or in addition, the duty cycle of the stimulation signal is altered in response to the sensed patient information.

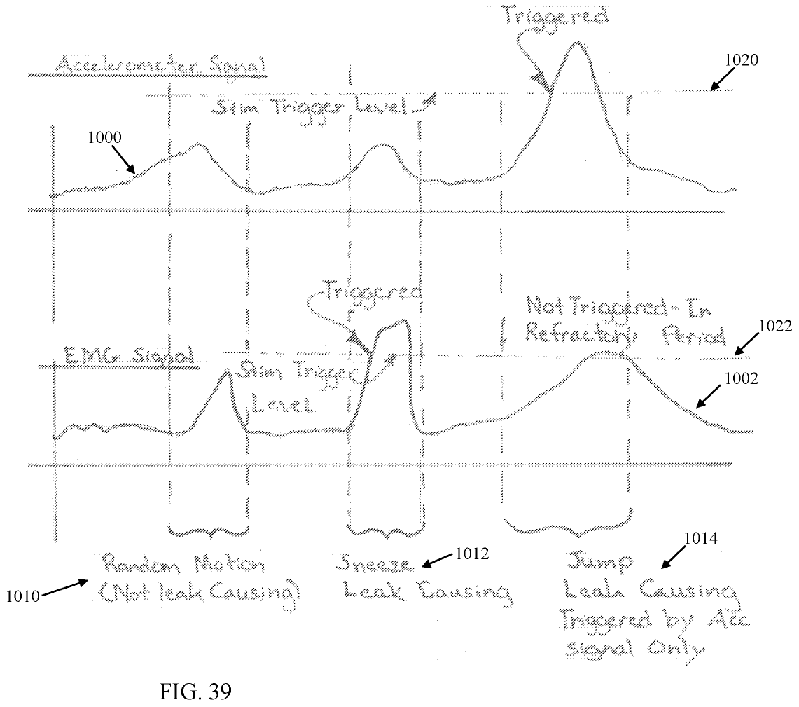

In some embodiments, methods and algorithms of the present disclosure can implement two or more sensing signals to determine one or more stimulation delivery parameter (e.g., timing or triggering of stimulation). For example, where two or more sensors (or sensor units) are provided that sense or detect patient-related information and that differ from one another by one or both of format and location, the so-provided sensor information can be considered and acted upon in a complimentary manner by some algorithms of the present disclosure. With these and related embodiments, some algorithms of the present disclosure can leverage a higher likelihood or ability of certain sensor types and/or locations to detect or predict occurrence of a particular event or scenario as compared to other sensor types and/or locations. FIG. 39 represents one example of a stimulation protocol or algorithm implemented by some embodiments of the present disclosure and responsive to a combination of sensing signals. In particular, FIG. 39 presents a simplified representation of a signal (“Accelerometer Signal”) 1000 generated by an accelerometer located near the patient's spine and a simplified representation of a signal (“EMG Signal”) 1002 generated by an EMG sensing unit located near the patient's paraspinal muscles or erector spinae. In FIG. 39, the signals 1000, 1002 are generated over the same period of time and during which the patient experiences or engages in various events, such as random motion 1010, a sneeze 1012, and jumping 1014. With some algorithms of the present disclosure, a “stim trigger level” 1020 is established with respect to the Accelerometer Signal 1000; when the Accelerometer Signal 1000 exceeds the stim trigger level 1020, the IPG 64 (FIG. 3) is prompted to deliver stimulation (e.g., at a predetermined level, tuned stimulation, etc.) to the patient for one or both of a pre-determined period of time or until the Accelerometer Signal 1000 drops below the stim trigger level 1020. Similarly, a “stim trigger level” 1022 is established with respect to the EMG Signal 1002; when the EMG Signal 1002 exceeds the stim trigger level 1022, the IPG 64 is prompted to deliver stimulation (e.g., at a predetermined level, tuned stimulation, etc.) to the patient for one or both of a pre-determined period of time or until the EMG Signal 1002 drops below the stim trigger level 1022.

With the above in mind, during the time period in which the patient is engaged in random motion 1010, the Accelerometer Signal 1000 remains below the stim trigger level 1020 and the EMG Signal 1002 remains below the stim trigger level 1022. Under these and other circumstances, some algorithms of the present disclosure can be configured such that no stimulation is delivered to the patient and/or that minimal or base level of stimulation is delivered (e.g., some algorithms of the present disclosure can actively or inferentially designate that with the random motion event 1010 is unlikely to cause a leak and thus that no or minimal stimulation is needed).

During the time period which the patient experiences a sneeze 1012, the Accelerometer Signal 1000 remains below the stim trigger level 1020. However, the EMG Signal 1002 exceeds the stim trigger level 1022. Under these and similar circumstances, some algorithms of the present disclosure can be configured to prompt the delivery of stimulation (or increase a level of currently-delivered stimulation) to the patient, notwithstanding a patient status implicated by the Accelerometer Signal 1000. Thus, a potential for leaking otherwise raised by the sneeze event 1012 is addressed.

During the time period which the patient jumps 1014, the Accelerometer Signal 1000 exceeds the stim trigger level 1020. The EMG Signal 1002 also eventually exceeds the stim trigger level 1022, but does so later in time as compared to the Accelerometer Signal 100. Under these and similar circumstances, some algorithms of the present disclosure can be configured to prompt the delivery of stimulation (or increase a level of currently-delivered stimulation) to the patient immediately upon the Accelerometer Signal 1000 exceeding the stim trigger level 1020, notwithstanding a patient status implicated by the EMG Signal 1002. Thus, a potential for leaking otherwise raised by the jump event 1014 is addressed. In some optional embodiments, the algorithms of the present disclosure can further be configured or programmed to end the delivery of stimulation (or decrease a level of stimulation being delivered) once the Accelerometer Signal 1000 drops below the stim trigger level 1020 notwithstanding the fact that at that same point in time, the EMG Signal 1002 remains above the stim trigger level 1022 (i.e., a “refraction period”). In other embodiments, stimulation can be delivered (or not decreased) until the EMG Signal 1002 drops below the stim trigger level 1022.

The non-limiting example of FIG. 39 leverages a combination of EMG information from the paraspinal muscles and accelerometry in an algorithm for detecting changes that could result in a leak event. These and related algorithms can be useful because, for example, gravitational forces that cause IAP increases (e.g., a jump, sudden bump while riding in the car, etc.) may be detectable using an accelerometer but might not cause increased EMG activity of the paraspinal muscles. While FIG. 39 illustrates the two sensor signals considered by the algorithm as the Accelerometer Signal 1000 and the EMG Signal 1002, multiple other sensor formats/locations are also acceptable that may or may not include an accelerometer and/or paraspinal muscle EMG. For example, EMG activity of the pelvic floor and abdominal wall may see sharp increases prior to the increase in IAP in advance of a cough or sneeze and thus can be a more effective trigger signal for these (and other) events, where a complimentary pressure sensor in the abdomen can be a better indicator of a possible external urinary sphincter muscle leak even in a slowly-building pressure event such as Valsalva. These and other algorithms of the present disclosure can consider or act upon sensor signal(s) in a wide variety of fashions in addition to, or as an alternative to, the representations of FIG. 39. For example, one or aspects of sensor signal(s) can be determined and considered such as, but not limited to, higher order derivatives of the signal, running average, weighted average, combination of characteristics, etc. Optionally, an “and” as well as an “or” function can be employed. By way of non-limiting example, some useful algorithms can include triggering stimulation when a particular sensor signal exceeds a specific level of the first order signal (e.g., pressure) combined with a second order signal level (e.g., derivative/rate of change in pressure) or a rate of change of the mean of an EMG signal.

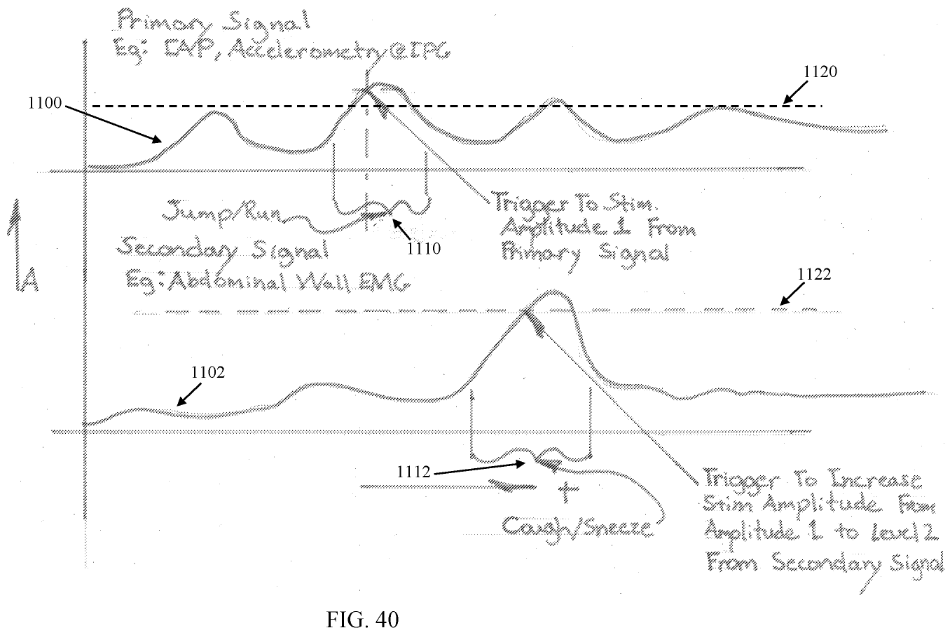

FIG. 40 represents another example of a stimulation protocol or algorithm implemented by some embodiments of the present disclosure and responsive to a combination of sensing signals. In particular, FIG. 40 presents a simplified representation of a signal (“Primary Signal”) 1100 generated by a sensor or sensor unit formatted and located to sense a parameter treated as highly indicative of an event or scenario for which stimulation will be beneficial (e.g., an IAP sensor, an accelerometer, etc.), and a simplified representation of a signal (“Secondary Signal”) 1102 generated by a sensor or sensor unit apart from the sensor generating the Primary Signal 1100 (e.g., the Secondary Signal 1102 can be generated by an EMG sensor located along the abdominal wall of the patient). In FIG. 40, the signals 1100, 1102 are generated over the same period of time and during which the patient experiences or engages in a jump event 1110 and a cough or sneeze event 1112. With some algorithms of the present disclosure, a “stim trigger level” 1120 is established with respect to the Primary Signal 1100; when the Primary Signal 1100 exceeds the stim trigger level 1120, the IPG 64 (FIG. 3) is prompted to deliver stimulation (e.g., at a predetermined level, tuned stimulation, etc.) to the patient for one or both of a pre-determined period of time or until the Primary Signal 1100 drops below the stim trigger level 1120. A “stim increase level” 1122 is established with respect to the Secondary Signal 1102; when the Secondary Signal 1102 exceeds the stim increase level 1122, the IPG 64 is prompted to increase a level (e.g., amplitude) of stimulation being delivered to the patient.

During the time period which the patient jumps 1110, the Primary Signal 1100 exceeds the stim trigger level 1120 while the Secondary Signal 1102 does not exceed the stim increase level 1122. Under these and similar circumstances, some algorithms of the present disclosure can be configured to prompt the delivery of stimulation at a first level (e.g., “Amplitude 1”). Thus, a potential for leaking otherwise raised by the jump event 1110 is addressed.

During the time period which the patient experiences the cough or sneeze 1112, the Primary Signal 1100 exceeds the stim trigger level 1120 and the Secondary Signal 1102 exceeds the stim increase level 1122. Under these and similar circumstances, some algorithms of the present disclosure can be configured to prompt the delivery of stimulation at a second level (e.g., “Amplitude 2”) or increase delivered stimulation from Amplitude 1 to Amplitude 2. Thus, a potential for leaking otherwise raised by the cough or sneeze event 1112 is addressed. Moreover, by combining information from two (or more) sensor sources, as evidence of a leak event increases, the stimulation delivered to the patient also increases. Other, similar algorithms or protocols can be utilized. For example, some stimulation algorithms of the present disclosure can be formatted such that when the Primary Signal 1100 reaches the stim trigger level 1120, a first level of stimulation is delivered unless the Secondary Signal 1102 exceeds the stim increase level 1120 in which case a second level of stimulation is delivered and the Primary Signal 1100 is ignored. Alternatively or in addition, if the Primary Signal 1100 reaches the stim trigger level 1120 and the Secondary Signal 1102 reaches the stim increase level 1120 at the same time (or within a set interval), then a third level of stimulation is delivered.

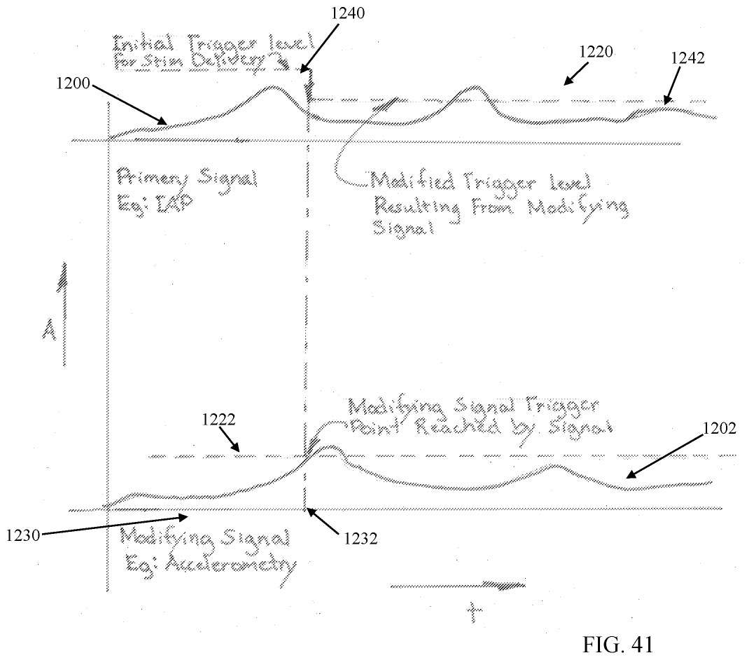

FIG. 41 represents another example of a stimulation protocol or algorithm implemented by some embodiments of the present disclosure and responsive to a combination of sensing signals. In particular, FIG. 41 presents a simplified representation of a signal (“Primary Signal”) 1200 generated by a sensor or sensor unit formatted and located to sense a parameter treated as highly indicative of an event or scenario for which stimulation will be beneficial (e.g., an IAP sensor, an accelerometer, etc.), and a simplified representation of a signal (“Secondary Signal”) 1202 generated by a sensor or sensor unit apart from the sensor generating the Primary Signal 1200 (e.g., the Secondary Signal 1202 can be generated by an accelerometer). In FIG. 41, the signals 1200, 1202 are generated over the same period of time. With some algorithms of the present disclosure, a “stim trigger level” 1220 is established with respect to the Primary Signal 1200; when the Primary Signal 1200 exceeds the stim trigger level 1220, the IPG 64 (FIG. 3) is prompted to deliver stimulation (e.g., at a predetermined level, tuned stimulation, etc.) to the patient for one or both of a pre-determined period of time or until the Primary Signal 1200 drops below the stim trigger level 1220.

With some algorithms of the present disclosure, a “sensitivity level” 1222 is established with respect to the Secondary Signal 1202. When the Secondary Signal 1202 exceeds the sensitivity level 1222, the algorithm modifies the stim trigger level 1220. For example, during a first time period 1230, the Secondary Signal 1202 is below the sensitivity level 1222; under these circumstances, the stim trigger level 1220 is remains an Initial Trigger Level 1240. At a later point in time 1232, the Secondary Signal 1202 exceeds the sensitivity level 1222; under these circumstances, the stim trigger level 1220 is lowered to a Modified Trigger Level 1242. The stim trigger level 1220 can remain at the Modified Trigger Level 1242 for a predetermined length of time, can return to the Initial Trigger Level 1240 upon detection of a certain event, etc. Regardless, by modifying the stim trigger level 1220 in response to information from the Secondary Signal 1202, overall sensitivity of the treatment system can be heightened or modified. Thus, in some examples, these and other optional algorithms of the present disclosure can account for the patient experiencing or engaging in actions or activities that are deemed to be suppressing sensitivity of the sensor or sensor unit that is generating the Primary Signal 1200 (e.g., a body position of the patient may suppress sensitivity of an IAP sensor).

The algorithms implicated by FIGS. 39-41 can be implemented with variations from the above explanations. For example, any of the algorithms of FIGS. 39-41 can be modified to act upon or utilize sensed information from the patient other than the sensor or sensor formats mentioned. Further, any of the algorithms of FIGS. 39-41 can be modified to act upon or utilized sensed information from three (or more) sensors.

### Other Microstimulators

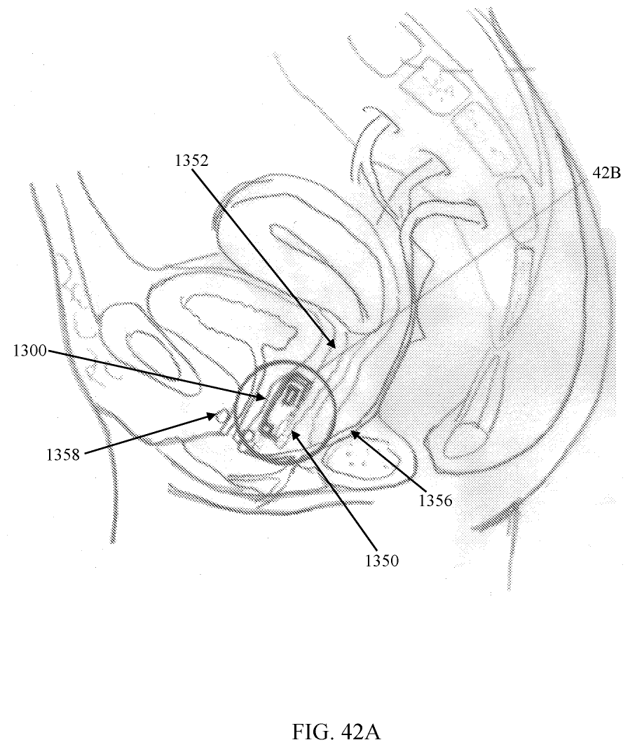

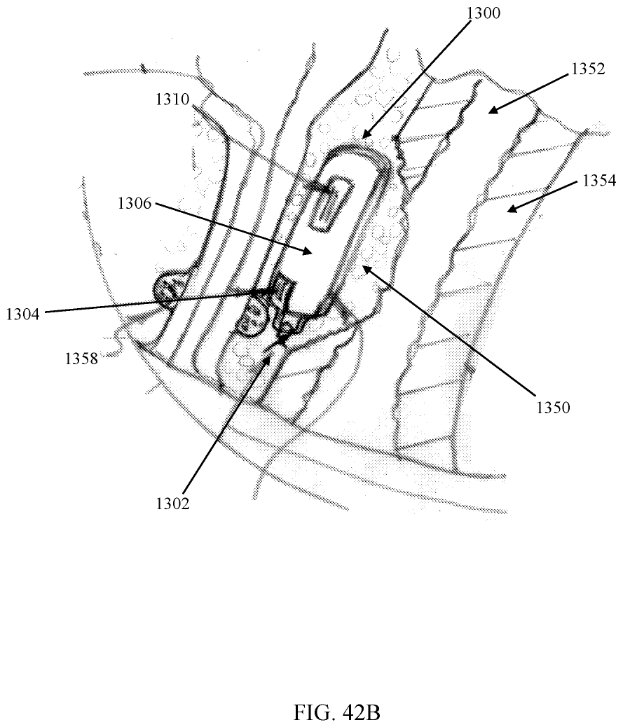

In some embodiments, the systems and methods of the present disclosure can include or incorporate a microstimulator, such as the microstimulator 370 and related, optional components described above with respect to FIGS. 17A and 17B. With this in mind, some systems and methods of the present disclosure can be useful with female patients and can include placement of a microstimulator surgically through the vaginal wall and anchored, for example, in the deep perineal space. For example, FIGS. 42A and 42B illustrate a microstimulator 1300 implanted at a target site 1350 within or adjacent a vagina 1352 in accordance with some systems and methods of the present disclosure. The microstimulator 1300 can be akin to or include one or more of the features associated with the microstimulator 370 described above. Other, optional features of the microstimulator 1300 are provided below.

In some embodiments, the microstimulator 1300 can be delivered to the target site 1350 laparoscopically or on a minimally invasive basis via a transvaginal approach. These and related techniques can be akin to known procedures for placement of a vaginal sling or a transvaginal mesh, such as a single incision sling (or “mini-sling”) procedure, a midurethral sling procedure, a pubovaginal sling procedure, or similar procedures commonly practiced by urologists. In some embodiments, the procedure can be performed under local or regional anesthesia. A small incision is made along a wall 1354 of the vagina 1352 at the target site 1350; additional incisions may also be made. The microstimulator 1300 is then transvaginally delivered to and into the incision at the target site 1350. Other delivery techniques or procedures can also be employed. For example, with some optional methods or procedures, a short stimulation lead or leads (not shown) can be transvaginally placed during transvaginal delivery of the microstimulator 1300. The stimulation lead can be employed to facilitate specific nerve location stimulation (including bilateral nerve stimulation) via testing, and/or external urinary sphincter or other muscle monitoring can be performed via the stimulation lead to confirm a desired stimulation effect.

In some embodiments, the target site 1350 is at or in the deep perineal space of the patient. As a point of reference, the deep perineal space (or deep perineal pouch) is the anatomical space enclosed in part by the perineum, and is located superior to the perineal membrane. The deep perineal space can be considered to be bordered inferiorly by the perineal membrane (also known as the inferior fascia of the urogenital diaphragm), and typically presents as a triangular, trilaminar space with external urethra sphincter, deep transverse perineal, and compressor urethrae (in females). In other embodiments, the microstimulator 1300 can be implanted at other target sites accessible via a transvaginal approach that may or may not include the deep perineal space.

The microstimulator 1300 can be anchored to the vaginal wall 1354 (or other anatomical structure) by one or more sutures 1302. Other anchoring techniques or devices are also acceptable. For example, in some embodiments the microstimulator 1300 can include or carry one or more anchoring devices (e.g., deployable tines or barbs) that secure the microstimulator to or within the vaginal wall 1354 or adjacent the vagina 1352.

Regardless of how the microstimulator 1300 is secured to the vaginal wall 1354 (or other anatomical structure), in some embodiments the transvaginal delivery and implantation techniques of the present disclosure promote ready or straightforward access to the microstimulator 1300 on a minimally invasive basis following implant. The target site/implant location 1350 is readily accessible transvaginally, allowing a clinician to physically interface with the microstimulator 1300 for various reason following implant, for example to remove, replace, or recharge the microstimulator 1300. In some embodiments, the microstimulator 1300 is positioned upon final implant for transvaginal recharge.

With some systems and methods of the present disclosure, a location of the target site 1350 is selected in tandem with a configuration of the microstimulator 1300 such that following implant, stimulation element(s) (e.g., electrodes) 1304 carried on a housing 1306 of the microstimulator 1300 is/are proximate anatomy of interest. For example, in some embodiments, the microstimulator 1300 is positioned such that the stimulation element(s) 1304 is proximate a pudendal nerve 1356 (e.g., a deep perineal branch of the pudendal nerve 1356). With these and related embodiments, the microstimulator 1300 can be operated to apply stimulation energy to the pudendal nerve 1356 in accordance with any of the stimulation methods of the present disclosure. Alternatively or in addition, the microstimulator 1300 can be positioned such that the stimulation element(s) 1304 is proximate an external urinary sphincter muscle 1358. With these and related embodiments, the microstimulator 1300 can be operated to apply stimulation energy to the external urinary sphincter muscle 1358 in accordance with any of the stimulation methods of the present disclosure. Alternatively or in addition, the microstimulator 1300 can be positioned such that the stimulation element(s) 1304 is proximate another anatomical structure of interest to apply stimulation energy thereto, for example direct muscle stimulation of the pelvic floor. In some embodiments, the microstimulator 1300 is configured to actively generate stimulation energy (e.g., via a power source or battery included with the microstimulator 1300). In other embodiments, the microstimulator 1300 has a passive format and utilizes an external device to wirelessly transmit energy to activate stimulation.

With some systems and methods of the present disclosure, a final implant location of the microstimulator 1300 can be proximate or at a mid-line of the patient. With these and related embodiments, the microstimulator 1300 can include two (or more) stimulation elements 1304 and the microstimulator 1300 is operable to provide bilateral stimulation to the patient (e.g., a first one (or more) of the stimulation elements 1304 is positioned proximate a left pudendal nerve branch, and a second one (or more) of the stimulation elements is positioned proximate a right pudendal nerve branch, the left and right pudendal nerve branches can each be stimulated (simultaneously, discretely, etc.) during operation of the microstimulator 1300.

In some embodiments, the microstimulator can include or carry one or more sensors 1310 on the housing 1306. The sensor(s) 1310 can assume various forms, for example formatted to sense or detect a patient parameter(s) of interest at or proximate the target site 1350. A signal from the sensor(s) 1310 can be can be processed or acted upon by one or more algorithms described in the present disclosure. For example, the sensor 1310 can be formatted to detect at least one of IAP, tissue pressure, sound waves (e.g., an acoustic sensor capable of detecting a cough or sneeze), EMG, acceleration effects, electrical nerve activity (ENG) or other parameters of potential interest, for example a parameter indicative of an SUI causing event. In some embodiments, the sensor 1310 carried by the housing 1306 can be a pressure sensor, with the final implant location of the microstimulator 1300 naturally locating the pressure sensor 1310 to directly sense pressure of the pelvic floor.

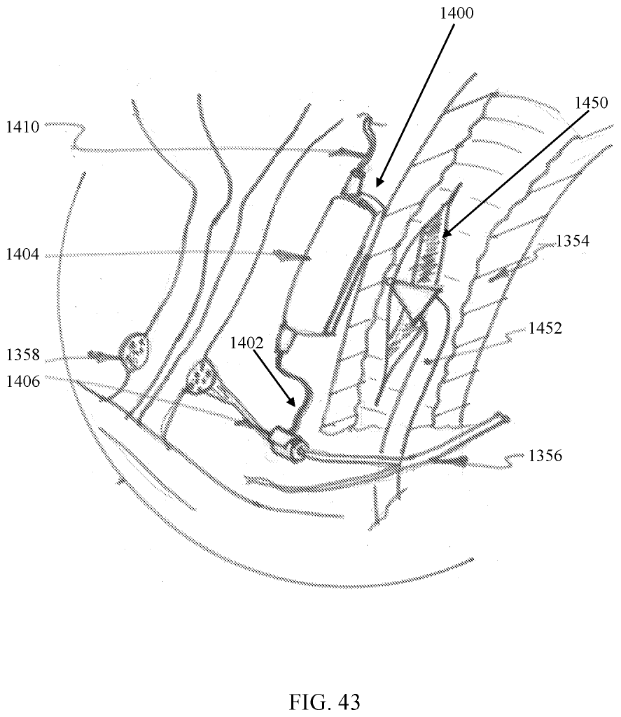

In addition to, or as an alternative to, the stimulation element(s) 1304 carried on the housing 1306 of the microstimulator 1300, in other embodiments a short lead or the like can be included. For example, FIG. 43 illustrates another implanted microstimulator 1400 in accordance with systems and methods of the present disclosure. The microstimulator 1400 can have any of the forms described elsewhere (e.g., any of the features described with respect to the microstimulator 370 of FIGS. 17A and 17B or the microstimulator 1300 of FIGS. 42A and 42B). As with the embodiments of FIGS. 42A and 42B, the microstimulator 1400 can be transvaginally delivered and placed at a target site via an incision 1450 formed in the vaginal wall 1354 by a surgical access device 1452 (illustrated generally). The so-delivered microstimulator 1400 can be secured to the vaginal wall 1354 as described above with respect to FIGS. 42A and 42B (e.g., the microstimulator 1400 can be transvaginally implanted on a minimally invasive basis). With the non-limiting example of FIG. 43, a stimulation lead-type body 1402 extends from a housing 1404 of the microstimulator 1400 and carries one or more stimulation elements 1406 (referenced generally) in a format appropriate for securement at or proximate an anatomical structure of interest. For example, the stimulation element(s) 1406 can be provided with or carried by a stimulation cuff body that is secured over a desired location (e.g., branch) of the pudendal nerve 1356. Alternatively, electrode lead configurations other than a cuff are equally acceptable (e.g., cylindrical lead, “paddle” shaped lead carrying an array of electrodes to optimize capture of specific branches (and fascicles) of the pudendal nerve or other nerve, etc.). For example, the stimulation element(s) 1406 can be provided with a lead body configured for securement at or proximate the external urinary sphincter muscle 1358. Alternatively or in addition, with some systems and methods of the present disclosure, a stimulation element-carrying lead extends from the microstimulator housing 1404 and is sized and shaped for securement to (and thus delivery of stimulation energy to) other anatomical structure(s) of interest; with these and related embodiments, the implantation procedures can include inserting or tunneling the stimulation lead 1402 through tissue. Regardless, the stimulation lead 1402 electrically connects the stimulation element(s) 1406 to circuitry within the housing 1404, such that the microstimulator 1400 is operable to deliver stimulation energy to the stimulation element(s) 1406, for example utilizing one or more of the stimulation algorithms of the present disclosure.

In some embodiments, two (or more) of the stimulation leads 1402 can be provided. With these and related embodiments, a final implant location of the microstimulator 1400 can be proximate or at a mid-line of the patient. From this location a first stimulation lead 1402 can be located to place the corresponding stimulation element(s) 1406 on a left pudendal nerve branch, and a second stimulation lead 1402 can be located to place the corresponding stimulation element(s) 1406 on a right pudendal nerve branch. As a point of reference, the deep perineal branches of the left and right pudendal nerves converge toward the mid-line as they innervate the external urinary sphincter 1358, and thus can be readily accessed by leads extending from the microstimulator housing 1404. In some embodiments, then, the microstimulator 1400 can be operated to provide bilateral stimulation, thereby improving the amount of contraction of the external urinary sphincter 1358 (or other muscle(s) innervated by the pudendal nerve or in the pelvic floor but not innervated by the pudendal nerve such as the levator ani).

FIG. 43 further reflects that in some optional embodiments, at least one sensing lead 1410 extends from the microstimulator housing 1404. The sensing lead 1410 can generally be of a type known in the art, and includes or carries one or more sensing elements (not shown), such as an electrode or the like. With some systems and methods of the present disclosure, the sensing lead 1410 is arranged and secured so as to locate the sensing element(s) proximate or on a muscle of the pelvic floor, such as a pelvic floor muscle that reacts to an increase in IAP. In some embodiments, EMG or similar signals generated by the muscle(s) to which the sensing lead 1410/sensing element is secured can be used, for example, to trigger stimulation when the signal implicates an increase in IAP or of a potential leak causing event via algorithms of the present disclosure. In this regard, muscles of the pelvic floor and adjacent areas that can be monitored via the sensing lead 1410 with some systems and methods of the present disclosure and based upon which initiation, modulation, cessation, etc., of stimulation energy by the microstimulator 1400 is can be controlled include: rectal coccygeal muscle; levator ani, iliococcygeus; levator ani, pubococcygeus; levator ani; puborectalis; levator ani; sphincter ani externus; coccygeus; superficial transverse perineal muscle; deep transverse perineal muscle; external sphincter muscle of female urethra; compressor urethrae; sphincter urethrae; bulbospongiosus; rectus abdominis; internal abdominal oblique; external abdominal oblique; psoas major; iliac muscle; quadratus lumborum; diaphragm; quadratus femoris; obturator externus; obturator internus; gemellus inferior; gemellus superior; piriform; ischiococcygeus; transverse perinei profundus; superficial transverse perineal muscle; ischiocavernosus.

Alternatively or in addition, the sensing lead 1410 can be located to place the corresponding sensor to sense or record an ENG of a nerve of interest. As a point of reference, the phasic (or change in tonic) activity of certain nerves are a precursor to a stress-causing event such as increases in IAP. These nerves include, but are not limited to, the pudendal nerve and its branches, the hypogastric nerve, the pelvic splanchnic nerve, the cavernous nerve, the obturator internus nerve, etc.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. For example, the systems and methods of the present disclosure can be utilized to treat other maladies, such as interstitial cystitis (IC). With IC treatment applications, one or more of the target sites discussed above are implicated. The delivered stimulation energy for treatment of IC can differ from that applied for the treatment of incontinence for example; in some embodiments, a larger and/or higher frequency stimulation energy as compared to that appropriate for treatment of incontinence.

1. A method of treating a bladder and/or bowel dysfunction of a patient, the method comprising:
sensing at least one parameter of the patient indicative of a potential bladder or bowel dysfunction event; and
applying stimulation energy to an anatomical structure of the patient as a function of the sensed parameter to address the potential bladder or bowel dysfunction event.

2-11. (canceled)
